(19) United States
(12) Patent Application Publication
(10) Pub. No.: US 2018/0148783 A1
(43) Pub. Date: May 31, 2018

(54) METHOD OF EPIGENETIC ANALYSIS FOR DETERMINING CLINICAL GENETIC RISK

(71) Applicant: The Johns Hopkins University, Baltimore, MD (US)

(72) Inventors: Andrew P. Feinberg, Lutherville, MD (US); Andrew Ellis Jaffe, Baltimore, MD (US); Juleen Rae Zierath, Lidingoe (SE); Erik Bertil Naeslund, Taeby (SE); Guang William Wong, Lutherville, MD (US)

(21) Appl. No.: 15/541,455

(22) PCT Filed: Jan. 5, 2016
(86) PCT No.: PCT/US16/12217

§ 371 Date: Jul. 3, 2017

Related U.S. Application Data
(60) Provisional application No. 62/100,039, filed on Jan. 5, 2015.

(51) Int. Cl. C12Q 1/6883; A61K 31/7105; A61P 3/00; A61P 3/04; A61P 3/10

(52) U.S. Cl. CPC C12Q 1/6883; A61K 31/7105; A61P 3/00; C12Q 2600/118; A61P 3/10; C12Q 2600/154; A61P 3/04

(57) ABSTRACT

The present invention provides a method for identifying a subject having or at risk of having a metabolic disease, such as diabetes or obesity. The invention is based on an approach to identify candidate genes involved in metabolic diseases, such as obesity and type 2 diabetes (T2D) through epigenetic mechanisms. The method includes identifying in the subject genetic markers correlating differentially methylated regions (DMRs) in the genome with genetic risk loci for the subject and comparing methylation patterns of the markers with a control sample from a subject not having the disease. In another embodiment, the invention also provides a method of treating a subject having or at risk of having a metabolic disease. In another embodiment, the invention provides a method of providing a prognostic evaluation of a subject having or at risk of having a metabolic disease.

### RELATED APPLICATION DATA

This application claims the benefit of priority under 35 U.S.C. §119(e) of U.S. Provisional Patent Application Ser. No. 62/100,039, filed Jan. 5, 2015, the entire contents of which is incorporated herein by reference in its entirety.

### STATEMENT OF GOVERNMENT SUPPORT

This invention was made in part with government support under Grant Nos. DP1 ES022579 and DK084171 awarded by the National Institutes of Health. The United States government has certain rights in this invention.

### INCORPORATION OF SEQUENCE LISTING

The material in the accompanying sequence listing is hereby incorporated by reference into this application. The accompanying sequence listing text file, name JHU3760_1WO_Sequence_Listing, was created on 4 Jan. 2016, and is 30 kb. The file can be assessed using Microsoft Word on a computer that uses Windows OS.

### BACKGROUND OF THE INVENTION

### Field of the Invention

The present invention relates generally to differentially methylated regions (DMRs) in the genome, and more specifically to methods for correlating DMRs with metabolic diseases or disorders.

### Background Information

The basis of modern disease association studies can be predicated on the “common disease common variant hypothesis,” which argues that frequent variants in the general population, that arose at a point of historical population restriction, are associated with genetic variants for common disease. The concept is rooted in the neo-Darwinian synthesis of the previous century, and the population genetic analysis of R. A. Fisher, who argued that complex (multigenic) phenotypes arise additively from individual quantitative trait loci (QTLs). A great deal of effort has been expended on finding associations of common disease with single nucleotide polymorphisms (SNPs). While there have been important successes, the overwhelming majority of genome-wide associations studies (GWAS) have shown associations characterized by low odds ratios, around 70% report odd-ratio below 2, with generally relatively weak genome-wide statistical significance. This is a well-recognized problem in the GWAS community, and has led to discussions of sources of the missing “dark matter” of heritability, reviewed recently in the literature. Alternatives include copy number variants, and rare variants, although copy numbers also appear to account for a relatively small attributable risk of disease, e.g. <1% in schizophrenia. A major goal of funding agencies is to extend sequencing efforts to much larger cohorts, and the identification of the major cause of disease-related genetic variation is essential to fulfill ambitions for personalized medicine, i.e., targeting therapy and disease risk mitigation based on one's genome.

A role for epigenetics in common disease has long been suspected, and a strong relationship with cancer has been shown. It is likely that common disease involves both genetic and epigenetic factors and that epigenetic modification could mark both environmental effects as well as mediate genetic effects. In addition to particular exposure-epigenetic relationships, epigenetic changes with aging support the notion that there is an environmental component to epigenetic variation. Studies of identical twins show greater differences in global DNA methylation in older than in younger twins, consistent with an age-dependent progression of epigenetic change. Global methylation changes over an 11 year span in participants of an Icelandic cohort, and age- and tissue-related alterations in some CpG islands from an array of 1,413 arbitrarily chosen CpG sites near gene promoters, further corroborate the evidence for dynamic methylation patterns over time. Other work, however, has suggested that epigenetic marks, or their maintenance, are themselves controlled by genes, and are thus heritable in the traditional sense and associated with particular DNA variants. This would predict that methylation marks are stable, rather than varying as controlled by changing environments.

A tenet of Origin of Species argues that phenotype is the result of many discrete traits that are individually and exquisitely selected, to quote Darwin, “detecting the smallest grain in the balance of fitness,” which has been described as Newtonian in its dependence on static forces acting in consistent ways. This concept is the basis for quantitative trait loci that has been proposed in the scientific field. This concept has led to the modern basis of population genetics that continuous variation exists within a population, yet selection is on individuals, which has led to models of balancing or purifying selection at the extremes of phenotype. The classic model also has significant limitations in explaining common human disease; common variants can explain only a small fraction of a given disease phenotype, even the most well understood, such as adult-onset diabetes and height.

Epigenetics, the study of non-sequence-based changes in DNA and associated proteins, was first suggested to play a role in evolution through Lamarckian inheritance, that is, direct modification of the genome by the environment, which is then transmitted transgenerationally. Two examples are commonly cited: changes in coat color caused by dietary modifications of DNA methylation of the agouti gene in mice and methylation of the axin-fused allele in kinked tail mice. Both of these examples involve methylation of a retrotransposon LTR sequence, and thus fit into various genetic exceptions to classical Darwinian thinking, including anticipation due to trinucleotide repeat expansion and lateral gene transfer in the evolution of influenza strains. But they have not been shown to be general mechanisms for either speciation or developmental differences across species, so-called “evo-devo,” or for canalization, a term coined to refer to a mechanism by which environmental perturbations during development are corrected by the genetic program, leading to a consistent developmental plan.

Indeed, canalization remains a “black box,” as noted by some in the scientific field. Others have discussed the potential role for Lamarckian inheritance in disease; for example, some have proposed a model of transgenerational epigenetic Lamarckian inheritance and noted that such modifications must persist for many generations to contribute substantially to average risk, which has implications for public health management. Although not disputing an important contribution of Lamarckian inheritance, here the invention provides an alternative view in which genetic modification could provide stochastic phenotypic variation favored by selection in changing environments, and also provide an alternative non-Lamarckian role for epigenetics in evolution.

Thus, there is a need for a genome-scale analysis of DNA methylation to correlate epigenomics and clinical genetic risk.

### SUMMARY OF THE INVENTION

The invention is based on an approach to identify candidate genes involved in metabolic diseases, such as obesity and type 2 diabetes T2D through epigenetic mechanisms. This approach may also be utilized to identify genes involved in numerous diseases in addition to metabolic diseases.

Accordingly, in one embodiment, the invention provides a method for identifying a subject having or at risk of having a metabolic disease. The method includes identifying in the subject genetic markers correlating differentially methylated regions (DMRs) in the genome with genetic risk loci for the subject and comparing methylation patterns of the markers with a control sample from a subject not having the disease. In one embodiment, the disease is T2D. The method of the invention further includes analyzing adipose cells of the subject, wherein an inflammatory response is a factor associated with having or risk of having a metabolic disease, such as T2D.

In another embodiment, the invention also provides a method of treating a subject having or at risk of having a metabolic disease. The method includes increasing or decreasing gene expression of a genetic marker identified by the method of the invention based on an observation of hypomethylation or hypermethylation, respectively, of the marker, thereby treating the subject. In one embodiment, the genetic marker affects glucose utilization by a cell. In another embodiment, the genetic marker(s) is associated with obesity. In another embodiment, the genetic marker is one or more markers set forth in Table 2.

In another embodiment, the invention provides a method of providing a prognostic evaluation of a subject having or at risk of having a metabolic disease. The method includes analyzing one or more of the subject's genetic markers identified in the method of the invention prior to dietary and/or pharmaceutical intervention and following dietary and/or pharmaceutical intervention, and correlating a change in the genetic markers with a prognostic evaluation of the subject. In one embodiment, a decrease in expression of a marker previously up-regulated is correlated with improvement in the disease. In another embodiment, an increase in expression of a marker previously down-regulated is correlated with improvement in the disease.

In yet another embodiment, the invention provides a method for identifying a subject having or at risk of having a disease, such as for example, a metabolic disease, cancer, immune system disorder, cardiovascular disease, gastrointestinal disease or pulmonary disease. The method includes identifying in the subject one or more genetic markers correlating differentially methylated regions (DMRs) in the genome with genetic risk loci for the subject and comparing methylation patterns of the markers with a control sample from a subject not having the disease.

In another embodiment, the invention provides a method of determining a therapeutic regimen for a subject. The method includes identifying in the subject one or more genetic markers correlating differentially methylated regions (DMRs) in the genome with genetic risk loci for the subject and comparing methylation patterns of the markers with a control sample from a subject thereby assessing the therapeutic regimen for the subject.

### BRIEF DESCRIPTION OF THE DRAWINGS

FIGS. 1A-1B are graphical representations of data pertaining to genome-wide significant methylation changes related to diet-induced obesity in C57BL/6 mice.

FIGS. 2A-2B are graphical representations of data illustrating replication of mouse methylation changes in additional mice and associated gene expression changes.

FIGS. 3A-3B are graphical representations of data illustrating overlapping methylation changes in human and mouse adipose tissue.

FIGS. 4A-4B are diagrammatic representations of the interactions between epigenetically conserved and genetically associated genes implicated in this study.

FIG. 5A-5C are graphical representation of data illustrating overexpression and shRNA-mediated knockdown of selected genes in 3T3-L1 adipocytes.

FIG. 6 is a diagrammatic representation illustrating genetic characteristics of lean mice versus obese mice.

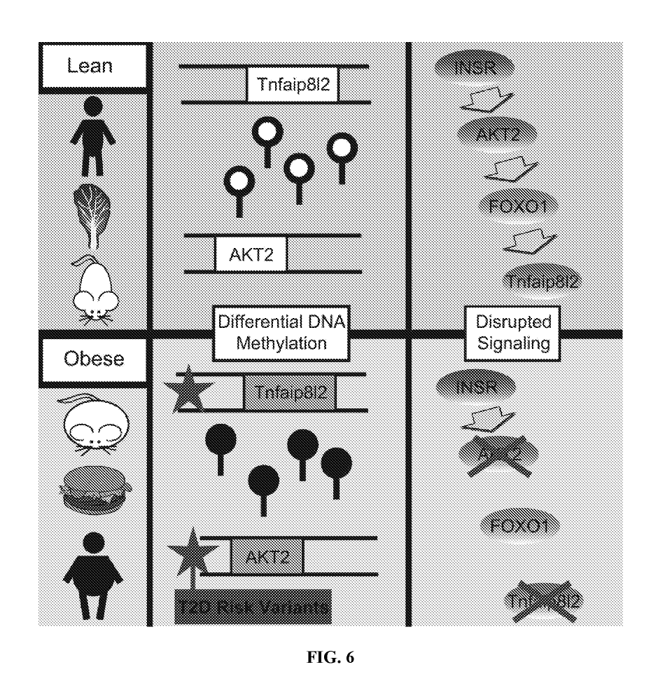

FIG. 7 is a series of graphical representations of data representing correlation of metabolic traits in a diet-induced obesity mouse model, related to FIG. 2.

FIGS. 8A-8B are graphical representations of data illustrating correlation of methylation and gene expression in mouse and human adipose tissue, related to FIG. 2.

FIGS. 9A-9C are graphical representations of data illustrating significance of methylation change overlap between mouse and human tissues, related to FIG. 3.

FIG. 10 is a graphical representation of data illustrating enrichment of connections between genes implicated by methylation and genome-wide significant GWAS genes, related to FIG. 4.

### DETAILED DESCRIPTION OF THE INVENTION

Using a functional approach to investigate the epigenetics of metabolic diseases, such as T2D, the invention methods are based on a combination of three lines of evidence (diet-induced epigenetic dysregulation in mouse, epigenetic conservation in humans, and T2D clinical risk evidence) to identify genes implicated in T2D pathogenesis through epigenetic mechanisms related to obesity. Beginning with dietary manipulation of genetically homogeneous mice, differentially DNA-methylated genomic regions were identified. These results were then replicated in adipose samples from lean and obese patients pre- and post-Roux-en-Y gastric bypass, identifying regions where both the location and direction of methylation change is conserved. These regions overlap with 27 genetic T2D risk loci, only one of which was deemed significant by GWAS alone. Functional analysis of genes associated with these regions revealed four genes with roles in insulin resistance, demonstrating the potential general utility of this approach for complementing conventional human genetic studies by integrating cross-species epigenomics and clinical genetic risk. While diabetes is provided as an illustrative example, it is believed that the analyses provided herein are applicable to epigenomics and clinical genetic risk for other metabolic diseases as well as cancer, immune system disorder, cardiovascular disease, gastrointestinal disease or pulmonary disease.

Before the present methods are described, it is to be understood that this invention is not limited to particular methods, and experimental conditions described, as such methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The present invention establishes an approach utilizing two species to identify candidate genes involved in obesity and T2D through epigenetic mechanisms. The experiments described herein examined the epigenetic consequences of a high-fat diet in a carefully controlled experimental mouse obesity setting. They then replicated across species-in humans-by analyzing adipose tissue from a cohort that both reproduces and reverses a phenotype similar to the obese mouse. The use of samples from the same subjects pre- and post-RYGB allows a human isogenic comparison of the effect of obesity-induced metabolic disturbances. This cross-species approach exploits the power of evolutionary selection, whose mechanisms have survived the 50 million year separation between mouse and human, in a more comprehensive manner than simple replication from human set to human set, and may better identify functionally important environmental targets. They lastly stratified these cross-species obesity-associated regions using genetic association data from a large genome-wide association study (GWAS) for T2D to more directly link the obesity-derived phenotypes with human T2D. As a result of this approach, the invention provides a method to identify genes with roles in insulin resistance, suggesting that this cross-species approach provides a powerful experimental system for identifying the genomic variation associated with common disease.

Accordingly, in one embodiment, the invention provides a method for identifying a subject having or at risk of having a metabolic disease. The method includes identifying in the subject genetic markers correlating differentially methylated regions (DMRs) in the genome with genetic risk loci for the subject and comparing methylation patterns of the markers with a control sample from a subject not having the disease.

A metabolic disease as used herein includes diseases that affect glucose utilization by a cell. Such diseases may include obesity, pre-diabetes, diabetes and the like. As illustrated in the Examples, the metabolic disease may be T2D. While the invention has identified genetic markers which are associated with metabolic disease, and in particular, obesity and diabetes, it will be understood by one in the art, the a similar approach may be taken to identify genetic markers associated with other types of diseases, for example, cancer, immune system disorder, cardiovascular disease, gastrointestinal disease and pulmonary disease.

As used herein, a “genetic marker” refers to, a nucleic acid molecule, such as a gene, gene promoter, or other region of a genome that may be observed and correlated with a disease. For example, a genetic marker may refer to a gene or other portion of a genome which may be assessed for methylation status. In this manner, a genetic marker includes a gene or differentially methylated region (DMR) of a genome. In various embodiments of the present invention, a genetic marker includes one or more genes or DMRs associated with one or more genes set forth in Table 2. For example, the genetic marker may be one or more genes or DMRs associated with Tcf712, As3mt, Etaa1, Tnfsf8, Plekho1, Tnfaip812, Akt2, Lhfp12, Mkl1, BC048644 (Car5a), Rgs3, Fgd3, Stau1, Tmcc3, Tbx3, Gstz1, Taok3, Bnip3, Dlst, Kcna3, Cln8, Cd37, Nfib, Pck1, Pcx, Hoxd3, Cd33 or Ev1. In a particular embodiment, the genetic marker includes at least Tcf712, or one or more of Mkl1, Plekho1 and Tnfaip812. For example, the genetic marker may include Tcf712 alone, Tcf712 in combination with one or more of Mkl1, Plekhol and Tnfaip812, or Tcf712 in combination with one or more of Tcf712, As3mt, Etaa1, Tnfsf8, Plekho1, Tnfaip812, Akt2, Lhfp12, Mkl1, BC048644 (Car5a), Rgs3, Fgd3, Stau1, Tmcc3, Tbx3, Gstzl, Taok3, Bnip3, Dlst, Kcna3, Cln8, Cd37, Nfib, Pck1, Pcx, Hoxd3, Cd33 or Ev1.

In another embodiment, the invention also provides a method of treating a subject having or at risk of having a metabolic disease. The method includes increasing or decreasing gene expression of a genetic marker identified by the method of the invention based on an observation of hypomethylation or hypermethylation, respectively, of the marker, thereby treating the subject.

Gene expression in the subject may be altered using various techniques as known in the art. For example, gene expression may be increased or decreased by administering an agent to the subject that effects gene expression. An agent, as used herein, is intended to include any agent capable of altering gene expression, for example, by altering the methylation status of a nucleic acid molecule. For example, an agent useful in any of the methods of the invention may be any type of molecule, for example, a polynucleotide, a peptide, a peptidomimetic, peptoids such as vinylogous peptoids, chemical compounds, such as organic molecules or small organic molecules, or the like. In various aspects, the agent may be a polynucleotide, such as DNA molecule, an antisense oligonucleotide or RNA molecule, such as microRNA, dsRNA, siRNA, stRNA, and shRNA.

In another embodiment, the invention provides a method of providing a prognostic evaluation of a subject having or at risk of having a metabolic disease. The method includes analyzing one or more of the subject's genetic markers identified in the method of the invention prior to dietary and/or pharmaceutical intervention and following dietary and/or pharmaceutical intervention, and correlating a change in the genetic markers with a prognostic evaluation of the subject. In one embodiment, a decrease in expression of a marker previously up-regulated is correlated with improvement in the disease. In another embodiment, an increase in expression of a marker previously down-regulated is correlated with improvement in the disease.

In yet another embodiment, the invention provides a method for identifying a subject having or at risk of having a disease, such as, a metabolic disease, cancer, immune system disorder, cardiovascular disease, gastrointestinal disease or pulmonary disease. The method includes identifying in the subject one or more genetic markers correlating differentially methylated regions (DMRs) in the genome with genetic risk loci for the subject and comparing methylation patterns of the markers with a control sample from a subject not having the disease.

In another embodiment, the invention provides a method of determining a therapeutic regimen for a subject. The method includes identifying in the subject one or more genetic markers correlating differentially methylated regions (DMRs) in the genome with genetic risk loci for the subject and comparing methylation patterns of the markers with a control sample from a subject thereby assessing the therapeutic regimen for the subject.

In the present invention, the subject is typically a human but also can be also be any non-human mammal or other classes, including, but not limited to, a dog, cat, rabbit, cow, bird, rat, horse, pig, or monkey.

In the various methods of the invention, methylation status of a nucleic acid molecule, such as a gene, or a region of a genome identified as a DMR and correlated with a disease is assessed. In various aspects of the invention a genetic marker such as a gene or DMR may be hypermethylated or hypomethylated as compared to a control. Hypomethylation is present when there is a measurable decrease in methylation . In some embodiments, a marker can be determined to be hypomethylated when less than 50% of the methylation sites analyzed are not methylated. Hypermethylation is present when there is a measurable increase in methylation. In some embodiments, a marker can be determined to be hypermethylated when more than 50% of the methylation sites analyzed are methylated. Methods for determining methylation states are provided herein and are known in the art. In some embodiments methylation status is converted to an M value. As used herein an M value, can be a log ratio of intensities from total (Cy3) and McrBC-fractionated DNA (Cy5): positive and negative M values are quantitatively associated with methylated and unmethylated sites, respectively. M values are calculated as described in the Examples. In some embodiments, M values which range from −0.5 to 0.5 represent unmethylated sites as defined by the control probes, and values from 0.5 to 1.5 represent baseline levels of methylation.

Numerous methods for analyzing methylation status of a gene are known in the art and can be used in the methods of the present invention to identify either hypomethylation or hypermethylation. In some embodiments, bisulfite pyrosequencing, which is a sequencing-based analysis of DNA methylation that quantitatively measures multiple, consecutive CpG sites individually with high accuracy and reproducibility, may be used. Exemplary primers for such analysis are set forth in Tables 3 and 4.

It will be recognized that depending on the site bound by the primer and the direction of extension from a primer, that the primers listed above can be used in different pairs. Furthermore, it will be recognized that additional primers can be identified within the DMRs, especially primers that allow analysis of the same methylation sites as those analyzed with primers that correspond to the primers disclosed herein.

Altered methylation can be identified by identifying a detectable difference in methylation. For example, hypomethylation can be determined by identifying whether after bisulfite treatment a uracil or a cytosine is present a particular location. If uracil is present after bisulfite treatment, then the residue is unmethylated. Hypomethylation is present when there is a measurable decrease in methylation.

In an alternative embodiment, the method for analyzing methylation can include amplification using a primer pair specific for methylated residues within a nucleic acid molecule. In these embodiments, selective hybridization or binding of at least one of the primers is dependent on the methylation state of the target DNA sequence (Herman et al., Proc. Natl. Acad. Sci. USA, 93:9821 (1996)). For example, the amplification reaction can be preceded by bisulfite treatment, and the primers can selectively hybridize to target sequences in a manner that is dependent on bisulfite treatment. For example, one primer can selectively bind to a target sequence only when one or more base of the target sequence is altered by bisulfite treatment, thereby being specific for a methylated target sequence.

Other methods are known in the art for determining methylation status, including, but not limited to, array-based methylation analysis and Southern blot analysis.

Methods using an amplification reaction, for example methods above for detecting hypomethylation or hyprmethylation of one or more DMRs, can utilize a real-time detection amplification procedure. For example, the method can utilize molecular beacon technology (Tyagi et al., Nature Biotechnology, 14: 303 (1996)) or Taqman™ technology (Holland et al., Proc. Natl. Acad. Sci. USA, 88:7276 (1991)).

Also methyl light (Trinh et al., Methods 25(4):456-62 (2001), incorporated herein in its entirety by reference), Methyl Heavy (Epigenomics, Berlin, Germany), or SNuPE (single nucleotide primer extension) (see e.g., Watson et al., Genet Res. 75(3):269-74 (2000)) Can be used in the methods of the present invention related to identifying altered methylation of DMRs.

The degree of methylation in the DNA associated with the DMRs being assessed, may be measured by fluorescent in situ hybridization (FISH) by means of probes which identify and differentiate between genomic DNAs, associated with the DMRs being assessed, which exhibit different degrees of DNA methylation. FISH is described, for example, in de Capoa et al. (Cytometry. 31:85-92 (1998)) which is incorporated herein by reference. In this case, the biological sample will typically be any which contains sufficient whole cells or nuclei to perform short term culture. Usually, the sample will be a sample that contains 10 to 10,000, or, for example, 100 to 10,000, whole cells.

Additionally, as mentioned above, methyl light, methyl heavy, and array-based methylation analysis can be performed, by using bisulfite treated DNA that is then PCR-amplified, against microarrays of oligonucleotide target sequences with the various forms corresponding to unmethylated and methylated DNA.

To examine DNAm on a genome-wide scale, comprehensive high-throughput array-based relative methylation (CHARM) analysis, which is a microarray-based method agnostic to preconceptions about DNAm, including location relative to genes and CpG content may be utilized. The resulting quantitative measurements of DNAm, denoted with M, are log ratios of intensities from total (Cy3) and McrBC-fractionated DNA (Cy5): positive and negative M values are quantitatively associated with methylated and unmethylated sites, respectively. For each sample, ˜4.6 million CpG sites across the genome of a may be analyzed. In embodiments, methylation status is determined according to the method set forth in Irizarry et al. (Genome Res. 18:780-790 (2008)) or Ladd-Acosta et al. (Current Protocols in Human Genetics 20.1.1-20.1.19 (2010)), both of which are incorporated herein by reference in their entireties.

In various embodiments, the determining of methylation status in the methods of the invention is performed by one or more techniques selected from the group consisting of a nucleic acid amplification, polymerase chain reaction (PCR), methylation specific PCR, bisulfite pyrosequenceing, single-strand conformation polymorphism (SSCP) analysis, restriction analysis, microarray technology, and proteomics. As illustrated in the Examples herein, analysis of methylation can be performed by bisulfite genomic sequencing. Bisulfite treatment modifies DNA converting unmethylated, but not methylated, cytosines to uracil. Bisulfite treatment can be carried out using the METHYLEASY™ bisulfite modification kit (Human Genetic Signatures).

In the various methods of the invention, genetic markers can be identified from a sample from the subject. A sample can be taken from any tissue that is susceptible to disease. A sample may be obtained by surgery, biopsy, swab, stool, or other collection method. In some embodiments, the sample is derived from blood, adipose tissue, pancreatic tissue, liver tissue, serum, urine, saliva, cerebrospinal fluid, pleural fluid, ascites fluid, sputum, stool, skin, hair or tears.

The following examples are provided to further illustrate the advantages and features of the present invention, but are not intended to limit the scope of the invention. While they are typical of those that might be used, other procedures, methodologies, or techniques known to those skilled in the art may alternatively be used.

### EXAMPLE I

### Mouse-Human Experimental Epigenetic Analysis Unmasks Dietary Targets and Genetic Liability for Diabetic Phenotypes

The inventors established an approach utilizing two species to identify candidate genes involved in obesity and Type 2 Diabetes (T2D) through epigenetic mechanisms. The inventors first examined the epigenetic consequences of a high-fat diet in a carefully controlled experimental mouse obesity setting. The inventors then replicated across species (in humans) by analyzing adipose tissue from a cohort that both reproduces and reverses a phenotype similar to the obese mouse. The use of samples from the same subjects pre- and post-RYGB allows a human isogenic comparison of the effect of obesity-induced metabolic disturbances. This cross-species approach exploits the power of evolutionary selection, whose mechanisms have survived the 50 million year separation between mouse and human, in a more comprehensive manner than simple replication from human set to human set, and may better identify functionally important environmental targets. The inventors lastly stratified these cross-species obesity-associated regions using genetic association data from a large genome-wide association study (GWAS) for T2D to more directly link the obesity-derived phenotypes with human T2D. As a result of this approach, the inventors are able to identify four genes with roles in insulin resistance, suggesting that this cross-species approach provides a powerful experimental system for identifying the genomic variation associated with common disease.

The following experimental protocols and materials were utilized.

Mouse Sample Preparation

All animal protocols were approved by the Institutional Animal Care and Use Committee of The Johns Hopkins University School of Medicine. Male C57BL/6 mice were purchased from Charles River and housed in polycarbonate cages on a 12-h light-dark photocycle with ad libitum access to water and food. Mice were fed a high-fat diet (HFD; 60% kcal derived from fat, Research Diets; D12492) or the matched control low-fat diet (LFD; 10% kcal derived from fat, Research Diets; D12450B). Diet was provided for a period of 12 weeks, beginning at 4 weeks of age. At termination of the study, animals were fasted overnight and euthanized; tissues were collected, snap frozen in liquid nitrogen, and kept at −80° C. until analysis.

Intraperitoneal Glucose and Insulin Tolerance Tests

Cohorts of mice (between 20 and 24 weeks of age) were injected with glucose (1 g/kg body weight) or insulin (0.8 units/kg for LFD-fed mice, 1.2 units/kg for HFD-fed mice). Animals were fasted overnight (16 h) prior to the glucose tolerance test. For the insulin tolerance test, food was removed 2 h prior to insulin injection. Serum samples were collected by using microvette CB 300™ (Sarstedt). Glucose concentrations were determined at time of blood collection with a glucometer (BD Biosciences). Six blood samples were collected at sequential timepoints after injections.

Mouse Hepatocyte Isolation

A protocol for primary hepatocyte isolation was adapted from previously published methods. Mice were anesthetized and a catheter was inserted into the vena cava. The portal vein was then cut to allow liver-specific perfusion. Mice were then perfused with PBS, followed by 100 ug/mL Type I Collagenase (BD Biosciences) at a rate of 5 ml/min for 10 min. The liver was then removed and dissociated by straining through a 70 m pore nylon cell strainer (BD Falcon). The cells were then spun down and resuspended in William's Medium E™ (Cellgro). Primary hepatocytes were then isolated by gradient distribution via centrifugation of the resuspension in a cold Percoll™ (GE healthcare) solution. Verification of primary hepatocyte purity was assessed via quantitative real-time PCR for hepatocyte-specific genes compared to markers for endothelial and immune cells. The inventors observed >90% hepatocyte purity based on gene expression.

Mouse Primary Adipocyte Isolation

Mature adipocytes were isolated from mouse fat pads as previously described. Briefly, fat pads were finely chopped using scissors. Tissue was then dissociated in 2 mg/gram tissue Type II Collagenase (Sigma) in KRH buffer. The digestion was stopped by adding 10% FBS (Atlantic Biologicals) to the mixture and cells were filtered through 100 μm pore nylon cell strainers (BD Falcon). The cells were then separated out by transferring the upper phase of cells to a new tube and washing with 5 mL of KR Buffer. The wash and resuspension was repeated 3 times and mature adipocytes were collected. Verification of mature adipocyte purity was assessed via quantitative real-time PCR for adipose-specific genes compared to markers for endothelial and immune cells. The inventors observed >95% adipocyte purity based on gene expression.

Pancreatic Islet Isolation

Pancreatic islets used for CHARM were isolated as previously described. For the pancreatic islets used in the replication set, whole pancreases were obtained from high-fat-fed and lowfat-fed mice, stained for insulin using the Anti-Insulin+Proinsulin antibody [D3E7]™ (Biotin) (ab20756) (Abcam, Mass., USA) kit, cryosectioned into 8 μm sections, and then laser-capture microdissection was used to isolate pancreatic islets (PALM Microbeam, Carl Zeiss, N.C., USA).

3T3-L1 Transduction and Transfection

3T3-L1 cells were transducted with Sigma Mission™ lentiviral particles and transfected with overexpression plasmids using Lipofectamine™ 3000 (Life Technologies) as per the respective manufacturers' protocols. Cells were plated at 60% confluency and incubated for 18 hours in a humidified incubator. Media was removed and replaced by Opti-MEM™ (Invitrogen) with 8 μg/ml Hexadimethrine Bromide (Sigma-Aldrich). Fifteen μl lentiviral particles were added and the plates were incubated for 18 hours in a humidified incubator. Media was then removed and replaced, and on the following day media containing 10 μg/ml puromycin (Sigma Aldrich) was added and the cells were cultured in puromycin thereafter.

3T3-L1 cells were transfected with overexpression plasmids using Lipofectamine™ 3000 (Life Technologies) as per the manufacturer's protocol. Cells were plated at 60% confluency and incubated for 18 hours in a humidified incubator. Lipofectamine™ 3000 (1.5 μl per well containing cells) was diluted and mixed in 50 μl Opti-MEM medium (Invitrogen). At the same time, 4 μg plasmid DNA was diluted in 50 μl Opti-MEM with 2 μ P3000™ reagent and mixed. The diluted Lipofectamine™ and plasmid DNA were then mixed, incubated for 5 min at room temperature, and distributed onto the plated cells. After 24 hours incubation, the media was replaced with growth media. After 48 hours, 500 μg/ml Geneticin Selective Antibioti™ (G418 Sulfate, Life Technologies) was added, and the cells were maintained in geneticin thereafter.

Lentiviral particles used: Tmcc3 (TRCN0000126784, Sigma Aldrich), Gstz 1 (TRCN0000103080, Sigma Aldrich), MISSION® TRC2 pLKO.5-puro Non-Mammalian shRNA Control Transduction Particles™ (Control, SHC202V, Sigma Aldrich).

Overexpression plasmids used: Mkl1 (MC202660, Origene), Plekhol (MC210507, Origene), Tnfaip812 (MC203559, Origene), Cloning vector PCMV6-Kan/Neo (Control, PCMV6KN, Origene).

Cell Culture and Glucose Uptake Assay

3T3-L1 cell lines (ATCC) were maintained in Dulbecco's Modified Eagle Medium (Invitrogen) supplemented with 10% FBS (Invitrogen), and 10 μg/ml puromycin and 500 μg/ml geneticin (G418) as selective antibiotics for the knock-down and overexpression lines, respectively. Two days after confluence, differentiation of the knock-down lines was induced by incubation with MDI medium (4 μg/ml insulin, 0.5 mM Methylisobutylxanthine (IBMX), 1.0 μM dexamethasone) for 2 days and 4 μg/ml insulin for 5 days. Differentiation of the over-expression lines was induced with MDI medium and 1 μM rosiglitazone for 3 days and 4 μg/ml insulin for 3 days. After another 3-5 days of incubation with maintenance medium, 80%-100% differentiation was shown by lipid droplet accumulation in the cells. Glucose uptake assays were performed on differentiated knock-down and over-expression lines. After 2 h of incubation in serum-free DMEM, they were washed twice in pre-warmed PBS and placed in HEPES buffered saline solution (25 mM HEPES, pH 7.4, 120 mM NaCl, 5 mM KCl, 1.2 mM MgSO4, 1.3 mM CaCl2, 1.3 mM KH2PO4, and 0.5% BSA) containing 10 nM or 100 nM insulin for 20 min. Then, 0.5 μCi/well 2-deoxy-D-[3H]glucose (Moravek) was added for 5 min. The reactions were terminated by two ice-cold PBS washes. Cells were then incubated for 10 min with whole cell lysis buffer (20 mM Tris-HCl, 150 mM NaCl, 1 mM EDTA, 0.5% NP-40, and 10% glycerol). The lysates were transferred to scintillation vials containing Ecoscint™ scintillation fluid (National Diagnostics) and counted with a Beckman Coulter counter (model LS 6000SC).

Human Sample Surgery and Subcutaneous Adipose Tissue Biopsies

A standard laparoscopic RYGB with a 1 m Roux limb was performed. The patients were weight stable and not subjected to a preoperative weight loss period. Subcutaneous abdominal adipose biopsies (50-100 mg) were obtained from the obese and non-obese (normal weight) subjects. Biopsies were obtained at the beginning of RYGB surgery (obese subjects) or elective laparoscopic cholecystectomy (lean subjects) after the induction of general anesthesia. Only non-glucose-containing intravenous solutions were administered before the biopsy was taken during RYGB or elective cholecystectomy surgery after an overnight fast. Biopsies taken from the obese subjects 6 months after RYGB surgery were obtained under local anesthesia (5 mg/ml of lidocaine hydrochloride) in the morning after an overnight 12 hour fast from the same surgical incision as the initial biopsy. Biopsy samples for DNA analysis were immediately frozen and stored in liquid nitrogen until analysis. Fat and liver biopsies were obtained at the beginning of RYGB surgery (obese subjects) or elective laparoscopic cholecystectomy (lean subjects) after the induction of general anesthesia.

CHARM DNA Methylation Analysis

Genomic DNA from all samples was purified with the MasterPure™ DNA purification kit (Epicentre) following the manufacturer's protocol. Genomic DNA (1.5-2 μg) was fractionated with a Hydroshear Plus™ (Digilab), digested with McrBC, gel-purified, labeled and hybridized to a CHARM microarray as described. The mouse CHARM 2.0™ array used in the analysis now includes 2.1 million probes, which cover 5.2 million CpGs arranged into probe groups (where consecutive probes are within 300 bp of each other) that tile regions of at least moderate CpG density. The human CHARM 3.0™ array now includes 4.1 million probes, which cover 7.5 million CpGs. These arrays include all annotated and non-annotated promoters and microRNA sites on top of the features that are present in the original CHARM method. The inventors dropped 7 human arrays with <80% of their probes above background intensities, resulting in 11 pre-surgery obese samples, 8 post-surgery obese samples, and 8 lean samples that underwent DNA methylation analysis. The design specifications are freely available on the World Wide Web at rafalab.jhu.edu. The inventors then removed sex chromosomes to improve the batch correction methods.

Subsequent pre-processing, normalization and correction for batch effects were performed as previously described. Briefly, the inventors applied a “bump hunting” approach which involves a) performing linear regression at each probe, comparing DNA methylation levels versus a covariate of interest (e.g. high-versus low-fat diet), adjusting for surrogate variables, b) smoothing the regression coefficient for the covariate of interest across nearby probes and c) thresholding these smoothed regression coefficients across all probe groups, which forms differentially methylated regions (DMRs) representing adjacent probes with statistics above the threshold. Each DMR is summarized by its “area”, or the sum of the adjacent statistics above the threshold. The inventors used the 99.9th percentile of the smoothed statistics for each respective species, tissue and trait comparisons bump hunting analysis. Statistical significance was assessed via linear model bootstrapping, retaining surrogate variables, followed by bump hunting, which approximates full permutation (e.g. permuting trait, recalculating surrogate variables, then bump hunting) using much less computational time.

Bisulfite Pyrosequencing

Genomic DNA (gDNA, 200 ng) from each replication sample was bisulfite treated using the EZ DNA Methylation-Gold™ Kit (Zymo research) according to the manufacturer's protocol. Bisulfite-treated gDNA was PCR amplified using nested primers, and DNA methylation was subsequently determined by pyrosequencing with a PSQ HS96 (Biotage) as previously reported. Artificially methylated control standards of 0, 25, 50, 75 and 100% methylated samples were created using mixtures of purified and Sssl-treated whole genome amplified (REPLI-g™ amplification kit, Qiagen) Human Genomic DNA: Male™ (Promega). Pyrosequencing primers are shown in Table 3.

Quantitative PCR Analysis

Validated primers for all genes were taken from PrimerBank™ and synthesized by Integrated DNA Technologies (Coralville, Iowa, USA). RNA was extracted with Trizol reagent (Life Technologies, Carlsbad, Calif., USA), cDNA was created with Quantitect Reverse Transcriptase Kit™ (Qiagen, Venlo, Netherlands), and quantitative-PCR was performed with Fast SYBR Green™ (Applied Biosystems, Foster City, Calif., USA) on a 7900HT Fast Real-Time PCR™ system (Applied Biosystems, Foster City, Calif., USA). RNA levels were normalized to same-sample 18S RNA levels. Quantitative PCR primers are shown in Table 4.

GO Annotation

The inventors analyzed GO annotation using the GOrilla™ tool. Enrichment was calculated by comparing genes identified from the analysis to a background of all genes detectable on the appropriate array.

Whole-Genome Gene Expression Analysis

Whole genome gene expression data for mouse and human analogues of the study was downloaded from GEO. The mouse data was already pre-processed, and the human data was pre-processed using Robust Multi-array Averaging™ (RMA) from the Affy R™ library (Bioconductor). The gene expression data was then matched against the DMRs closest to corresponding genes, the log fold change (logFC) of the gene expression was plotted against the average value of the smoothed effect estimate within the DMR, and p-values were generated using t-tests based on Pearson's correlation coefficient.

Enrichment Between Human and Mouse DMRs

The liftOver™ tool from the UCSC genome browser transformed the coordinates from the human DMRs from the hg19 human genome to the mm9 mouse genome, as implemented in the rtracklayer Bioconductor™ package. The locations of the 249,094 probe groups on the human CHARM array were also lifted over to serve as the natural background for enrichment, of which 214,646 (86.2%) had any analogous sequence in mouse, and a further 109,234 (50.9%) were within 5 kb of a mouse CHARM probe group. For each pair of DMR lists, one from the two lifted-over human DMRs and another from the 25 mouse trait DMRs (see Table S1 of Feinberg et al. (Cell Metabolism 21(1):138-149 (2015)) publicly available on the World Wide Web at sciencedirect.com/science/article/pii/S1550413114005658, which is incorporated herein by reference in its entirety; Table S1shows the results of CHARM analysis for five assayed mouse tissues against five measured metabolic phenotypes of diet, fasting glucose, mouse weight, glucose tolerance test and insulin tolerance test and is related to Table 1 herein), the inventors calculated the number of DMRs at given within specific p-value significance levels, and also the number that overlapped within 5kb across species. Enrichment tests were chi-squared tests based on the number of species-overlapping significant DMRs, then DMRs only significant within each species, and finally the number of lifted probe group (of the 109,234) that were not significant in either species (which creates a 2×2 table of the number significant in both species, significant in just human, significant in just mouse, and significant in neither species). This is analogous to creating a Venn diagram between significant human and mouse DMRs.

Cross-Species Statistical Analysis

The inventors combined significant adipocyte mouse DMRs (at FDR <5%) across the five traits (glucose, GTT, ITT, weight, and diet) by retaining the maximal coordinates over overlapping cross-trait DMRs resulting in 625 independent DMRs associated with at least 1 trait in adipocytes in mouse. These regions were lifted over from the mouse mm9 genome build to the human hg19 genome build as implemented in the rtracklayer Bioconductor package (Lawrence et al., 2009). These DMRs were annotated to the nearest human charm probe group based on the annotation within 5 kb. The inventors then computed a difference and corresponding p-value in obese versus lean and then in obese humans pre-versus post RYGB surgery using linear regression, and retained the minimum p-value, number of probes with p <0.05, and the slope at the smallest p-value, within each of the mapped DMRs.

DIAGRAM GWAS Analysis

The inventors integrated GWAS results into the 497 mouse-human DMRs by obtaining publicly available results from the DIAGRAM meta-analysis (available on the World Wide Web at diagram-consortium.org/downloads.html; Stage 1 GWAS: Summary Statistics download) with coordinates in genome build hg18. The separate GWAS studies that make up this meta-analysis have each been corrected for population structure differences, and the meta-analysis summary statistics (e.g. test statistics and p-values per SNP) are available for public download. The inventors then generated regions of high genotypic correlation by taking all SNP rs numbers with p <0.01 (n=39,081) passing them through the SNAP tool using CEU 1000 Genomes Pilot 1 data (Johnson et al., 2008), obtaining proxy SNPs with R2 >0.8 (n=167,055 unique proxies), and recording the coordinate range of the proxies for each SNP. Overlapping per-SNP risk regions were merged if overlapping (n=7,946 genotypic risk regions) and the smallest p-value across all merged SNPs represented the p-value for the genotypic risk region. These genotypic regions were lifted over to hg19 coordinates for cross-species analysis as described above. The inventors estimated the variance in disease susceptibility based on disclosed algorithms using 1000 genomes-derived risk allele frequencies and assuming a disease prevalence of 8% for a given collection of risk SNPs.

The inventors assessed potential enrichment between DMRs and the GWAS results using two complementary approaches. The first approach assessed the enrichment in genome location between DMRs and the LD blocks from the GWAS. This permutation-based enrichment test is performed on two lists of genomic regions (e.g. chr:start-end) that assesses the degree of overlap relative to the background genome. At a given GWAS p-value cutoff, the inventors counted the proportion of GWAS signals that overlapped at least 1 DMR, and then generated background overlap by resampling the same number of GWAS regions (and the same length distribution) 10,000 times from the mappable genome (e.g. the genome after removing coordinates corresponding to telomeres, centromeres and other gaps present in genome build hg19, available from UCSC). Empirical p-values for enrichment were calculated by counting the number of null proportions that were greater than the observed proportion. R code is available on GitHub™.

The second approach assessed enrichment in gene symbols based on all genes directly connected (one-step) to genes linked to T2D with genome-wide significance by the DIAGRAM meta-analysis based on regulatory networks generated using Qiagen's Ingenuity IPA™. These sets (also known as interaction networks in Ingenuity) were able to be generated for 57 out of 59 genome-wide significant genes. Full interaction networks were not able to be retrieved for the remaining two genes, and these were excluded from the analysis. These interaction networks then had chemicals, groups, complexes and miRNAs filtered in order to limit the potential interacting partners to genes and protein products.

The inventors computed whether genes overlapping obesity-related DMRs were more likely to be associated with GWAS genes and their interaction networks. The inventors first removed DMRs that were not within 10 kb of a RefSeq gene, leaving 244 and 471 obesity-related DMRs in islet and adipose tissue respectively (from 312 and 576). Then the inventors counted the number of GWAS-associated genes and their directly connected partners in the genes containing DMRs. This procedure was also performed after the cross-species conservation filtering step described above, leaving 44 and 146 conserved obesity-related DMRs overlapping genes. The inventors obtained statistical significance based on a resampling analysis, where the inventors resampled the same number of probes groups 100,000 times from all probes groups mapped to human genes on the mouse CHARM design by: 1) lifting the range of the coordinates of each probe group to hg19, 2) removing poorly lifted probes groups defined as greater than 1.5 times the longest (in bp) original probe group prior to lifting over, 3) assigning the nearest human gene to each lifted probe group, and 4) dropping lifted probes groups not within 10 kb of a human RefSeq gene. The inventors counted the number of GWAS signals or their directly connected partners that overlapped the resampled genes in each iteration, and calculated an empirical p-value based on this null distribution. This procedure was therefore performed four times, for both adipose and islet DMRs with and without filtering for cross-species conservation.

Data Availability

Both raw and processed microarray data has been uploaded to GEO, the Gene Expression Omnibus™, as series record GSE63981.

Results

Alterations in DNA Methylation in Mouse Adipocytes Produced by High-Fat Diet

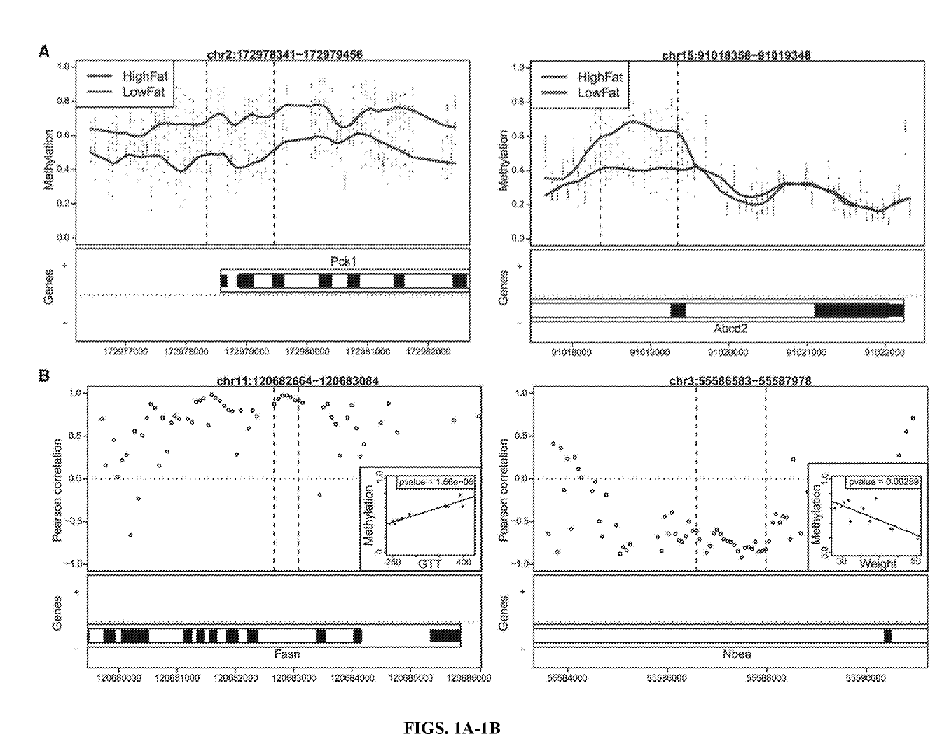

To detect DNA methylation differences, the inventors used the comprehensive high-throughput array-based relative methylation (CHARM) method, which in its current form can assay over 5 million CpG sites in mouse and 7.5 million CpG sites in human. In 12 adipocyte samples extracted from mouse adipose tissue, the inventors found 232 differentially methylated regions (DMRs) correlated with diet status (Table 1). As an example, when comparing adipocytes from high-fat-fed mice versus low-fat-fed mice, the inventors found hypermethylation overlying the promoter of phosphoenolpyruvate carboxykinase 1 (Pck1, FIG. 1A). PEPCK, the product of Pck1, catalyzes a rate-limiting step in gluconeogenesis, is essential for lipid metabolism in adipose tissue, is known to be regulated by insulin, and has been linked to lipodystrophy and obesity in mice.

FIGS. 1A-1B are graphical representations of data illustrating genome-wide significant methylation changes related to diet-induced obesity in C57BL/6 mice. In FIG. 1A, two genome-wide significant DMRs are hypermethylated in adipocytes purified from mice raised on a high-fat diet. Each point represents the methylation level in adipocytes from an individual mouse at a specific probe, with smoothed lines representing group methylation averages. These points are colored blue for lean mice and red for obese mice.

In FIG. 1B, body weight (grams) and glucose tolerance (AUC) are associated with methylation in adipocytes at genome-wide significant levels. Each point in the top panels represents one probe, with the y axis representing the Pearson correlation coefficients of the probes with the analyzed phenotype. Dotted lines represent the extent of the DMR as generated automatically via CHARM. The bottom panels display gene location information for the chromosomal coordinates on the x axis.

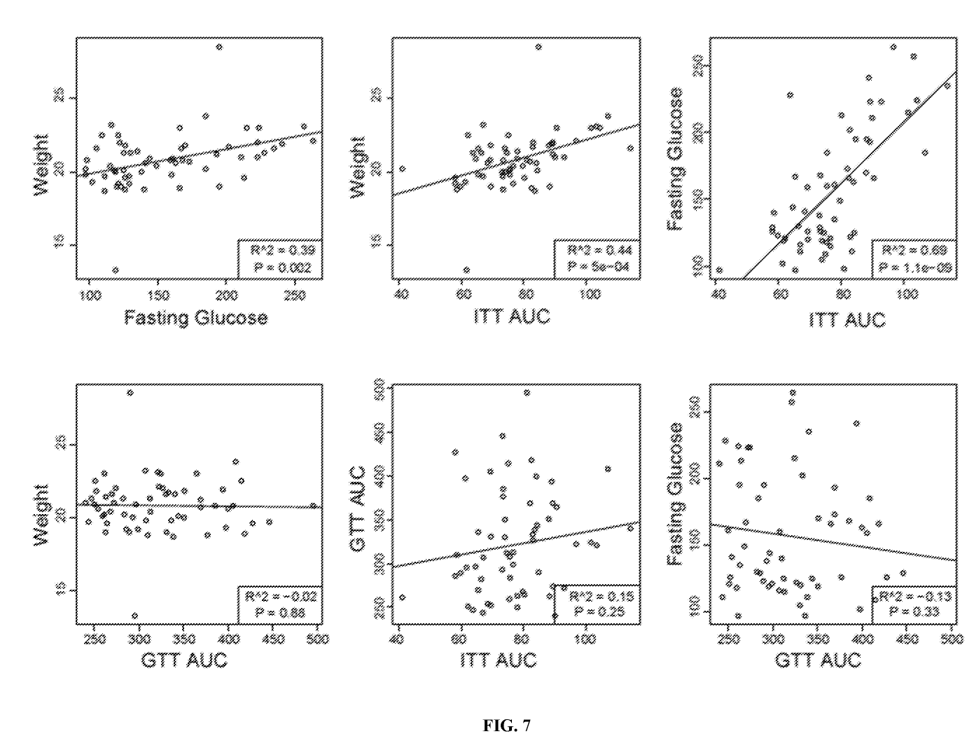

In addition to the high-fat versus low-fat analysis, even more DMRs were detected when analyzing methylation differences related to the metabolic phenotypes of body weight, fasting glucose, and insulin and glucose tolerance test area-under-curve (ITT/GTT AUC) values (see Table 1 herein and Table S1 of Feinberg et al. (Cell Metabolism 21(1):138-149 (2015))). One example of a mouse GTT-associated DMR is in the Fasn gene, which produces fatty acid synthase. Most DMRs found were significantly associated with more than one trait, which is not entirely unexpected as the phenotypes themselves are highly correlated (FIG. 7).

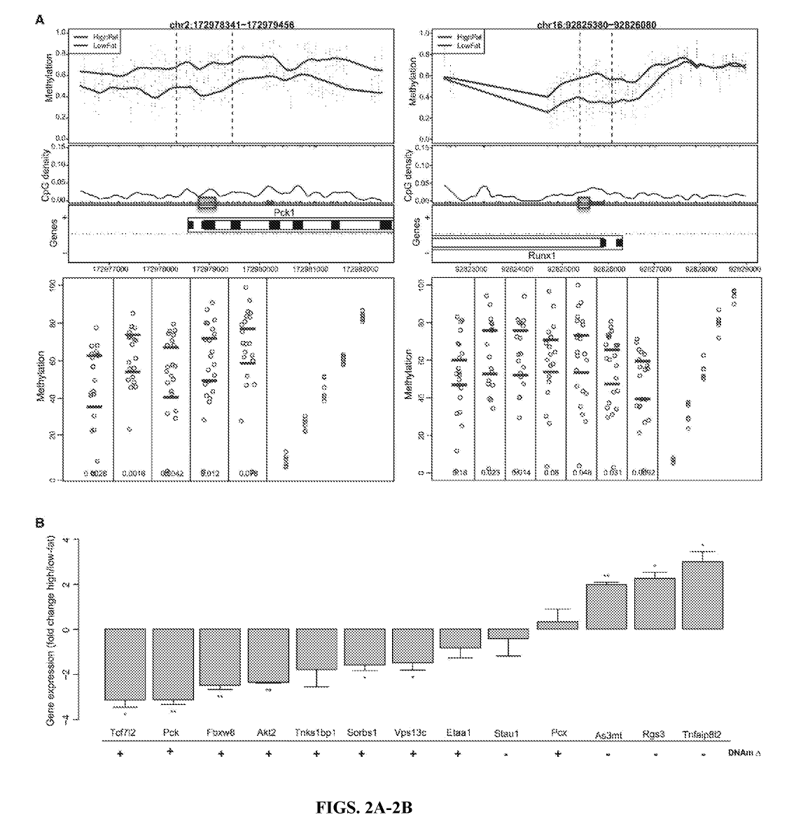

FIG. 7 is a series of graphical representations of data representing correlation of metabolic traits in a diet-induced obesity mouse model, related to FIG. 2. The Figure shows correlations between the mouse traits observed over time. Mouse weight, fasting glucose levels (collected at the time of glucose tolerance test), and insulin tolerance test and glucose tolerance test area-under-thecurve scores are plotted and correlated against each other. Correlation coefficients and p-values for the linear models are shown in the inserts.

The inventors additionally examined DNA methylation in pancreatic islets purified from whole mouse pancreata and hepatocytes extracted from mouse liver tissue. The inventors found significant correlations between methylation and mouse diet and weight in pancreatic islets and correlations between methylation and weight and ITT in hepatocytes (see Table S1 of Feinberg et al. (Cell Metabolism 21(1):138-149 (2015))).

Pooling tissues together and surveying for DNA methylation changes in common across tissues yielded no significant results.

Gene Ontology for Mouse DMRs

The inventors implemented gene set analyses to assess the overall biological importance of the DNA methylation changes the inventors observed in mouse adipocytes. The genome-wide significant adipocyte DMRs were near genes that were significantly overrepresented in lipid metabolic and immune/inflammatory pathways compared to the background list of genes represented on the array, with enrichment q values <9.7×10⁻³ (Table 5). Examining hyper- and hypomethylated DMRs separately in high-fat-fed obese mice, the inventors observed that the metabolic pathway enrichment was derived from genes near hypermethylated DMRs, while the inflammatory pathway enrichment was present mainly in genes near hypomethylated DMRs.

Inflammatory and immune-related systems are known to be upregulated in adipocytes specifically in both obesity and T2D. Similarly, recent work has shown adipose de novo lipogenesis downregulation associated with metabolic dysfunction. These pathways, however, have not previously been shown to be significantly associated with methylation changes in a diet-induced obesity phenotype.

Methylation Replication in Mice and Associated Gene Expression Studies

The inventors then tested for replication of the methylation results at nine DMRs in adipocytes and three DMRs in pancreatic islets in an independent set of 18 mice (see FIG. 2A herein and Table S3 of Feinberg et al. (Cell Metabolism 21(1):138-149 (2015)) publicly available on the World Wide Web at sciencedirect.com/science/article/pii/S1550413114005658, which is incorporated herein by reference in its entirety; Table S3 contains the results of pyrosequencing assays to replicate the CHARM results in separate samples). The 625 genome-wide significant adipocyte DMRs have FDR q values ranging from 0.004 to 0.05. In order to determine whether the results would replicate throughout this range, the inventors examined a subset of DMRs with levels of statistical significance that spanned from the most significant to just below the 0.05 cutoff. Mice used in the replication set were also reared on a high-fat diet but were separate from those used for CHARM. Nine mouse adipocyte DMRs were assayed by bisulfite pyrosequencing. Eight of these regions had at least one CpG showing significant differential methylation in the same direction as detected by CHARM.

FIGS. 2A-2B are graphical representations of data illustrating replication of mouse methylation changes in additional mice and associated gene expression changes. In FIG. 2A, methylation changes observed after CHARM analysis at two genome-wide significant DMRs are replicated using bisulfite pyrosequencing. Red boxes indicate CpGs assayed in pyrosequencing. For the lower pyrosequencing plots, the y axis represents methylation, and individual CpGs are plotted along the x axis. Purple dots represent control DNA artificially methylated to have 0%, 25%, 50%, 75%, and 100% methylation.

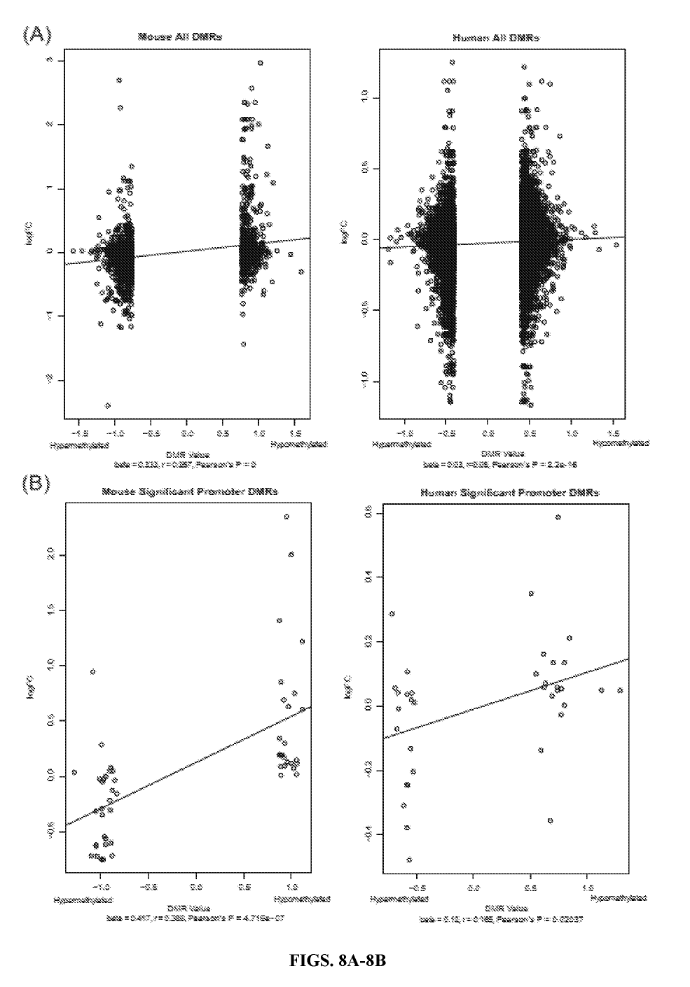

In FIG. 2B, gene expression changes for genes near genome-wide significant mouse adipocyte DMRs. RNA levels were normalized to same-sample 18S RNA measurements and are displayed as (C_{T} [high-fat samples]-C_{T} [low-fat samples]). Error bars represent standard error of the C_{T} differences between groups. *p <0.05, **p <0.005. The direction of the genome-wide significant CHARM DMR closest to the gene is denoted below the gene names; +and−represent regions hyper- or hypomethylated in the high-fat samples, respectively. See also FIG. 8 for whole-genome gene expression correlations and Table 6 and Table 7 for pyrosequencing and tissue purification, respectively.

Although these were fractionated cells under investigation, to further ensure that the results were not due to cell-type shifts in the high-fat-fed obese mice resulting from the infiltration of immune cells into adipose tissue, the inventors used quantitative PCR (qPCR) to characterize the expression of multiple macrophage- and adipocyte-specific markers in the purified adipocyte samples from low-fat-fed and high-fat-fed mice. The inventors saw no significant change in the levels of expression of the macrophage (inflammatory) markers F4/80, Cd14, or Cd68, and the inventors did see the expected obesity-related within-adipocyte changes of the adipocyte markers AdipoQ and Ccl2 (Table 6).

To examine whether these methylation changes between high-fat- and low-fat-fed mice involved changes in the expression of nearby genes, the inventors used quantitative PCR to examine the expression of 13 genes near genome-wide significant DMRs (FIG. 2B). The inventors used qPCR to examine mRNA from the same adipocytes and mice that were analyzed by CHARM. Of the 13 genes examined, 9 showed significant changes in mRNA expression in the opposite direction as methylation changes (FIG. 2B).

Furthermore, the inventors assessed whether these DNA methylation changes correlated with previously published genome-wide gene expression data in a similar cohort. The inventors saw significant inverse correlations between diet-related methylation changes and diet-related gene expression changes (FIGS. 8A and 8B). These results compare favorably to other functional analyses of discovered DMRs. Taken together, these data show that the inventors find robustly significant DMRs in mice that correlate with metabolic traits, that these DMRs replicate in separate animals, and that methylation at many of these regions appears to have a functional effect on gene expression.

FIGS. 8A-8B are graphical representations of data illustrating correlation of methylation and gene expression in mouse and human adipose tissue, related to FIG. 2. FIGS. 8A-8B show the relationship between methylation and gene expression in both mouse and human adipose tissues. Gene expression data was downloaded from GEO (see Materials and Methods) and plotted against mouse adipocyte and human adipose tissue CHARM data. Y-axes are the logarithm of the fold change (logFC) of the gene expression in high-fat-fed mice and obese humans versus low-fat-fed mice and lean humans. X-axes are the DNA methylation values calculated by CHARM (see Table Si of Feinberg et al. (Cell Metabolism 21(1):138-149 (2015))) for the high-fat versus low-fat mouse and obese versus lean human comparisons. Here, higher values indicate hypomethylation in high-fat/obese samples. P-values are for Pearson product-moment correlations versus a null hypothesis of no correlation.

Mouse DMRs Replicated Evolutionarily in Human Adipose Tissue

The inventors reasoned that many functionally relevant DMRs in mice exposed to a high-fat diet serve an important metabolic function that would be conserved across species and often susceptible to similar environmental cues. Therefore, to determine whether the methylation changes observed in mouse adipocytes could be replicated in an evolutionarily divergent cohort, the inventors performed CHARM analysis on human subcutaneous adipose tissues from 7 lean subjects and 14 obese, sex-matched, insulin-resistant subjects of the same age range, as well as 8 obese subjects post-RYGB.

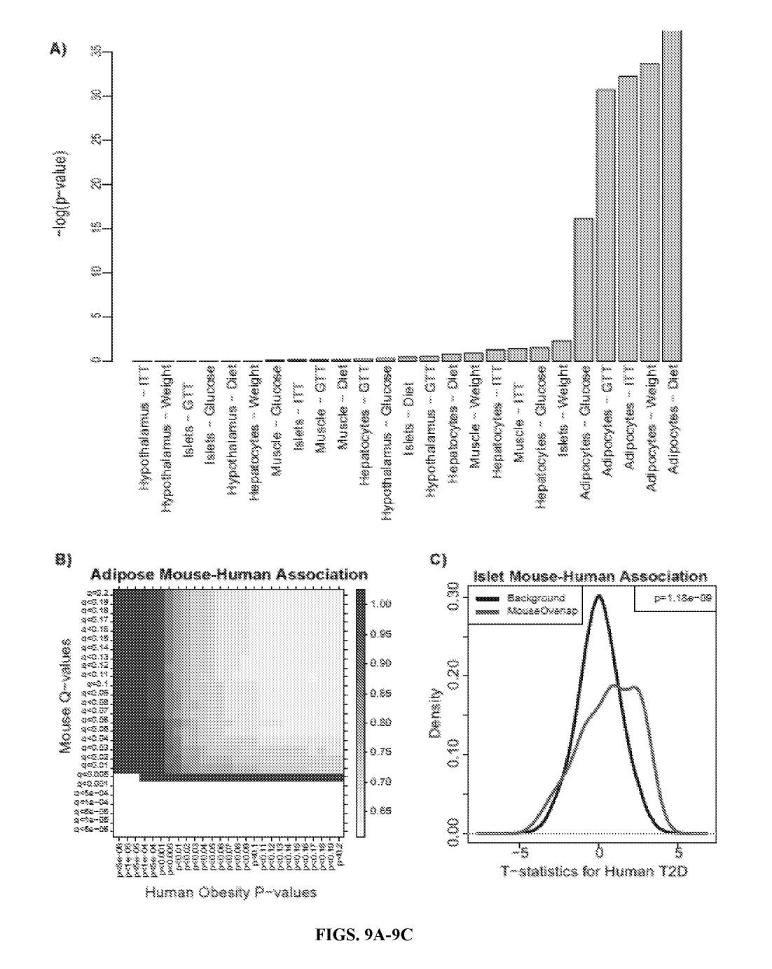

The inventors first examined the replication of mouse adipocyte DMRs in human adipose tissue from obese versus lean. The inventors observed very strong overlap between DMRs in human obese versus lean tissue and DMRs in high-fat-fed versus low-fat-fed mouse adipocytes (all p <10⁻¹⁵, FIG. 9A, rightmost five bars), showing that there is a strong correlation between areas that are regulated by methylation in metabolic dysfunction in both mice and humans.

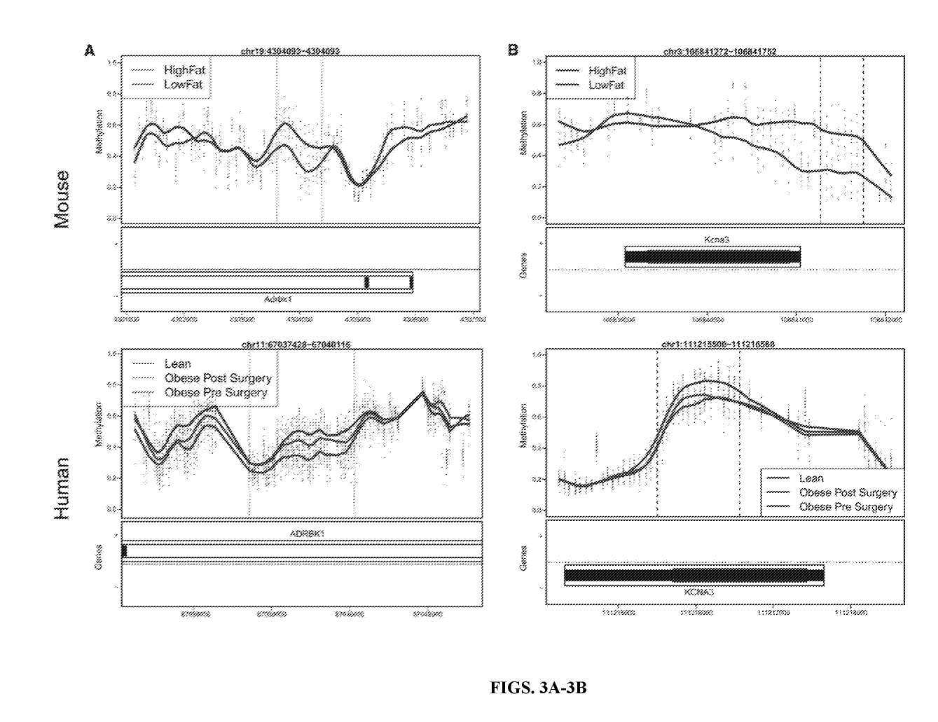

FIGS. 9A-9C are graphical representations of data illustrating significance of methylation change overlap between mouse and human tissues, related to FIG. 3.

In FIG. 9A, all 25 mouse analyses (x-axis) are compared against the human adipose obesity analysis. Values plotted represent the largest −log(p-value) for chi-squared tests for the overlap for all DMRs with nominal p-values <0.05 between the given mouse analysis and the human adipose obesity analysis. In FIG. 9B, for each square, the proportion of conserved mouse and human regions that had directionally consistent methylation changes in adipose tissue between species was calculated. Regions were required to have mouse and human methylation changes at or below the indicated Q-value for mouse and P-value for human. The color indicates the proportion of directionally consistent regions, with darker colors indicating a higher proportion. In FIG. 9C, the observed versus expected T-statistics for the proportion of overlap between the CHARM pancreatic islet mouse methylation data and the previously reported Illumina Infinium 450 k BeadChip™ pancreatic islet human methylation data.

Next, in order to determine which mouse methylation changes would replicate in human, the inventors determined that out of a total of 625 genome-wide significant mouse adipocyte DMRs, 576 had homologous regions on the human genome (hg19), calculated via the liftOver UCSC tool, and 497 had human CHARM probes within 5 kb. This is a remarkably high fraction (86.3%), suggesting that the assay method, CHARM, is highly comprehensive, and also that the location of CpG regions is strongly conserved in evolution. Of the 497 conserved DMRs, 249 (50.3%) showed significant differential methylation (p <0.05) between obese and lean people (Table 7). These numbers were similar when analyzing differential methylation before and after RYGB surgery (227 out of 497). As a final restrictive step in using human methylation to validate the mouse results, the inventors determined that 170 (68%) of these regions had a consistent direction of methylation change between high-fat-fed obese mice and obese humans, such that if a particular region had higher methylation in high-fat-fed mice, that region would also have higher methylation in obese humans and vice versa.

When more restrictive human methylation significance cutoffs are used, the percentage of regions with consistent directionality (true positive rate) rises, but the total number of retained regions drops, with 67/77 (87%) directionally consistent at human obesity p values <0.005, and 25/25 (100%) consistent at p values <0.0005 (FIG. 9B). All 170 directionally conserved regions were associated with the metabolic phenotypes of fasting glucose, GTT, and/or ITT in addition to mouse diet status. Furthermore, 134 of these regions had consistent directions of methylation change between both lean-obese and pre-/post-RYGB samples (e.g., higher in obesity and presurgery and vice versa), and a further 105 had postsurgery methylation values that were in between lean and presurgery methylation values, i.e., regions where methylation in obese subjects appeared to revert toward a lean phenotype after surgery (enrichment p=2.8×10-3).

In FIG. 3, the inventors present two regions that have significant methylation changes in human adipose tissue, are in homologous regions of the genome as mouse DMRs, are directionally consistent with the mouse DMRs, and have human postsurgery methylation levels that have moved closer to the lean phenotype. These regions are over two genes:ADRBK1 (adrenergic, beta, receptor kinase 1, FIG. 3A) and KCNA3 (potassium voltage-gated channel, shaker-related subfamily, member 3, FIG. 3B).

FIGS. 3A-3B are graphical representations of data illustrating overlapping methylation changes in human and mouse adipose tissue. For FIGS. 3A and 3B two genome-wide significant DMRs found in mouse adipocytes (top panels) over Adrbk1 (A) andKcna3 (B) are shown along with the corresponding methylation changes in human adipose tissue (bottom panels). For the panels denoting methylation, each point represents the methylation level from an individual mouse or human at a specific genomic location, with smoothed lines representing group methylation averages. y axis, methylation values. Below each methylation plot is a panel showing genomic coordinates for the respective species and any genes at those coordinates. See also FIG. 9 for tissue and species overlaps and Table 8 and Table 9 for conserved adipose mouse DMRs in human and for enrichment between DIAGRAM and conserved DMRs, respectively.

The inventors also assessed whether the human adipose DNA methylation changes correlated with previously published human genome-wide gene expression data from obese and lean individuals. As with the mouse data, the inventors saw a highly significant inverse correlation between obesity-related methylation changes and obesity-related gene expression changes (FIGS. 8A and 8B, right panels).

The inventors performed a similar mouse-human comparison in pancreatic islets using published DNAm data from T2D and control subjects, showing that 67% (odds ratio=7.2, p=7.2×10⁻⁶) of the mouse pancreatic islet DMRs that replicated in the human data had methylation change in the same direction and that these probes were far more associated with human T2D status than the rest of the probes on the array (p=1.18×10⁻⁹, FIG. 9C), demonstrating that the mouse-derived islet DMRs are enriched for potential epigenetic alteration in human T2D. Finally, the inventors also validated multiple mouse hepatocyte DMRs in human liver tissue, with 62.5% replicating (see Table S3 of Feinberg et al. (Cell Metabolism 21(1):138-149 (2015))).

Genetic Risk Loci Association with Overlapping Regions of Human and Mouse Methylation Changes

The inventors incorporated data from human GWAS for T2D using two complementary approaches that allow further characterization of the candidate obesity-related DMRs. GWAS summary statistics were obtained from the DIAGRAM (Diabetes Genetics Replication and Meta-Analysis) T2D genome-wide association meta-analysis, comprising data from 12 separate GWAS studies totaling 12,171 T2D cases and 56,682 controls (available on the World Wide Web at diagram-consortium.org). The inventors first directly explored the association between genes with obesity-related DMRs and genes conferring clinical genetic risk for T2D by calculating statistical enrichment of the GWAS regions overlapping the DMRs. The inventors found marginally significant enrichment for adipose DMRs among at least marginally significant GWAS signals (GWAS p value cutoffs starting with p <10-6, corresponding to enrichment p values ranging from 0.0048 to 0.0165, Table 8). Given the small number of directly overlapping regions, these results are likely strongly influenced by the strength of theTCF7L2 signal. While much of the early literature on TCF7L2 focused on its role in pancreatic islets, there is growing evidence that extrapancreatic effects may contribute to the T2D phenotype at this locus.

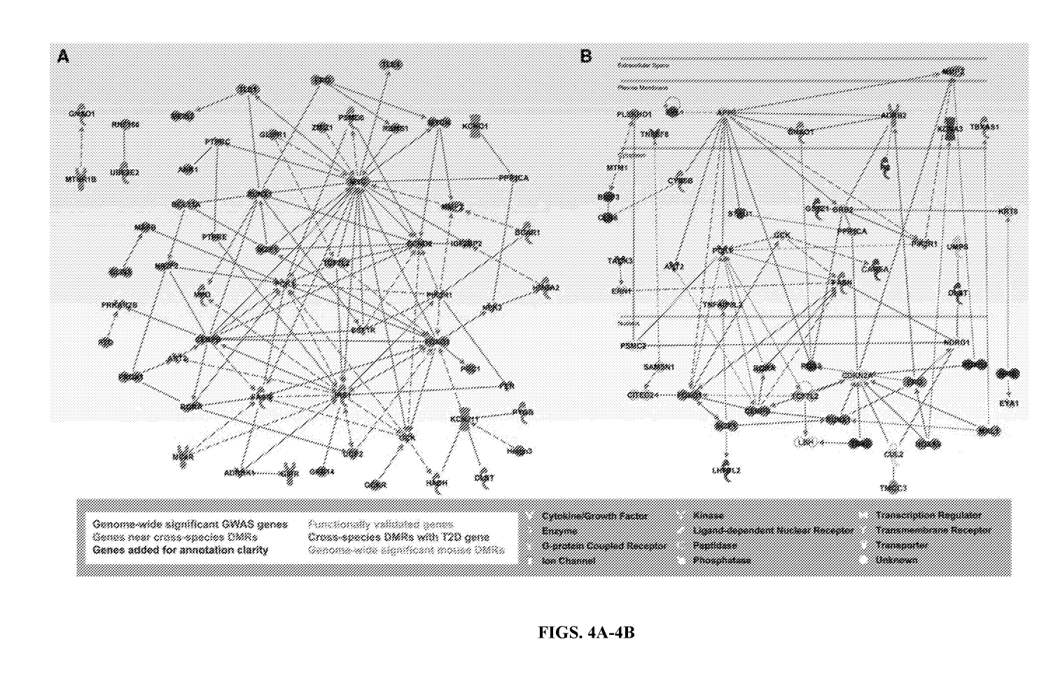

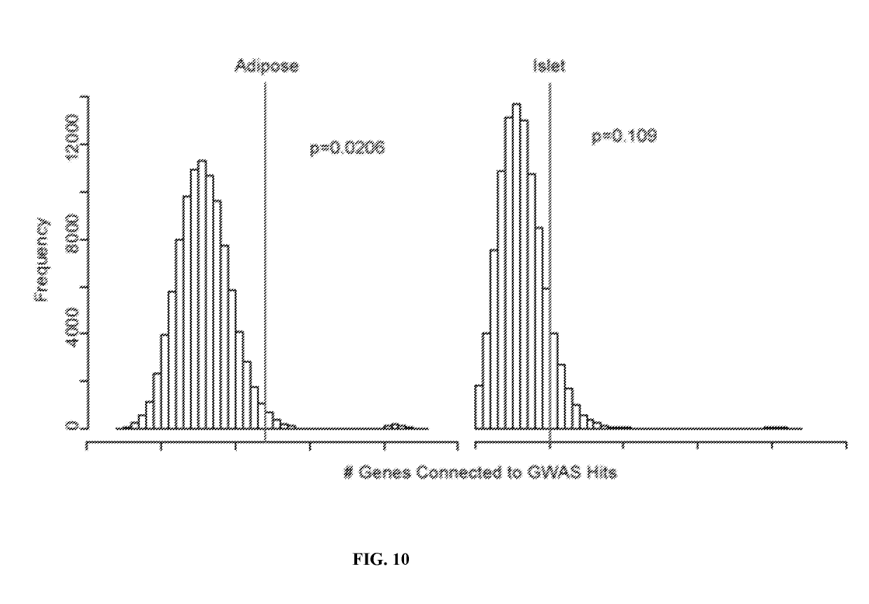

The inventors further examined statistical enrichment in the context of regulatory networks involving genes implicated in GWAS. Genes at 23 genome-wide significant GWAS signals (usually the gene nearest to the lead SNP) were directly (one-step) connected to genes near DMRs either by transcriptional control or direct protein-protein interaction (FIG. 4A). This amount of interaction represents significantly more than expected by random chance (p=0.0206) (FIG. 10) and demonstrates how genes implicated by methylation appear to be acting in the same pathways as genes implicated by GWAS. Similarly, expanding beyond one-step connections, many of the 30 regions implicated by both methylation data and GWAS are connected to genes identified by the mouse-only and human-mouse analyses and act in the same pathways (FIG. 4B).

FIGS. 4A-4B are diagrammatic representations of the interactions between epigenetically conserved and genetically associated genes implicated in this study. The data represented in the Figures was generated using QIAGEN's Ingenuity IPATM (Ingenuity Systems), and these diagrams represent the connections between genes implicated in the analyses. In FIG. 4A, genes with genome-wide significant linkage to T2D in the DIAGRAM meta-analysis were connected to genes near directionally conserved cross-species DMRs. Genes with no connections were dropped. In FIG. 4B, starting with a set of 23 genes near T2D-associated directionally conserved cross-species DMRs, this network was grown by adding genes near species-conserved and mouse-only genome-wide significant DMRs in order to represent one potential regulatory network. Gene colors explained in within-figure legend. See also FIG. 10 for the permutation analysis of the enrichment of interactions in FIG. 4A.

FIG. 10 is a graphical representation of data illustrating enrichment of connections between genes implicated by methylation and genome-wide significant GWAS genes, related to FIG. 4. This figure shows expected and observed connections and (both direct protein interactions and transcriptional control) and overlap between genes near species conserved adipose and islet DMRs and genes with genome-wide significant linkage to T2D in the DIAGRAM GWAS meta-analysis. The set of all possible one-step connections to the DIAGRAM GWAS genes was pulled from the Ingenuity Knowledge Base™, and the GWAS genes themselves were added. 100,000 permutations of random genes near DMRs were overlapped with this set, and the number of overlaps from the permutations are represented by the histograms. The actual number of observed DMRGWAS connections is denoted by the vertical red line, and the p-values represent permutation p-values for the difference between observed and expected connections.

Given these results, the inventors sought to further filter the obesity-related DMRs down to the subset of genes likely associated with T2D. The inventors hypothesize that DMRs that overlap associated marker SNPs for T2D can identify genes with epigenetic mechanisms of risk in adipose tissue. As many of the DMRs overlapping GWAS T2D loci with low p values implicate genes already known to be involved in T2D, obesity, and related phenotypes, the inventors therefore selected the subset of DMRs within genetic loci that had at least marginal statistical association with T2D clinical risk.

This approach reduced the 170 regions of directionally consistent and evolutionarily conserved methylation change in adipose tissue using the SNP-level summary statistics of the DIAGRAM analysis. In all, 30 cross-species and directionally conserved adipose DMRs directly overlapped with 27 marker SNPs (or close proxies with linkage disequilibrium >0.8) that had some evidence of association with T2D (at least p <0.01,Table 2; see Experimental Procedures). The inventors also identified ten regions where conserved pancreatic islet DMRs overlap with DIAGRAM SNPs (Table 9).

In these final 30 regions, not only have the inventors connected methylation change to obesity-induced metabolic phenotypes across two species, but the association with T2D-associated SNPs also provides a candidate mechanism for the methylation changes observed in human obesity and RYGB surgery. These 27 identified SNPs could potentially explain up to 2.69% of genetic T2D liability, though only one of these loci reached genome-wide significance in DIAGRAM. Even excluding this GWAS-positive loci (TCF7L2), which explains 1.12% of the variance alone, the remaining regions could explain up to 1.57% of genetic variance in T2D susceptibility. These data suggest that for at least some of these loci, genetic variation underlies changes in methylation that are causal for T2D risk. It is also possible that these regions are also susceptible to environmental factors that influence local methylation and that they therefore serve to integrate genetic and epigenetic effects.

Note that this filtering-based approach is independent of assessing the statistical enrichment of T2D GWAS signal, either at SNP or gene level, within the cross-species obesity-associated DMRs, an approach commonly used with GWAS summary statistic data. This approach therefore does not diminish the potential function of genes with GWAS-positive statistical association for T2D or of the DMRs that do not overlap with GWAS-associated SNPs, for contributing epigenetically to obesity.

The inventors hypothesized that one mechanism by which DNA methylation and genetic variation contribute to T2D risk may involve enhancer activity. Using publicly available human enhancer maps in 86 independent cell and tissue types, the inventors found that a striking proportion of DMRs mapped to adipose nuclei enhancers and superenhancers (which had the largest degree of overlap across all cell types). While the background proportion of overlap for CHARM was 17.2% for adipose enhancers and 3.8% for super enhancers, 40.6% (69 overlaps, p=1.58×10-15) and 14.7% (25 overlaps, p=5.72×10-13) of the directionally consistent 170 regions and 53.3% (16 overlaps, p=5.65×10-7) and 20% (6 overlaps, p=3.24×10-5) of the further 30 GWAS-associated regions above lie in adipose enhancers and super enhancers, respectively (Table 10). Thus, a major mechanism for methylation-mediated metabolic dysfunction is likely through epigenetic modification of enhancers. Note that most of these enhancers were not previously known to be related to T2D through conventional GWAS or other methods.

Functional Analysis of Genes Implicated by Cross-Species Methylation

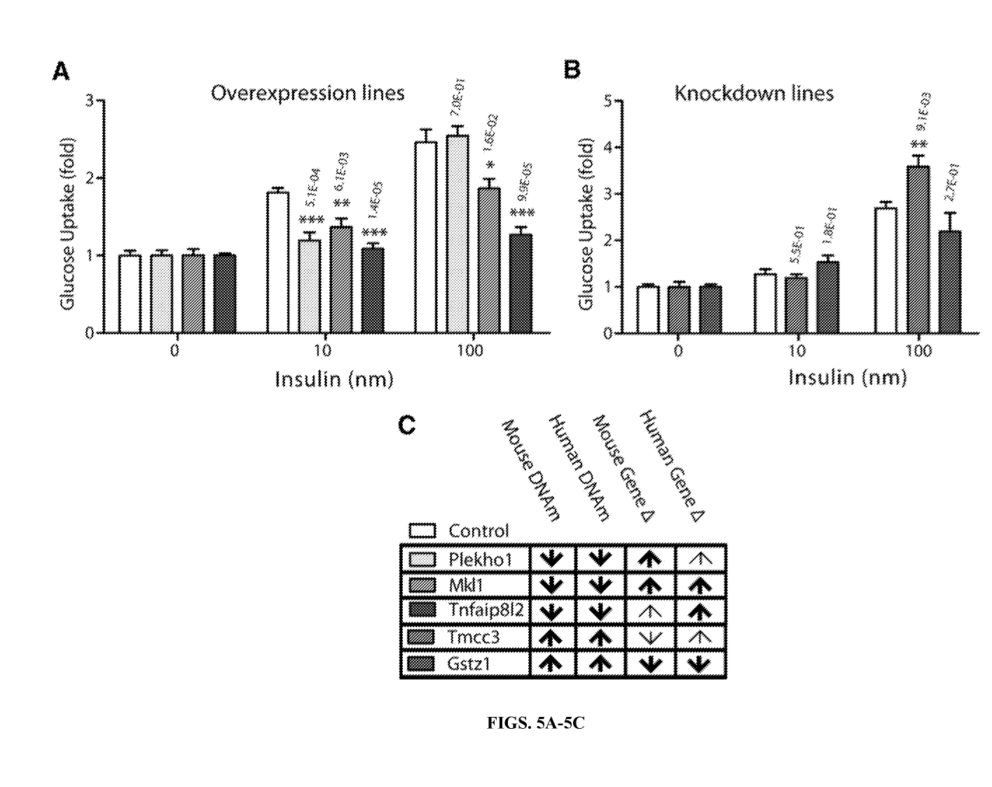

In order to establish that the cross-species method can identify functional genes implicated in obesity, insulin resistance, T2D, and related research, the inventors functionally assayed five genes. The inventors selected genes with no prior association with metabolic phenotypes and that had methylation reversion after RYGB. As RYGB is a targeted, environmental therapy that improves multiple deleterious phenotypes including insulin sensitivity, the inventors hypothesized that this subset of the results would be the most likely to have an effect on T2D- and obesity-related phenotypes. The inventors then examined the physiological effect of altering the expression of these genes on adipocyte cell culture models using insulin-stimulated glucose uptake assays. This procedure can measure the responsiveness of adipocytes to insulin, a phenotype disrupted in obesity. The inventors assayed seven 3T3-L1 adipocyte cell lines, each stably expressing shRNAs or expression plasmids corresponding to one of the five selected genes or a suitable control. In order to mimic the effects of a high-fat diet, genes hypermethylated in high-fat adipocytes were knocked down, and genes hypomethylated were overexpressed. Significant changes in glucose uptake were found for four of these five (FIG. 5B). Potential roles for all of these genes in modulating insulin sensitivity and resistance are considered in the Discussion below.

FIG. 5A-5C are graphical representation of data illustrating overexpression and shRNA-mediated knockdown of selected genes in 3T3-L1 adipocytes. For FIGS. 5A and 5B, selected genes from the set of 30 species conserved and T2D-SNP overlapping adipose DMRs were either stably overexpressed (A) or knocked down with shRNA (B). Glucose uptake is plotted as fold difference from normal, error bars represent standard error, and significance was determined by two-way ANOVA modified by Bonferroni correction denoted as follows: * p <0.05, **p <0.01, ***p <0.001. FIG. 5C shows DNA methylation and gene expression levels for high-fat-fed mice and obese human versus low-fat-fed mice and lean humans (e.g., “↓” indicates hypomethylation/lower gene expression in high-fat-fed and obese compared to low-fat-fed and lean). Bold arrows indicate significant changes.

Discussion

In mouse, the inventors identified 625 genome-wide significant DMRs that correlate with diet-induced obesity phenotypes in adipocytes. Of these regions, 249 had significant conserved methylation changes in human obesity, and 170 of these had the same direction of methylation change in both species. Thirty of these DMRs also overlapped with SNPs or nearby proxies that have been associated with human T2D genetic risk. These data show that DNA methylation changes in metabolic disease are conserved across species and that this conservation overlaps genomic regions where genetic polymorphisms have been associated with T2D. The approach combines three lines of evidence (epigenetic dysregulation following high-fat diet in mouse, epigenetic directional consistency in humans, and some evidence for clinical risk of T2D) to identify genes likely functionally implicated in the pathogenesis of T2D specifically through epigenetic mechanisms related to obesity.

In the present study, while the inventors use nominal p value significance to identify human methylation and GWAS results, the inventors first perform a multiple comparison correction in the initial set of mouse DMRs using a false discovery rate algorithm. As there is a growing awareness that the cumulative effect of common SNPs with low minor-allele frequency scores potentially explain large amounts of phenotypic variability beyond that of genome-wide significant SNPs identifiable by GWAS, approaches like ours that can use alternative methods to identify significant areas of potential genetic risk are necessary. The unique SNPs in these regions potentially account for 2.76% of T2D genetic variance, almost half of which is known by purely genetic analysis and may be epigenetically mediated.

The inventors observed significant changes associated with 4 out of 5 genes assayed by insulin-stimulated glucose uptake assay, a common indicator of insulin resistance. Screens using this assay and performed on sample sets not enriched for genes in gluco-insulinemic pathways have found a far smaller percentage of genes that will alter glucose uptake (˜10%), indicating that the method can successfully select potential targets with a much higher than random probability of affecting insulin sensitivity.

Three of the genes that the inventors found had altered glucose uptake fell into the classical inverse methylation-gene expression correlation: Mkl1, Plekho1, and Tnfaip812 were all hypomethylated in high-fat-fed mice and obese humans, had increased gene expression in corresponding subjects, and, when these genes were overexpressed in cell culture adipocytes, exhibited decreased glucose uptake in response to insulin, which would fit with the increased insulin resistance commonly observed in obesity and diabetes. While none of these genes has previously published roles in insulin resistance, several have suggestive links to metabolic phenotypes. Mkl1 is known to be a transcriptional coactivator of serum response factor (SRF), which been associated with insulin resistance in skeletal muscle. Similarly, PLEKHO1 has recently been shown to inhibit AKT/PI3K signaling, a pathway known to be involved in insulin signaling. With regards to the direction of glucose uptake change, the inventors note that insulin signaling induces both positive and negative feedback within affected cells, and without a methylation-gene expression candidate mechanism it is not possible to determine which feedback loop the methylation changes are involved with.

It is worth noting that as these genes did not contain common variants that passed the genome-wide significant GWAS threshold, they would not have been identified by GWAS alone. Similarly, only 4 out of these 5 genes had significant gene expression changes. This functional assay illustrates how the method of combining cross-species methylation data with GWAS results for common SNPs can implicate genes that would not have been detected otherwise.

Recent work in the laboratory has identified regions of the genome where DNA methylation acts to mediate a genetic effect on rheumatoid arthritis, and the methylation changes in obese humans could potentially act in an analogous role. The results in obese and insulin-resistant mouse models, however, identify methylation differences even between inbred mice and thus are definitively the result of environmental stimuli rather than a genetic underpinning. The fact that the inventors see many of these same methylation changes in obese humans, and that these changes are located over regions with known genetic links to T2D, implies that DNA methylation levels could be integrating and mediating genetic and environmental causes of metabolic disease at specific genomic loci.

It is encouraging that many of the genes described here show pathway relationships to known genetic associations (FIG. 4). For example, PRC1, a regulator of cytokinesis, is associated with T2D by a genome-wide significant DIAGRAM result, but it has no known connection to any other gene implicated by genome-wide significant DIAGRAM loci. Its transcription, however, is regulated by FOXO1, an important transcription factor in gluconeogenesis, insulin signaling, and adipocyte differentiation that the inventors find to be differentially methylated in both mouse and human obesity. FOXO1 is in turn regulated by TCF7L2, one of the strongest GWAS results. Furthermore, combining genes from all levels of this study creates potential regulatory networks that include genes with known involvement in T2D, but also incorporate closely connected genes with no previously known obesity or T2D association that are shown to be involved with obesity and insulin resistance in this story (FIG. 4B). Some of these genes, such as FASN and APP, appear to be loci in this network and could represent potentially important targets.

There are many approaches for and important applications of interrogating the association of functional and genetic elements using GWAS summary statistics (ENCODE Project Consortium, 2012), but the approach is unique in its leverage of carefully controlled biological systems to directly integrate cross-species functional epigenomics and clinical genetic risk by stratification. This work, of course, does not address or diminish the many GWAS associations that are not associated with methylation changes. Additionally, it is important to note that while the inventors do not directly address the issue of methylation causality in this study, causality is, at the least, multi-tiered. The functional data certainly indicate that these epigenetic changes are functionally proximate to T2D-relevant phenotypes and therefore important for discovery and for clinical translation. Current systems biology literature challenges conventional notions of causality as there is both positive and negative feedback in most complex living systems.

The approach described in this study may have broad applicability to identify candidate genes that may better dissect mechanisms and potential routes of treatment in common human disorders, such as cancer and cardiovascular disease. The accessibility of a limited cohort of relevant patients with well-characterized clinical materials before and after disease exposure is plausible for cross-species replication. This type of analysis can generate a reliable, functional candidate disease gene set that can be used to interrogate SNP data sets and lend additional support to specific targets that would not ordinarily pass the genome-wide correction threshold. The end result is a process that can integrate information from multiple complementary sources to identify potential targets essential for the pathogenesis of common diseases, such as obesity or T2D, that do not involve highly penetrant single genes, but rather arise from multiple defects along pathways that integrate genetic, epigenetic, and environmental cues.

Tables

TABLE 1
Genome-wide significant mouse DMRs.

| Tissue | Analysis | q val <0.05 | q val <0.1 |
|---|---|---|---|
| Adipocytes | Diet | 232 | 448 |
| | Weight | 183 | 288 |
| | Fasting glucose | 235 | 571 |
| | GTT | 0 | 3 |
| | ITT | 294 | 419 |

q values generated based upon comparison of observed DMR areas to areas generated by 1,000 random permutations of phenotype/methylation associations. See also Table S1 of Feinberg et al. (Cell Metabolism 21(1):138-149 (2015)) for a full list of all mouse DMRs.

TABLE 2
Mouse-human DMRs with genetic T2D risk loci association.

| | Relative location | Distance | RYGB | DIAGRAM p |
|---|---|---|---|---|
| Gene name | of DMR | to TSS | reversion | value |
| Tcf7l2 | inside intron | 43,058 | − | 4.90E−68 |
| Tcf7l2 | Inside intron | 77,345 | − | 4.90E−68 |
| As3mt | overlaps 5′ | 0 | + | 9.60E−06 |
| Etaa1 | Inside intron | 618 | + | 4.70E−05 |
| Tnfsf8 | overlaps 5′ | 0 | − | 0.00029 |
| Plekho1 | overlaps exon | 4,965 | + | 0.00045 |
| Tnfaip8l2 | inside intron | 337 | + | 0.00045 |
| Akt2 | inside intron | 20,427 | − | 0.00049 |

DIAGRAM GWAS 0.001 cutoff

| | | | | |
|---|---|---|---|---|
| Lhfpl2 | Inside intron | 2,490 | + | 0.001 |
| Mkl1 | overlaps 5′ | 0 | + | 0.0014 |
| BC048644 | overlaps exon | 146 | + | 0.0015 |
| (Car5a) | | | | |
| Rgs3 | downstream | 10,8842 | + | 0.0019 |
| Fgd3 | Inside intron | 11,100 | + | 0.002 |
| Stau1 | overlaps 5′ | 0 | + | 0.0022 |
| Tmcc3 | Inside intron | 43,772 | + | 0.0025 |
| Tbx3 | inside exon | 12,714 | − | 0.0029 |
| Gstz1 | Inside intron | 10,332 | + | 0.0029 |
| Taok3 | Inside intron | 549 | + | 0.0036 |
| Bnip3 | Inside intron | 1,863 | − | 0.0039 |
| Dlst | overlaps 5′ | 0 | + | 0.0053 |
| Kcna3 | Close to 3′ | 2,192 | + | 0.0064 |
| Cln8 | Inside intron | 3,055 | + | 0.0065 |
| Cd37 | exon | 2,687 | + | 0.0069 |
| Nfib | Inside intron | 100,380 | − | 0.0071 |
| Pck1 | promoter | 453 | + | 0.0072 |
| Pck1 | overlaps 5′ | 0 | + | 0.0072 |
| Pcx | inside intron | 59,049 | + | 0.0073 |
| Hoxd3 | inside intron | 7,307 | + | 0.0084 |
| Cd33 | overlaps 5′ | 0 | + | 0.0087 |
| Evl | exon | 157 | + | 0.0099 |

Shown are the names of the nearest gene to the mouse and human differential methylation, the position of the DMR relative to the gene, the distance to the transcriptional start site (TSS), whether the direction of methylation change (sign of smoothed effect statistic) post-RYGB surgery reverts toward lean subject methylation levels (RYGB reversion), and the p value of the T2D genetic association in the region. See also Table 9 for an analogous table with the pancreatic islet results instead and Table 10 for conserved adipose DMRs that overlap with adipose enhancers.

TABLE 3
Pyrosequencing primers.

| | | SEQ. |
|---|---|---|
| Pyrosquencing | | ID. |
| Primers | Sequence | NO. |
| Runx1 Long 5′ | TTGAGTTTGTTAAATTTAGGGGTAAGT | 1 |
| Runx1 Long 3′ | TTCAAACAACATTTTTAAATCATTTC | 2 |
| Runx1 Nested 5′ | TTGAGTTTGTTAAATTTAGGGGTAAGT | 3 |
| Runx1 Nested 3′ | /5Biosg/CAAACAAAAACTATTAACTCTAAAACCAC | 4 |
| Runx1 Sequencing 1 | GTTATTAGAGTAAGTGTA | 5 |
| Runx1 Sequencing 2 | GATAAAATTTAAAGAGTGTT | 6 |
| Runx1 Sequencing 3 | GTTTAAGTATTTATTAAAATAT | 7 |
| Pscdbp Long 5′ | TTTTTTAGGGAAAAGAATTTTTTTT | 8 |
| Pscdbp Long 3′ | ATCTCAAACCACAACAATCACATAA | 9 |
| Pscdbp Nested 5′ | GGTATTATTTTTAGTAGAGGTTAGGTAAA | 10 |
| Pscdbp Nested 3′ | /5Biosg/CAAAACCAAAAAACCATATATTAAC | 11 |
| Pscdbp Sequencing 1 | GGTATTATTTTTAGTAGAGGTTAGGTA | 12 |
| Pscdbp Sequencing 2 | ATAGTGTTTGTTAGTATTTGAAT | 13 |
| Stap1 Long 5′ | TTTTATAATAATTGAAGGAGGGAAAGT | 14 |
| Stap1 Long 3′ | AAAAACAATATAACCCAAACAAAAAC | 15 |
| Stap1 Nested 5′ | TTTTATAATAATTGAAGGAGGGAAAGT | 16 |
| Stap1 Nested 3′ | /5Biosg/TACACACTTACCTAATAATCAAACC | 17 |
| Stap1 Sequencing 1 | GAATAGTTGTTTTTTTTTTTAATAT | 18 |
| Stap1 Sequencing 2 | AATTTTAAAGTAAAGGTTATG | 19 |
| Pck1 Long 5′ | TGGTAAAGGTTTTGTTGTTTAAGTGT | 20 |
| Pck1 Long 3′ | ATTCTCTAACCATCCCAAAATAAAC | 21 |
| Pck1 Nested 5′ | TTTTTAGATATTTGGGTATTTAAGA | 22 |
| Pck1 Nested 3′ | /5Biosg/ACTATAAACTTTATTCTAACAAAACAATAC | 23 |
| Pck1 Sequencing 2 | GTTTGATGTATATTTTTTTG | 24 |
| Pck1 Sequencing 3 | TTTAGAGTAGGGGTTAGTAT | 25 |
| Dguok Long 5′ | AGTTGTATAATTTATTGTGGGTTGG | 26 |
| Dguok Long 3′ | TCCTTTAAAACTTCCTCAAACATTT | 27 |
| Dguok Nested 5′ | AGTTATGGAGGGTTAATTTGTGTTT | 28 |
| Dguok Nested 3′ | /5Biosg/TCCTTTAAAACTTCCTCAAACATTT | 29 |
| Dguok Sequencing 1 | GTTAATTTGTGTTTGGGATT | 30 |
| Dguok Sequencing 2 | ATTTTTAGAGGATTGGGTGAAG | 31 |
| Dguok Sequencing 3 | GGAGTTATTGATAAATTT | 32 |
| Kcnj11 Long 5′ | AGAGTTTAGGTTATAGGTGGGAGGT | 33 |
| Kcnj11 Long 3′ | AAAATATCCTACCAACCAAAAAAAA | 34 |
| Kcnj11 Nested 5′ | TTTAGGTTATGTTTAAGGGTTTTGG | 35 |
| Kcnj11 Nested 3′ | /5Biosg/AAAACTAAAAAACCCACACAAACAC | 36 |
| Kcnj11 Sequencing 1 | ATTTTTTTTTTTAGTAATTTAGATAAG | 37 |
| Kcnj11 Sequencing 2 | ATTTTATTTTTTAGTTTTTGG | 38 |
| Kcnj11 Sequencing 3 | GAGGTGAGTTTAGGTAGATTT | 39 |
| Zfhnx2 Long 5′ | TTTTGAGGGTTTAGTGGTAGTTTGT | 40 |
| Zfhnx2 Long 3′ | CAAATTCAATAAAAACACAAATTAAAAAA | 41 |
| Zfhnx2 Nested 5′ | AGGGTTTAGTGGTAGTTTGTTAGGTTAT | 42 |
| Zfhnx2 Nested 3′ | /5Biosg/AAAAAAAATCAACAAAAACAATATTAAATT | 43 |
| Zfhnx2 Sequencing 1 | AAATTGTTTATTTTTTGTAG | 44 |
| Zfhnx2 Sequencing 2 | TTTTTTATTTTATTTTATTTTTT | 45 |
| Slc38a4 Long 5′ | GGAGTTTTTATTAGGAGAGGGTGTAG | 46 |
| Slc38a4 Long 3′ | ATAAACCAAAATAACCTCCAACTCA | 47 |
| Slc38a4 Nested 5′ | TATTTTGATGGTGTTAGAGATGAATTT | 48 |
| Slc38a4 Nested 3′ | /5Biosg/CCACCACCACCTAAAAAAAACT | 49 |
| Slc38a4 Sequencing 1 | ATGGTGTTAGAGATGAATT | 50 |
| Slc38a4 Sequencing 2 | GTATAGTTATATTTTTAAAT | 51 |
| Slc38a4 Sequencing 3 | TTATTTGATGTTTAATATATTT | 52 |
| Chpt1 Outer F | GGGAGTTTAGAATAGTGATTGGTTG | 53 |
| Chpt1 Outer R | TCCCTTCCTTAAATAACCTTCCTAC | 54 |
| Chpt1 Inner F | GGGAAGATTTTTAAATAGAGAGGATGT | 55 |
| Chpt1 Outer R | /5BiosG/TACCCTAAAAACTAAACCCCAAAAC | 56 |
| Chpt1 Seq2 | GATATTTAATATAATATTTATTT | 57 |
| Chpt1 Seq3 | ATTGAGTTTTAGGTTAGTTTTA | 58 |
| Chpt1 Seq4 | TTGGGGGAATTAATTTAAGT | 59 |
| Liph Outer F | ATAGTTTAGGAGGAAAGTTGGTGTT | 60 |
| Liph Outer R | AAAAACTTCCAAAACATAAAAAAAA | 61 |
| Liph Inner F | AAGGGATTTAAGGGATTTTTAAATTT | 62 |
| Liph Inner R | /5BiosG/CAACTCCCTACAACAAAACACTTT | 63 |
| Liph Seq1 | GGTAGTTTGGTTTATTT | 64 |
| Liph Seq2 | GATTATTTAGATGTTTGT | 65 |
| Liph Seq3 | GTGGTATGATAGGTAGTAG | 66 |
| Fasn Outer F | GGGTTTTAAGAGGTTGTTGGTTAAT | 67 |
| Fasn Outer R | AACCCTAAACAAAACACAATTTCAC | 68 |
| Fasn Inner F | TAGTTTGTTTTGGGATAGGTTGTG | 69 |
| Fasn Inner R | /5BiosG/AATATACCCCCAAAAATAAAAAACC | 70 |
| Fasn Seq1 | GAATATAAAGGTTAAGTGTTTA | 71 |
| Fasn Seq2 | AGAGTTTGGGTAGTTAGATAG | 72 |
| Fasn Seq3 | AGGGTTTTTATTTTTATTAAG | 73 |
| Fasn Seq4 | GTTAATTAAATTTTTTAATTTG | 74 |
| Axin2 Outer F | GATTTTTAAGGAGGGGATTTTGTAG | 75 |
| Axin2 Outer R | CTATCCCACATCACCAATCTAAACT | 76 |
| Axin2 Inner F | TTTTTTTATTTTATGTGGTGTTTGT | 77 |
| Axin2 Inner R | /5BiosG/ACTAAAACTATCCCTACCTATTCCTC | 78 |
| Axin2 Seq1 | TTTTATTTTATGTGGTGTT | 79 |
| Axin2 Seq2 | TAATTGTTTTTGTTTTTTG | 80 |
| Axin2 Seq3 | GAATTTTAGAGTGAGGATTTG | 81 |
| Scd1 Outer F | AGAAGGTTTGGGGTAATATAGAAGTTT | 82 |
| Scd1 Outer R | AAATCCCCTCTCCTTAAAACATAAC | 83 |
| Scd1 Inner F | TGGGAAATTTTTTGATAGTT | 84 |
| Scd1 Inner R | /5Biosg/AATTCTACTAAATCCTCAAAAAAACTAAAC | 85 |
| Scd1 Seq1 | TTTGGGTTATATATGTGTTA | 86 |
| Scd1 Seq2 | GTTTTTGTATTTGTGAGGG | 87 |
| Scd1 Outer F | AAGGGAGGTTTTTGTTATTTATTTA | 88 |
| Scd1 Outer R | ACCTTCCTTATAACCATCAATTCC | 89 |
| Scd1 Inner F | TGTTTTTTAGTAAGTGAGAAGAGATGGT | 90 |
| Scd1 Inner R | /5Biosg/AACCAAATTTAAACCCAACCTAAAC | 91 |
| Scd1 Seq1 | GTGGTTTAGAAAGAAGAGTTTTGT | 92 |
| Fermt2 Outer F | TGTGGTAAGGTTTATTTTTTAGAGG | 93 |
| Fermt2 Outer R | AAACAAACTTTTATCTCCCCTTTTAC | 94 |
| Fermt2 Inner F | ATAATAGAGGATAGAAATAAAAGAATGAAA | 95 |
| Fermt2 Inner R | /5Biosg/CCTTCAAATTATATAAATTTCAAATAATAA | 96 |
| Fermt2 Seq1 | ATTTTAGAAGTTATAATTTAAGTA | 97 |
| Atp6v0a1 Outer F | TTTTGGAATTAGTTTAAAAGGGTTG | 98 |
| Atp6v0a1 Outer R | AACAAAAAACAAAACAAAACCAAAT | 99 |
| Atp6v0a1 Inner F | ATTTTTAAGTAGGGATTTTTTTGTGAG | 100 |
| Atp6v0a1 Inner R | /5Biosg/TCATCCTAATACCTTCAAACTACTCTC | 101 |
| Atp6v0a1 Seq1 | GATAAGATTTTAATGTATTTAAGTT | 102 |
| Arhgap29 Outer F | TATGGATTTTGGGATTTTTGATTAT | 103 |
| Arhgap29 Outer R | TACAACAACCTAACCAACAAAAAAA | 104 |
| Arhgap29 Inner F | TTTTAATTTTGAATTTAGAGGAAATTTAGT | 105 |
| Arhgap29 Inner R | /5Biosg/AAAATATTTAATAAATTTCTATTCCCCC | 106 |
| Arhgap29 Seq1 | GTTATTTGTATTTTTGTTAAATT | 107 |
| Arhgap29 Seq2 | TTGTAAAGTGTTTGTTGATAA | 108 |
| Arhgap29 Seq3 | ATTAGATATTTTTGTTATAATTT | 109 |
| Masp1 Outer F | TATGTGTATTTATATTTGGGATTTTTTTAG | 110 |
| Masp1 Outer R | AATAAAACTCTTCTAACCCCTAAACTC | 111 |
| Masp1 Inner F | GAAGTTTGTGTTGTTTGTATTTTTG | 112 |
| Masp1 Inner R | /5Biosg/TCTATATTTACTTAAAACATACCCTC | 113 |
| Masp1 Seq1 | GGTTTTGGTGTTTTTGGAGTGGGAGA | 114 |
| Masp1 Seq2 | GTTATGTAGTAGGTGAAATGAGTT | 115 |
| Elovl5 Outer F | TTGTTGTATAGGTTTGTAGTTTAGGAGTAA | 116 |
| Elovl5 Outer R | TCAAAAACCCAATTAAATCAATATTC | 117 |
| Elovl5 Inner F | AAAATTGTTTAAGAGTATATTTTTTAAAAA | 118 |
| Elovl5 Inner R | /5Biosg/AACATCCAACATTAATTTCCTTACC | 119 |
| Elovl5 Seq1 | ATTTTTATTGTTAAGTTATATATGATT | 120 |
| Elovl5 Seq2 | TAGAGGTTTTTTAAAGAATGTG | 121 |
| Elovl5 Seq3 | ATTTTGTAAATTAAATTAGATGG | 122 |
| Tmem140 Outer F | TATAGAATGATGTTTATAAAGTGGGATATA | 123 |
| Tmem140 Outer R | AATTAAAAACCCCATAACACTCTTCT | 124 |
| Tmem140 Inner F | GGGATATAAATATATATATTTATGTAATTT | 125 |
| Tmem140 Inner R | /5Biosg/AACCAATATTTCCTTCAAAAAACAA | 126 |
| Tmem140 Seq1 | AGAAGTGTGTTGTTTAGAGTGGT | 127 |

TABLE 4
Quantitative PCR primers.

| Quantitative | | SEQ. |
|---|---|---|
| PCR | | ID. |
| Primers | Sequence | NO. |
| Tnks1bp1 F | CCCAGGACCCTCACTCCAT | 128 |
| Tnks1bp1 R | TCCCAAACTCCCAGTCTTGAA | 129 |
| Fbxw8 F | GCCAGGTTGCCTTTGGAGT | 130 |
| Fbxw8 R | TCCCGGATGTTGACACAGGTA | 131 |
| Sorbs1 F | CCCCGTCTGAGGTAATAGTTGT | 132 |
| Sorbs1 R | GAGCAGTCTCCAGGAGTATAGTC | 133 |
| Vps13c F | GAAGCTAAAGTAAAAGCCCACGA | 134 |
| Vps13c R | ACACATCAGAGGTGTTGACAATG | 135 |
| Tcf712 F | AACGAACACAGCGAATGTTTCC | 136 |
| Tcf712 R | CACCTTGTATGTAGCGAACGC | 137 |
| Pcx F | CTGAAGTTCCAAACAGTTCGAGG | 138 |
| Pcx R | CGCACGAAACACTCGGATG | 139 |
| Tnfsf8 F | GCACAAGTCGCAGCTACTTCT | 140 |
| Tnfsf8 R | GGAGTGGAGTCCTTTTTCTGG | 141 |
| Etaa1 F | GGTGGCACGGGAATGAGTC | 142 |
| Etaa1 R | GATTTGTACTGGCGTCTCCTTT | 143 |
| Pck1 F | CTGCATAACGGTCTGGACTTC | 144 |
| Pck1 R | CAGCAACTGCCCGTACTCC | 145 |
| Rgs3 F | GCTTCCTGTAGGACAAGACCT | 146 |
| Rgs3 R | GGCTTTGAGGGGGCTTAGG | 147 |
| Stau1 F | GGACCCTCACTCTCGGATG | 148 |
| Stau1 R | TTCTGGCAGGGGTTCACTCT | 149 |
| As3mt F | GGGAATGTACTGAAGACATCTGC | 150 |
| As3mt R | CCACAGCCATAATACCTCGAACT | 151 |
| Akt2 F | ACGTGGTGAATACATCAAGACC | 152 |
| Akt2 R | GCTACAGAGAAATTGTTCAGGGG | 153 |
| Tnfaip812 F | TCAGCTCAAAGAGTCTGGCAC | 154 |
| Tnfaip812 R | GGTAAAGCTCGTCTAGCACCTC | 155 |

TABLE 5
Gene ontology for genes near DMRs, related to Table 1.

| | Gene Ontology Biological | | | Enrichment Q- |
|---|---|---|---|---|
| DMR set | Process Term | Count | List Total | value |
| Adipocyte | triglyceride metabolic | 9 | 47 | 3.33E−04 |
| Hypermethylated DMRs | process | | | |
| | acylglycerol metabolic | 9 | 56 | 8.23E−04 |
| | process | | | |
| | neutral lipid metabolic | 9 | 58 | 7.51E−04 |
| | process | | | |
| | positive regulation of | 4 | 6 | 2.15E−03 |
| | cholesterol storage | | | |
| | low-density lipoprotein | 4 | 7 | 3.96E−03 |
| | particle clearance | | | |
| | cellular carbohydrate | 11 | 131 | 9.12E−03 |
| | metabolic process | | | |
| Adipocyte | regulation of response to | 81 | 2173 | 3.13E−05 |
| Hypomethylated DMRs | stimulus | | | |
| | neutrophil chemotaxis | 8 | 32 | 7.50E−04 |
| | positive regulation of | 48 | 1110 | 5.10E−04 |
| | response to stimulus | | | |
| | cell activation | 23 | 334 | 4.70E−04 |
| | myeloid leukocyte activation | 10 | 59 | 4.13E−04 |
| | immune system process | 38 | 784 | 3.70E−04 |

Genes near genome-wide significant DMRs (q-value <0.05) for adipocyte-fasting glucose associations were submitted to the Gene Ontology enRIchment anaLysis and visuaLizAtion tool (GOrilla) along with a background of all the genes possible to find on the applicable array. The list of genes found in adipocytes was first divided into hypomethylated and hypermethylated groups depending on the status of the corresponding DMR. Here, hypermethylation refers to areas where increased methylation is associated with higher fasting glucose and hypomethylation the converse.

TABLE 6
Results of qPCR assay to test adipose tissue purification,
related to FIG. 2.

| Gene | Fold change (high fat/low fat) | SD | P-value |
|---|---|---|---|
| Adipoq | −1.41 | 0.4 | 0.039 |
| Pparg | −0.58 | 0.3 | 0.167 |
| Ccl2 | 1.29 | 0.32 | 0.043 |
| Albumin | −0.08 | 1.32 | 0.476 |
| CD68 | 1.69 | 0.9 | 0.174 |
| Corola | 0.02 | 1.41 | 0.494 |
| F4/80 | 0.06 | 0.64 | 0.463 |
| CD14 | 0.27 | 0.38 | 0.341 |

This table shows the results of the quantitative PCR assay to test if the mouse adipocyte tissue samples were pure.

TABLE 7
Conserved mouse-human DMRs, related to FIG. 3.

| | | | | | | Nearest | Human | Obesity | Human | RYGB |
|---|---|---|---|---|---|---|---|---|---|---|
| | | | | | Nearest | Gene | Obesity | Methylation | RYGB | Methylation |
| Chr | Start | End | Wid | qval | Gene | Distance | p. val | Slope | p. val | Slope |
| chr1 | 6804060 | 6804159 | 100 | 0.044 | Dnajc11 | 31055 | 0.05396806 | −0.127370809 | 0.076926702 | −0.096956819 |
| chr1 | 19804495 | 19805076 | 582 | 0.005 | Capzb | 6360 | 0.009584786 | −0.102400227 | 0.080255224 | −0.065423927 |
| chr1 | 23171771 | 23172254 | 484 | 0.006 | Ephb2 | 123359 | 0.163730493 | −0.037281993 | 0.240294807 | −0.014835724 |
| chr1 | 27681877 | 27682185 | 309 | 0.012 | Map3k6 | 11634 | 0.017716616 | −0.077604526 | 0.009129994 | 0.064042072 |
| chr1 | 36191885 | 36192039 | 155 | 0.044 | Clspn | 44625 | 0.239591101 | −0.036206241 | 0.201558347 | 0.045218473 |
| chr1 | 37838402 | 37839079 | 678 | 0.032 | Zc3h12a | 79935 | 0.378536274 | −0.015552746 | 0.001183568 | 0.17839242 |
| chr1 | 48351513 | 48351750 | 238 | 0.011 | OTTMU | 93473 | 0.000271516 | 0.138474484 | 0.105292916 | 0.064390092 |
| | | | | | SG00000008561 | | | | | |
| chr1 | 61909979 | 61910398 | 420 | 0.026 | Nfia | 321628 | 0.283317249 | 0.065328087 | 0.171060478 | 0.054337879 |
| chr1 | 63253431 | 63254413 | 983 | 0.006 | Atg4c | 5876 | 0.090799505 | 0.029828429 | 0.02784476 | −0.076102611 |
| chr1 | 64061530 | 64062519 | 990 | 0.006 | Pgm2 | 1768 | 0.023274675 | 0.09603559 | 0.2239538 | −0.057666211 |
| chr1 | 65733701 | 65734201 | 501 | 0.012 | Dnajc6 | 39493 | 0.297952055 | −0.036724144 | 0.02627919 | −0.087078963 |
| chr1 | 94153416 | 94153898 | 483 | 0.009 | Bcar3 | 5588 | 0.641591964 | 0.012994303 | 0.358888654 | 0.024734722 |
| chr1 | 1E+08 | 1E+08 | 515 | 0.005 | Palmd | 901 | 0.191059976 | 0.020861026 | 0.035225878 | −0.087686438 |
| chr1 | 1E+08 | 1E+08 | 77 | 0.042 | Slc35a3 | 565 | 0.045055579 | 0.053255223 | 0.373986866 | 0.029616686 |
| chr1 | 1.01E+08 | 1.01E+08 | 465 | 0.005 | Dbt | 1015 | 0.026114662 | −0.043754768 | 0.266052273 | −0.016109601 |
| chr1 | 1.11E+08 | 1.11E+08 | 489 | 0.009 | Kcna3 | 2193 | 0.000830083 | −0.174664378 | 0.026296074 | −0.050827227 |
| chr1 | 1.11E+08 | 1.11E+08 | 449 | 0.006 | Kcna3 | 677 | 0.028609698 | 0.071486755 | 0.078482013 | −0.10916874 |
| chr1 | 1.14E+08 | 1.14E+08 | 941 | 0.006 | Ptpn22 | 288 | 8.28E−05 | −0.140138463 | 0.111418668 | −0.051964801 |
| chr1 | 1.14E+08 | 1.14E+08 | 609 | 0.005 | Ptpn22 | 0 | 8.28E−05 | −0.140138463 | 0.111418668 | −0.051964801 |
| chr1 | 1.14E+08 | 1.14E+08 | 354 | 0.006 | Ptpn22 | 972 | 8.28E−05 | −0.140138463 | 0.111418668 | −0.051964801 |
| chr1 | 1.5E+08 | 1.5E+08 | 244 | 0.022 | Otud7b | 424 | 0.481806589 | 0.013695731 | 0.038840057 | −0.037841131 |
| chr1 | 1.5E+08 | 1.5E+08 | 305 | 0.012 | Plekho1 | 4964 | 0.011515715 | −0.061527856 | 0.092524395 | −0.035311704 |
| chr1 | 1.51E+08 | 1.51E+08 | 425 | 0.005 | Tnfaip812 | 336 | 2.58E−05 | −0.203542029 | 0.043102817 | −0.102319904 |
| chr1 | 1.54E+08 | 1.54E+08 | 206 | 0.026 | S100a4 | 632 | 0.015459655 | 0.111432355 | 0.006174879 | 0.124908817 |
| chr1 | 1.55E+08 | 1.55E+08 | 562 | 0.044 | Flad1 | 888 | 0.063435358 | −0.088485589 | 0.062015242 | 0.026512865 |
| chr1 | 1.61E+08 | 1.61E+08 | 692 | 0.005 | Cd48 | 0 | 0.037990596 | −0.040921991 | 0.058606544 | −0.048173527 |
| chr1 | 1.61E+08 | 1.61E+08 | 392 | 0.005 | Arhgap30 | 636 | 4.80E−09 | −0.264158562 | 0.010337311 | −0.114725719 |
| chr1 | 1.61E+08 | 1.61E+08 | 389 | 0.006 | Fcgr3 | 411 | 0.002652877 | −0.16864877 | 0.017223425 | −0.125052231 |
| chr1 | 1.73E+08 | 1.73E+08 | 1177 | 0.005 | Tnfsf4 | 359 | 0.082438786 | −0.056016855 | 0.477320041 | −0.015276966 |
| chr1 | 1.73E+08 | 1.73E+08 | 233 | 0.017 | Prdx6 | 3000 | 0.102115617 | 0.04274556 | 0.003004404 | 0.096005652 |
| chr1 | 1.82E+08 | 1.82E+08 | 403 | 0.017 | Glul | 2739 | 0.068397296 | 0.062977644 | 0.025488547 | −0.098159346 |
| chr1 | 1.93E+08 | 1.93E+08 | 438 | 0.006 | Rgs1 | 0 | 0.015727132 | −0.133234131 | 0.016067624 | −0.102588829 |
| chr1 | 1.99E+08 | 1.99E+08 | 2388 | 0.005 | Ptprc | 0 | 0.018214222 | −0.100339811 | 0.001093455 | −0.147785599 |
| chr1 | 1.99E+08 | 1.99E+08 | 460 | 0.045 | Ptprc | 39084 | 0.407309588 | −0.037295152 | 0.278995332 | −0.0199901 |
| chr1 | 1.99E+08 | 1.99E+08 | 719 | 0.005 | Ptprc | 368025 | 0.176595525 | −0.042360171 | 0.012370438 | 0.11232229 |
| chr1 | 2E+08 | 2E+08 | 220 | 0.022 | Nr5a2 | 258072 | 0.163636918 | −0.04654987 | 0.249703563 | −0.022766232 |
| chr1 | 2.12E+08 | 2.12E+08 | 731 | 0.012 | Nek2 | 658 | 0.01220632 | 0.100825287 | 0.061637418 | 0.091412309 |
| chr1 | 2.12E+08 | 2.12E+08 | 1610 | 0.005 | Ints7 | 45038 | 0.205896102 | 0.032334064 | 0.032263572 | −0.111604731 |
| chr1 | 2.21E+08 | 2.21E+08 | 224 | 0.026 | Mark1 | 51166 | 0.173358467 | 0.062705846 | 0.198390864 | 0.054456176 |
| chr1 | 2.21E+08 | 2.21E+08 | 511 | 0.005 | C130074 | 995 | 0.191583115 | −0.055482328 | 0.216499795 | −0.044018923 |
| | | | | | G19Rik | | | | | |
| chr1 | 2.22E+08 | 2.22E+08 | 569 | 0.005 | Dusp10 | 5715 | 0.107649879 | −0.033404002 | 0.038084354 | −0.038682285 |
| chr1 | 2.3E+08 | 2.3E+08 | 415 | 0.026 | Galnt2 | 33499 | 0.067540877 | −0.041459111 | 0.021183552 | −0.085751104 |
| chr1 | 2.36E+08 | 2.36E+08 | 143 | 0.044 | Nid1 | 69287 | 0.428590605 | −0.012324001 | 0.360883807 | −0.013890278 |
| chr1 | 2.42E+08 | 2.42E+08 | 271 | 0.012 | Opn3 | 2726 | 0.004056696 | −0.109795008 | 0.002465152 | 0.141015585 |
| chr1 | 2.48E+08 | 2.48E+08 | 257 | 0.043 | Trim58 | 941 | 0.200965059 | −0.040951299 | 0.004083849 | 0.148912371 |
| chr2 | 10093863 | 10094145 | 283 | 0.036 | Grhl1 | 6541 | 0.149489146 | −0.059018061 | 0.042991352 | −0.098039251 |
| chr2 | 16084776 | 16085714 | 939 | 0.024 | Mycn | 3829 | 0.026462974 | −0.037242163 | 0.020034154 | −0.042121351 |
| chr2 | 21151188 | 21151541 | 354 | 0.006 | Apob | 91845 | 0.006293461 | −0.043007646 | 0.079009519 | −0.025968826 |
| chr2 | 25668139 | 25668364 | 226 | 0.049 | Dtnb | 166912 | 0.117496348 | −0.099659161 | 0.041252876 | −0.126276622 |
| chr2 | 38669779 | 38670055 | 277 | 0.015 | Arl6ip2 | 52758 | 0.192013286 | 0.04128462 | 0.164498053 | 0.036971722 |
| chr2 | 44308569 | 44309760 | 1192 | 0.005 | Lrpprc | 82575 | 0.183046934 | 0.088509005 | 0.36710835 | 0.074797085 |
| chr2 | 45911195 | 45911602 | 408 | 0.035 | Prkce | 33264 | 0.277667197 | −0.043119313 | 0.34129699 | −0.035189023 |
| chr2 | 46795289 | 46796098 | 810 | 0.009 | Rhoq | 23492 | 0.022247323 | −0.09536594 | 0.343882477 | 0.042790041 |
| chr2 | 47216781 | 47216960 | 180 | 0.024 | Ttc7 | 34850 | 0.001719588 | 0.178933354 | 0.177314794 | 0.068088499 |
| chr2 | 62426656 | 62427018 | 363 | 0.012 | B3gnt2 | 2506 | 0.021726827 | −0.05800806 | 0.007112103 | 0.124450687 |
| chr2 | 63270337 | 63271211 | 875 | 0.006 | Ehbp1 | 276814 | 0.105911246 | 0.032484673 | 0.092082131 | 0.025752407 |
| chr2 | 67625367 | 67625994 | 628 | 0.011 | Etaa1 | 587 | 0.014192426 | −0.159468397 | 0.057427316 | −0.081615983 |
| chr2 | 68592241 | 68593052 | 812 | 0.005 | Plek | 0 | 0.000417575 | −0.116575262 | 0.001208315 | −0.14693488 |
| chr2 | 68961351 | 68961704 | 354 | 0.012 | Arhgap25 | 233 | 0.000287308 | −0.169520485 | 0.009437236 | −0.102669374 |
| chr2 | 68962521 | 68963097 | 577 | 0.005 | Arhgap25 | 532 | 0.000287308 | −0.169520485 | 0.009437236 | −0.102669374 |
| chr2 | 70211010 | 70211141 | 132 | 0.006 | Asprv1 | 17464 | 0.074793806 | −0.040295727 | 0.008663913 | 0.040009929 |
| chr2 | 80531572 | 80531693 | 122 | 0.044 | Lrrtm1 | 64 | 0.01267564 | −0.12761776 | 0.001646793 | 0.138411511 |
| chr2 | 1.01E+08 | 1.01E+08 | 287 | 0.03 | Lonrf2 | 156474 | 0.037745155 | −0.103856175 | 0.308482567 | −0.050985187 |
| chr2 | 1.2E+08 | 1.2E+08 | 286 | 0.012 | Dbi | 4474 | 0.096817539 | 0.042198961 | 0.122807867 | −0.044198351 |
| chr2 | 1.2E+08 | 1.2E+08 | 257 | 0.03 | Sctr | 1272 | 0.049402728 | −0.060074519 | 0.037452731 | 0.081224769 |
| chr2 | 1.37E+08 | 1.37E+08 | 284 | 0.029 | Cxcr4 | 5558 | 0.025691591 | −0.101355347 | 0.286970636 | −0.041871989 |
| chr2 | 1.45E+08 | 1.45E+08 | 299 | 0.011 | Zeb2 | 122956 | 0.013432772 | −0.148539776 | 0.433483904 | 0.03426897 |
| chr2 | 1.58E+08 | 1.58E+08 | 1374 | 0.005 | Pscdbp | 313 | 0.003845787 | −0.126579923 | 0.009591686 | −0.113021718 |
| chr2 | 1.58E+08 | 1.58E+08 | 841 | 0.005 | Pscdbp | 350 | 0.003845787 | −0.126579923 | 0.009591686 | −0.113021718 |
| chr2 | 1.59E+08 | 1.59E+08 | 768 | 0.005 | Acvr1 | 29817 | 0.345419057 | −0.037080746 | 0.380691236 | −0.029122073 |
| chr2 | 1.77E+08 | 1.77E+08 | 325 | 0.033 | Hoxd3 | 22043 | 0.00782395 | −0.119911701 | 0.009779601 | −0.072776117 |
| chr2 | 2.34E+08 | 2.34E+08 | 685 | 0.005 | Atgl6l1 | 551 | 0.408179201 | 0.018866827 | 0.108141227 | −0.041195482 |
| chr2 | 2.37E+08 | 2.37E+08 | 372 | 0.008 | Gbx2 | 1887 | 0.014302383 | −0.228144487 | 0.001711165 | −0.057789286 |
| chr2 | 2.42E+08 | 2.42E+08 | 227 | 0.006 | Kif1a | 30209 | 0.157313624 | 0.04528784 | 0.130219629 | 0.032731766 |
| chr6 | 1608907 | 1609124 | 218 | 0.033 | Foxc1 | 903 | 0.02149946 | −0.144758449 | 0.026549578 | −0.067922771 |
| chr6 | 6701849 | 6702647 | 799 | 0.009 | Ly86 | 113412 | 0.138073502 | −0.044960274 | 0.016446104 | −0.102655949 |
| chr6 | 8069397 | 8069892 | 496 | 0.029 | Eef1e1 | 17563 | 0.230098757 | 0.03717546 | 0.529955234 | −0.010570813 |
| chr6 | 10557112 | 10557387 | 276 | 0.015 | Gcnt2 | 657 | 0.084712725 | 0.085695391 | 0.051183188 | 0.099709759 |
| chr6 | 20149392 | 20149620 | 229 | 0.042 | Mboat1 | 61723 | 0.357755126 | −0.029006955 | 0.111157742 | −0.033338785 |
| chr6 | 26595986 | 26596063 | 78 | 0.044 | Abt1 | 849 | 0.001774016 | 0.091203913 | 0.029453493 | 0.047168264 |
| chr6 | 43488870 | 43489065 | 196 | 0.03 | Rpo1-1 | 3770 | 0.039655641 | −0.063665591 | 0.002944446 | −0.080512729 |
| chr6 | 43977929 | 43978138 | 210 | 0.026 | Mrpl14 | 112658 | 0.067770138 | −0.049733177 | 0.106801536 | −0.048325515 |
| chr6 | 72001391 | 72001673 | 283 | 0.015 | Ogfrl1 | 2607 | 0.650063432 | 0.021514144 | 0.394440329 | 0.045118385 |
| chr6 | 75913118 | 75913414 | 297 | 0.011 | Col12a1 | 2313 | 0.039964536 | 0.036258368 | 0.018634341 | 0.043387645 |
| chr6 | 79922240 | 79922533 | 294 | 0.009 | Hmgn3 | 27731 | 0.031223875 | 0.101608036 | 0.133122901 | −0.046937456 |
| chr6 | 83774408 | 83774634 | 227 | 0.03 | Ube2cbp | 549 | 0.088148366 | 0.024298645 | 0.111115821 | −0.083926315 |
| chr6 | 90995934 | 90996135 | 202 | 0.026 | Bach2 | 10138 | 0.31033621 | −0.018624689 | 0.050662463 | 0.062412637 |
| chr6 | 1.07E+08 | 1.07E+08 | 222 | 0.02 | Atg5 | 98735 | 0.032929012 | −0.092864789 | 0.005575201 | −0.1356305 |
| chr6 | 1.08E+08 | 1.08E+08 | 261 | 0.011 | Scml4 | 26 | 0.0002544 | −0.15054077 | 0.0011841 | −0.119484281 |
| chr6 | 1.1E+08 | 1.1E+08 | 632 | 0.006 | 5033413 | 26320 | 0.07455092 | −0.113810962 | 0.089160776 | −0.100483874 |
| | | | | | D22Rik | | | | | |
| chr6 | 1.22E+08 | 1.22E+08 | 146 | 0.044 | D630037 | 0 | 0.214211871 | −0.076381082 | 0.065115908 | 0.035362635 |
| | | | | | F22Rik | | | | | |
| chr6 | 1.23E+08 | 1.23E+08 | 315 | 0.006 | Hsf2 | 708 | 0.782800905 | −0.005988991 | 0.090043395 | −0.090159621 |
| chr6 | 1.36E+08 | 1.36E+08 | 265 | 0.026 | Ahi1 | 69750 | 0.150413734 | −0.027839173 | 0.071477084 | 0.028903577 |
| chr6 | 1.36E+08 | 1.36E+08 | 283 | 0.026 | Pde7b | 192747 | 0.138900626 | 0.033768848 | 0.031368491 | 0.061275681 |
| chr6 | 1.4E+08 | 1.4E+08 | 525 | 0.005 | Cited2 | 2998 | 0.071358568 | −0.05626553 | 0.0250784 | −0.088202576 |
| chr6 | 1.43E+08 | 1.43E+08 | 221 | 0.028 | Gpr126 | 69582 | 0.060847098 | 0.063670558 | 0.005923013 | 0.077463306 |
| chr6 | 1.55E+08 | 1.55E+08 | 303 | 0.032 | A130090 | 72682 | 0.054281324 | −0.064447241 | 0.016348248 | −0.072805909 |
| | | | | | K04Rik | | | | | |
| chr6 | 1.64E+08 | 1.64E+08 | 727 | 0.005 | Qk | 17101 | 0.005485027 | 0.144576733 | 0.039999443 | −0.095803967 |
| chr6 | 1.65E+08 | 1.65E+08 | 479 | 0.005 | Qk | 569295 | 0.06111785 | −0.049408781 | 0.072372323 | −0.043504723 |
| chr8 | 1715132 | 1715419 | 288 | 0.012 | Cln8 | 3056 | 0.020312359 | −0.055086638 | 0.1977797 | −0.031975276 |
| chr8 | 17529635 | 17529755 | 121 | 0.047 | Mtus1 | 28971 | 0.212448998 | −0.045221446 | 0.081591051 | −0.056975457 |
| chr8 | 23184678 | 23184867 | 190 | 0.044 | Loxl2 | 59455 | 0.033107474 | −0.028156664 | 0.086812392 | −0.024788173 |
| chr8 | 26429564 | 26429693 | 130 | 0.047 | EG432870 | 3988 | 0.089238216 | −0.060735815 | 0.073336461 | 0.048605512 |
| chr8 | 28226653 | 28226969 | 317 | 0.005 | Zfp395 | 16863 | 0.070140437 | −0.097469136 | 0.149103273 | −0.08306212 |
| chr8 | 29191531 | 29191754 | 224 | 0.044 | Dusp4 | 13370 | 0.074664184 | 0.033243325 | 0.011987359 | −0.148375941 |
| chr8 | 30402021 | 30402307 | 287 | 0.011 | Rbpms | 123000 | 0.059656602 | 0.066092099 | 0.074840217 | 0.051525926 |
| chr8 | 37384529 | 37384565 | 37 | 0.015 | Zfp703 | 163716 | 0.056355149 | −0.074172948 | 0.025017061 | 0.054509768 |
| chr8 | 41907137 | 41907600 | 464 | 0.006 | Myst3 | 1715 | 0.033951722 | −0.049325479 | 0.032881859 | 0.092351017 |
| chr8 | 43152678 | 43152944 | 267 | 0.012 | 4921537 | 2731 | 0.101774677 | 0.10468965 | 0.001638054 | 0.071895792 |
| | | | | | P18Rik | | | | | |
| chr8 | 48921440 | 48921670 | 231 | 0.017 | Ube2v2 | 343 | 0.549728625 | 0.045360426 | 0.723948231 | −0.032717176 |
| chr8 | 66701734 | 66702089 | 356 | 0.006 | Pde7a | 415 | 0.000239452 | 0.133829622 | 0.022705523 | 0.060627845 |
| chr8 | 70588403 | 70588535 | 133 | 0.047 | Slco5a1 | 115217 | 0.03737239 | −0.118897956 | 0.027050638 | −0.095556995 |
| chr8 | 72271032 | 72271813 | 782 | 0.005 | Eya1 | 2632 | 0.006526032 | 0.055557647 | 0.039520429 | 0.068107877 |
| chr8 | 79577640 | 79577971 | 332 | 0.012 | 3110050 | 302 | 0.027335434 | −0.120166491 | 0.013070318 | −0.060396267 |
| | | | | | N22Rik | | | | | |
| chr8 | 86377077 | 86377304 | 228 | 0.012 | Car2 | 950 | 0.205873288 | 0.079580679 | 0.186796984 | 0.039905492 |
| chr8 | 1.1E+08 | 1.1E+08 | 213 | 0.032 | Nudcd1 | 0 | 0.005606233 | −0.146137424 | 0.051823134 | −0.087134642 |
| chr8 | 1.24E+08 | 1.24E+08 | 1123 | 0.005 | Derl1 | 2649 | 0.016160863 | −0.201700733 | 0.089516329 | 0.026571651 |
| chr8 | 1.26E+08 | 1.26E+08 | 254 | 0.017 | Mtss1 | 82626 | 0.053762046 | 0.044152252 | 0.016721463 | −0.073319669 |
| chr8 | 1.26E+08 | 1.26E+08 | 670 | 0.005 | Mtss1 | 63675 | 0.105146153 | 0.033045224 | 0.109370839 | 0.020581098 |
| chr8 | 1.26E+08 | 1.26E+08 | 409 | 0.012 | Sqle | 227 | 0.018217776 | 0.088449027 | 0.056776096 | 0.09498824 |
| chr8 | 1.29E+08 | 1.29E+08 | 500 | 0.026 | Myc | 152321 | 0.029892253 | 0.074853732 | 0.013289519 | 0.075490732 |
| chr8 | 1.31E+08 | 1.31E+08 | 904 | 0.03 | 0910001 | 58431 | 0.006432972 | 0.083997065 | 0.053704732 | −0.0768861 |
| | | | | | A06Rik | | | | | |
| chr8 | 1.31E+08 | 1.31E+08 | 377 | 0.006 | Ddef1 | 919 | 0.091831138 | −0.05001441 | 0.025151831 | −0.103694855 |
| chr8 | 1.41E+08 | 1.41E+08 | 553 | 0.009 | 18100 | 189344 | 0.457540782 | 0.018224753 | 0.107424574 | −0.04111049 |
| | | | | | 44A24Rik | | | | | |
| chr8 | 1.45E+08 | 1.45E+08 | 378 | 0.012 | AA409316 | 4016 | 0.026970252 | −0.06895942 | 0.004605833 | 0.117723719 |
| chr10 | 1402817 | 1403101 | 285 | 0.033 | Adarb2 | 371069 | 0.008030471 | −0.051764156 | 0.093009663 | −0.035032577 |
| chr10 | 3895156 | 3895674 | 519 | 0.026 | Klf6 | 58172 | 0.055394379 | 0.036857931 | 0.25164663 | −0.019903847 |
| chr10 | 5485881 | 5486200 | 320 | 0.012 | Net1 | 22431 | 0.081087469 | −0.097455401 | 0.118015948 | −0.068368104 |
| chr10 | 5490224 | 5490845 | 622 | 0.005 | Net1 | 1444 | 0.149235639 | 0.058475426 | 0.216396182 | 0.038191461 |
| chr10 | 5708419 | 5708659 | 241 | 0.012 | Asb13 | 0 | 0.059598542 | −0.04917547 | 0.007263468 | 0.084979203 |
| chr10 | 6190543 | 6191242 | 700 | 0.006 | Rbm17 | 54400 | 0.017146089 | −0.073584795 | 0.019469104 | −0.07219703 |
| chr10 | 21571119 | 21571557 | 439 | 0.005 | Nebl | 121815 | 0.30595094 | −0.036460642 | 0.299758058 | −0.021884691 |
| chr10 | 25305780 | 25305987 | 208 | 0.05 | Thnsl1 | 179 | 0.101278552 | −0.053808868 | 0.028015814 | −0.078205159 |
| chr10 | 70884710 | 70884845 | 136 | 0.05 | Vps26a | 671 | 0.087081301 | −0.069417137 | 0.034050172 | 0.062072851 |
| chr10 | 73300885 | 73301322 | 438 | 0.005 | Cdh23 | 129697 | 0.080482241 | 0.055621735 | 0.089036951 | −0.031747595 |
| chr10 | 82175262 | 82175938 | 677 | 0.009 | 5730469 | 7325 | 0.130456278 | 0.059308364 | 0.137804668 | 0.067323607 |
| | | | | | M10Rik | | | | | |
| chr10 | 82180528 | 82180732 | 205 | 0.015 | 5730469 | 9852 | 0.061117646 | 0.080669928 | 0.120311064 | −0.081479412 |
| | | | | | M10Rik | | | | | |
| chr10 | 82279570 | 82279941 | 372 | 0.028 | Tspan14 | 60467 | 0.013820019 | −0.139269935 | 0.00467758 | 0.075130378 |
| chr10 | 91006627 | 91007328 | 702 | 0.008 | Lipa | 2702 | 0.074112974 | −0.077302683 | 0.116122616 | 0.040575054 |
| chr10 | 97144444 | 97145224 | 781 | 0.005 | Sorbs1 | 48677 | 0.027219413 | 0.07734614 | 0.001956324 | −0.071898348 |
| chr10 | 97517128 | 97517433 | 306 | 0.032 | Entpd1 | 1149 | 0.026474464 | −0.077219508 | 0.061995517 | −0.046133335 |
| chr10 | 98416374 | 98417030 | 657 | 0.006 | Pik3ap1 | 52458 | 0.002273819 | −0.160411722 | 0.116710163 | −0.070339204 |
| chr10 | 99160683 | 99160877 | 195 | 0.044 | Rrp12 | 186 | 0.080496726 | −0.057407697 | 0.116057055 | 0.075788125 |
| chr10 | 1.02E+08 | 1.02E+08 | 210 | 0.022 | Scd1 | 1993 | 0.049406421 | −0.040765323 | 0.189306401 | −0.060816137 |
| chr10 | 1.03E+08 | 1.03E+08 | 418 | 0.033 | Btrc | 112265 | 0.057694353 | −0.085267828 | 0.012106663 | −0.164397338 |
| chr10 | 1.05E+08 | 1.05E+08 | 762 | 0.005 | As3mt | 1892 | 0.004186243 | 0.118357444 | 0.018854098 | 0.05787133 |
| chr10 | 1.05E+08 | 1.05E+08 | 294 | 0.026 | Sh3pxd2a | 157260 | 0.000643737 | 0.11508831 | 0.000122591 | 0.095521028 |
| chr10 | 1.13E+08 | 1.13E+08 | 140 | 0.044 | Adra2a | 2305 | 0.153991099 | −0.061788635 | 0.056353675 | 0.126197788 |
| chr10 | 1.15E+08 | 1.15E+08 | 448 | 0.035 | Tcf7l2 | 42599 | 0.00035593 | 0.158699963 | 0.353841481 | −0.038008249 |
| chr10 | 1.15E+08 | 1.15E+08 | 830 | 0.009 | Tcf7l2 | 76886 | 0.001798348 | 0.127830599 | 0.008485517 | −0.065095072 |
| chr10 | 1.15E+08 | 1.15E+08 | 479 | 0.006 | Tcf7l2 | 109944 | 0.100312432 | 0.057000362 | 0.060303239 | 0.086105446 |
| chr10 | 1.16E+08 | 1.16E+08 | 806 | 0.009 | Adrb1 | 21804 | 0.272925161 | −0.0481567 | 0.14171411 | −0.042851261 |
| chr10 | 1.18E+08 | 1.18E+08 | 295 | 0.006 | Atrnl1 | 404090 | 0.162784849 | 0.055245791 | 0.385223126 | −0.03548502 |
| chr10 | 1.23E+08 | 1.23E+08 | 343 | 0.017 | Brwd2 | 0 | 0.157144273 | −0.045241863 | 0.032353194 | 0.096461872 |
| chr10 | 1.24E+08 | 1.24E+08 | 144 | 0.03 | Tacc2 | 112343 | 0.042701586 | −0.050719844 | 0.010493753 | −0.044891116 |
| chr10 | 1.24E+08 | 1.24E+08 | 305 | 0.012 | Plekha1 | 877 | 0.198129182 | 0.050715394 | 0.101876959 | −0.053856557 |
| chr10 | 1.29E+08 | 1.29E+08 | 217 | 0.022 | Dock1 | 980 | 0.055414321 | −0.036175689 | 0.217399231 | 0.044818476 |
| chr10 | 1.3E+08 | 1.3E+08 | 447 | 0.005 | Ptpre | 114341 | 0.006558867 | −0.157671746 | 0.022388868 | −0.093969223 |
| chr10 | 1.32E+08 | 1.32E+08 | 487 | 0.006 | Glrx3 | 155537 | 0.259498246 | 0.021822295 | 0.048281286 | 0.042428248 |
| chr10 | 1.33E+08 | 1.33E+08 | 595 | 0.015 | Tcerg1l | 182449 | 0.023710989 | 0.071039385 | 0.016816568 | 0.07245519 |
| chr10 | 1.34E+08 | 1.34E+08 | 162 | 0.046 | Bnip3 | 1862 | 0.032971233 | 0.105305104 | 0.022670161 | −0.09346149 |
| chr10 | 1.35E+08 | 1.35E+08 | 235 | 0.006 | 6430531 | 4090 | 0.04716606 | −0.118765257 | 0.02780257 | 0.053334838 |
| | | | | | B16Rik | | | | | |
| chr10 | 1.35E+08 | 1.35E+08 | 1195 | 0.006 | Cyp2e1 | 8867 | 0.098711704 | 0.049678185 | 0.037460112 | 0.078945329 |
| chr12 | 1663136 | 1663829 | 694 | 0.005 | Wnt5b | 21848 | 0.043716238 | −0.046192522 | 0.049488495 | 0.070552105 |
| chr12 | 6166239 | 6166263 | 25 | 0.011 | Vwf | 50822 | 0.248521563 | 0.043982022 | 0.022024994 | −0.079555472 |
| chr12 | 6469500 | 6469539 | 40 | 0.006 | Scnn1a | 11615 | 0.03543076 | 0.03100671 | 0.071225305 | 0.042570688 |
| chr12 | 7060921 | 7061200 | 280 | 0.032 | Ptpn6 | 424 | 0.000293744 | −0.145500528 | 0.01348777 | −0.08116907 |
| chr12 | 7308164 | 7308415 | 252 | 0.015 | Clstn3 | 30684 | 0.035942937 | −0.126870767 | 0.024351012 | 0.062460805 |
| chr12 | 8216915 | 8217820 | 906 | 0.006 | C3ar1 | 1261 | 0.114586135 | −0.033344046 | 0.056823806 | −0.080856054 |
| chr12 | 8275965 | 8276317 | 353 | 0.026 | Clec4a4 | 138 | 0.013803864 | −0.124748032 | 0.010435524 | −0.083632699 |
| chr12 | 11812172 | 11812279 | 108 | 0.022 | Etv6 | 9226 | 0.005150697 | 0.120887626 | 0.023111836 | 0.055501587 |
| chr12 | 14928365 | 14928608 | 244 | 0.016 | H2afj | 872 | 0.115301893 | −0.022948594 | 0.007076155 | −0.116071913 |
| chr12 | 14995468 | 14996149 | 682 | 0.006 | Art4 | 197 | 0.047220256 | 0.076344607 | 0.103695806 | −0.069175548 |
| chr12 | 15111717 | 15112453 | 737 | 0.005 | Arhgdib | 1519 | 0.00956376 | −0.100766318 | 0.029894071 | −0.074887817 |
| chr12 | 26392741 | 26393036 | 296 | 0.031 | Sspn | 34525 | 0.032865677 | 0.114081438 | 0.136647608 | −0.074148874 |
| chr12 | 40010029 | 40010986 | 958 | 0.005 | Abcd2 | 2759 | 0.045439035 | −0.052089161 | 0.014124607 | −0.077337776 |
| chr12 | 50419685 | 50419898 | 214 | 0.017 | Racgap1 | 450 | 0.067364544 | −0.028632215 | 0.055732133 | 0.056997761 |
| chr12 | 53268780 | 53268925 | 146 | 0.047 | Krt8 | 26221 | 0.417767017 | −0.0228861 | 0.558644222 | 0.044011508 |
| chr12 | 53497808 | 53497996 | 189 | 0.044 | Soat2 | 448 | 0.059851859 | 0.04586622 | 0.01856073 | −0.041060574 |
| chr12 | 54686256 | 54686746 | 491 | 0.04 | Nfe2 | 6386 | 0.047686589 | −0.052394071 | 0.137424206 | −0.03739656 |
| chr12 | 62658334 | 62658491 | 158 | 0.047 | Usp15 | 3106 | 0.012820313 | −0.080748795 | 0.01870305 | −0.101326683 |
| chr12 | 69140644 | 69140871 | 228 | 0.022 | Slc35e3 | 687 | 0.00294898 | −0.05840808 | 0.174567673 | 0.028840302 |
| chr12 | 75874872 | 75875171 | 300 | 0.005 | Glipr1 | 375 | 0.028932969 | −0.079091903 | 0.133593051 | −0.061851978 |
| chr12 | 76662008 | 76662319 | 312 | 0.033 | Osbpl8 | 174550 | 0.230407077 | 0.084261368 | 0.366828779 | 0.047464744 |
| chr12 | 94978299 | 94978583 | 285 | 0.03 | Tmcc3 | 61933 | 0.117548072 | −0.021978496 | 0.007529745 | −0.075171522 |
| chr12 | 95000081 | 95001986 | 1906 | 0.026 | Tmcc3 | 43751 | 0.004387317 | 0.116714272 | 0.167215876 | 0.026028237 |
| chr12 | 96813903 | 96814310 | 408 | 0.006 | Pctk2 | 16844 | 0.097873514 | 0.084093689 | 0.077434262 | 0.102265772 |
| chr12 | 1.01E+08 | 1.01E+08 | 36 | 0.017 | Uhrf1bp1l | 3883 | 0.2054361 | 0.045371194 | 0.01143517 | 0.100915673 |
| chr12 | 1.02E+08 | 1.02E+08 | 285 | 0.011 | Slc5a8 | 59109 | 0.068941654 | −0.057969332 | 0.047063901 | 0.048213168 |
| chr12 | 1.02E+08 | 1.02E+08 | 1912 | 0.005 | Chpt1 | 1466 | 0.086619668 | 0.103113517 | 0.219034114 | 0.08831166 |
| chr12 | 1.03E+08 | 1.03E+08 | 629 | 0.005 | Pmch | 33525 | 0.004350063 | 0.115790802 | 0.264567514 | 0.049532817 |
| chr12 | 1.04E+08 | 1.04E+08 | 221 | 0.017 | Tdg | 752 | 0.005057558 | −0.136091463 | 0.056719502 | −0.035545438 |
| chr12 | 1.07E+08 | 1.07E+08 | 127 | 0.026 | Ckap4 | 16968 | 0.036423307 | 0.046232273 | 0.03125838 | −0.116741557 |
| chr12 | 1.07E+08 | 1.07E+08 | 236 | 0.022 | Rfx4 | 55034 | 0.452216789 | 0.039977643 | 0.661016157 | 0.018901322 |
| chr12 | 1.09E+08 | 1.09E+08 | 164 | 0.049 | Ssh1 | 41530 | 0.017916085 | 0.035229392 | 0.057734338 | −0.057221546 |
| chr12 | 1.1E+08 | 1.1E+08 | 453 | 0.005 | Kctd10 | 56723 | 0.495308113 | 0.036933183 | 0.107609453 | 0.050279414 |
| chr12 | 1.1E+08 | 1.1E+08 | 318 | 0.012 | Git2 | 478 | 0.240157029 | 0.050905326 | 0.030801005 | 0.073031388 |
| chr12 | 1.15E+08 | 1.15E+08 | 291 | 0.033 | Tbx3 | 12908 | 0.039598932 | −0.062105131 | 0.080103137 | 0.058777531 |
| chr12 | 1.15E+08 | 1.15E+08 | 197 | 0.017 | Tbx3 | 3086 | 0.00275724 | −0.14756063 | 0.001537324 | −0.068605604 |
| chr12 | 1.15E+08 | 1.15E+08 | 867 | 0.005 | Tbx3 | 6515 | 0.00030568 | −0.187331894 | 0.001286375 | −0.141162287 |
| chr12 | 1.17E+08 | 1.17E+08 | 21 | 0.05 | 2410131 | 1337 | 0.095202773 | −0.072915207 | 0.428718333 | 0.022901692 |
| | | | | | K14Rik | | | | | |
| chr12 | 1.17E+08 | 1.17E+08 | 1006 | 0.006 | Fbxw8 | 65405 | 0.047336201 | −0.0429966 | 0.007012339 | 0.094022658 |
| chr12 | 1.19E+08 | 1.19E+08 | 343 | 0.012 | Taok3 | 543 | 0.030025023 | −0.061604516 | 0.024758284 | −0.060254613 |
| chr12 | 1.22E+08 | 1.22E+08 | 630 | 0.035 | Tmem120b | 33014 | 0.013695007 | −0.095305859 | 0.096425055 | −0.061082698 |
| chr12 | 1.25E+08 | 1.25E+08 | 470 | 0.006 | Ncor2 | 126377 | 0.102138472 | 0.049625685 | 0.098288525 | 0.072071702 |
| chr12 | 1.25E+08 | 1.25E+08 | 146 | 0.009 | Scarb1 | 14604 | 0.171709363 | 0.077782925 | 0.354573505 | 0.037757024 |
| chr12 | 1.26E+08 | 1.26E+08 | 234 | 0.035 | Aacs | 19023 | 0.115106488 | 0.074375332 | 0.004487949 | 0.068974009 |
| chr22 | 18298189 | 18298467 | 279 | 0.012 | Bid | 39778 | 0.036134946 | 0.088448521 | 0.013570732 | 0.072441841 |
| chr22 | 24761416 | 24761463 | 48 | 0.017 | Specc1l | 45885 | 0.344188283 | 0.014164191 | 0.067092193 | 0.044626658 |
| chr22 | 30278371 | 30278842 | 472 | 0.042 | Mtmr3 | 300 | 0.162418389 | 0.047787343 | 0.329332465 | 0.045700304 |
| chr22 | 36924935 | 36925290 | 356 | 0.012 | Eif3d | 0 | 0.033885498 | −0.073884507 | 0.106029245 | 0.058064788 |
| chr22 | 38472613 | 38473870 | 1258 | 0.005 | Slc16a8 | 3862 | 0.03773117 | −0.050802924 | 0.050792568 | 0.05132624 |
| chr22 | 40858582 | 40860665 | 2084 | 0.005 | Mkl1 | 58828 | 0.001384889 | −0.125891594 | 0.019491324 | −0.111849464 |
| chr22 | 43555410 | 43557147 | 1738 | 0.006 | Tspo | 8007 | 0.018177759 | −0.050771113 | 0.173972034 | −0.035722293 |
| chr22 | 45561031 | 45561107 | 77 | 0.044 | Nup50 | 708 | 0.264933651 | 0.033646427 | 0.020614385 | 0.09905398 |
| chr22 | 50955702 | 50956285 | 584 | 0.005 | Ncaph2 | 7477 | 0.069548977 | 0.071080994 | 0.058356976 | 0.041453832 |
| chr17 | 1220102 | 1220359 | 258 | 0.005 | Tusc5 | 32046 | 0.00498105 | 0.124949686 | 0.331277878 | −0.037317959 |
| chr17 | 7483300 | 7483645 | 346 | 0.029 | Cd68 | 288 | 0.004383267 | −0.086940803 | 0.004551923 | 0.051619704 |
| chr17 | 8027102 | 8027420 | 319 | 0.029 | Hes7 | 4 | 0.039741912 | −0.033411302 | 0.158273454 | −0.02601069 |
| chr17 | 8867824 | 8868379 | 556 | 0.005 | Pik3r5 | 611 | 0.128040105 | −0.057670787 | 0.458486372 | −0.038137455 |
| chr17 | 9950908 | 9951126 | 219 | 0.028 | Gas7 | 46063 | 0.208451228 | −0.047643312 | 0.065000979 | −0.064380479 |
| chr17 | 26646057 | 26646266 | 210 | 0.026 | Tmem97 | 0 | 0.009588159 | −0.219201828 | 0.041101816 | −0.168257023 |
| chr17 | 29150010 | 29150903 | 894 | 0.006 | Crlf3 | 749 | 0.064732777 | −0.026243112 | 0.054155807 | 0.070975564 |
| chr17 | 29641021 | 29641319 | 299 | 0.015 | Evi2b | 0 | 0.137955415 | −0.060964811 | 0.009073702 | −0.103736739 |
| chr17 | 34415897 | 34416118 | 222 | 0.026 | Ccl3 | 1024 | 0.001156209 | −0.090858161 | 0.043889966 | −0.052169682 |
| chr17 | 34417557 | 34418034 | 478 | 0.009 | Ccl3 | 51 | 0.001156209 | −0.090858161 | 0.043889966 | −0.052169682 |
| chr17 | 35299693 | 35299863 | 171 | 0.022 | Lhx1 | 5283 | 0.353425615 | 0.019486541 | 0.080261005 | 0.03668788 |
| chr17 | 40142289 | 40142472 | 184 | 0.032 | Dnajc7 | 27020 | 0.07140143 | 0.050557861 | 0.179317913 | −0.0406226 |
| chr17 | 45908521 | 45908890 | 370 | 0.026 | Mrpl10 | 3 | 0.158883651 | 0.046498288 | 0.152146362 | 0.048085714 |
| chr17 | 47819090 | 47819795 | 706 | 0.005 | 5730593 | 17777 | 0.007399875 | −0.102310745 | 0.007408441 | −0.126769783 |
| | | | | | F17Rik | | | | | |
| chr17 | 47863126 | 47864532 | 1407 | 0.022 | Myst2 | 1764 | 0.071136849 | 0.073246062 | 0.028307361 | 0.092132287 |
| chr17 | 48940751 | 48941296 | 546 | 0.006 | Tob1 | 2269 | 0.06118011 | 0.085294662 | 0.175848717 | −0.071444824 |
| chr17 | 56124678 | 56125091 | 414 | 0.005 | Sfrs1 | 19470 | 0.022776363 | −0.076070557 | 0.129254578 | 0.061583356 |
| chr17 | 56355449 | 56355594 | 146 | 0.044 | Mpo | 3658 | 0.038596305 | −0.060609612 | 0.084255863 | −0.04298877 |
| chr17 | 56490498 | 56491520 | 1023 | 0.005 | Hsf5 | 49038 | 0.022309925 | 0.109264262 | 0.047502101 | −0.070842093 |
| chr17 | 62204776 | 62206576 | 1801 | 0.006 | Ern1 | 891 | 0.076370686 | −0.096781936 | 0.146030662 | 0.057706948 |
| chr17 | 63541217 | 63541473 | 257 | 0.032 | Axin2 | 15769 | 0.020481163 | −0.049730423 | 0.106289649 | −0.042179775 |
| chr17 | 64448688 | 64449382 | 695 | 0.005 | Prkca | 120673 | 0.14072088 | −0.047904076 | 0.155558681 | −0.061898328 |
| chr17 | 66877696 | 66877766 | 71 | 0.044 | Abca8a | 53490 | 0.122227015 | −0.086695187 | 0.015035725 | 0.050854997 |
| chr17 | 73406576 | 73407600 | 1025 | 0.006 | Grb2 | 4125 | 0.110354272 | −0.048225572 | 0.011694515 | 0.073869847 |
| chr17 | 73630171 | 73630823 | 653 | 0.026 | Recql5 | 32121 | 0.006844407 | −0.104660956 | 0.002223662 | 0.207049308 |
| chr17 | 76375529 | 76375618 | 90 | 0.047 | Pgs1 | 567 | 0.160821825 | 0.071059014 | 0.26928162 | 0.039626158 |
| chr17 | 79419543 | 79420749 | 1207 | 0.006 | Bahcc1 | 705 | 0.017498542 | 0.105982501 | 0.024719301 | 0.042729251 |
| chr17 | 80050393 | 80050984 | 592 | 0.017 | Fasn | 4126 | 0.035750412 | −0.102212516 | 0.002367071 | 0.106729786 |
| chr17 | 80052124 | 80053059 | 936 | 0.009 | Fasn | 2706 | 0.00234477 | 0.110938993 | 0.045378089 | 0.090742194 |
| chr17 | 80416305 | 80416449 | 145 | 0.044 | Narf | 171 | 0.377968144 | 0.039260145 | 0.613152845 | −0.033064781 |
| chr5 | 10304741 | 10305342 | 602 | 0.009 | Cmbl | 2007 | 0.016968763 | −0.102322221 | 0.044958962 | −0.088428901 |
| chr5 | 32780268 | 32780484 | 217 | 0.043 | Npr3 | 53629 | 0.043125176 | −0.062892296 | 0.017355784 | −0.141018179 |
| chr5 | 34655547 | 34655935 | 389 | 0.029 | Rai14 | 1628 | 0.04433336 | 0.097710785 | 0.057769423 | 0.068167382 |
| chr5 | 34687673 | 34687965 | 293 | 0.029 | Rai14 | 23832 | 0.027202557 | 0.113639208 | 0.000316782 | 0.222487047 |
| chr5 | 36535659 | 36536198 | 540 | 0.009 | Slc1a3 | 70605 | 0.188674087 | −0.035795174 | 0.154389728 | −0.080028599 |
| chr5 | 55737286 | 55737497 | 212 | 0.033 | Ankrd55 | 157337 | 0.016894229 | 0.094145368 | 0.097086997 | 0.041114561 |
| chr5 | 64333579 | 64333820 | 242 | 0.022 | Sdccag10 | 205522 | 0.011515023 | 0.080503852 | 0.221219504 | 0.036053507 |
| chr5 | 66491448 | 66492361 | 914 | 0.005 | Cd180 | 251 | 0.010515394 | −0.065840584 | 0.032887588 | −0.052502908 |
| chr5 | 67585536 | 67585875 | 340 | 0.033 | Pik3r1 | 74846 | 0.027604432 | 0.069279842 | 0.05005877 | −0.054632625 |
| chr5 | 74244430 | 74245271 | 842 | 0.005 | 1700029 | 50707 | 0.051883314 | −0.077775325 | 0.163557371 | −0.029635614 |
| | | | | | F12Rik | | | | | |
| chr5 | 77803248 | 77803367 | 120 | 0.03 | Lhfpl2 | 118687 | 0.014339375 | −0.094089602 | 0.000121396 | −0.146102808 |
| chr5 | 79028795 | 79029354 | 560 | 0.032 | Papd4 | 98240 | 0.404746582 | −0.014708512 | 0.047724769 | 0.082858532 |
| chr5 | 1.07E+08 | 1.07E+08 | 257 | 0.011 | Efna5 | 96616 | 0.040259849 | 0.089666375 | 0.005299128 | 0.066779882 |
| chr5 | 1.07E+08 | 1.07E+08 | 438 | 0.006 | Efna5 | 64136 | 0.103233424 | 0.063036468 | 0.206316816 | −0.019221009 |
| chr5 | 1.08E+08 | 1.08E+08 | 299 | 0.011 | Fert2 | 30883 | 0.014590337 | −0.144368703 | 0.032578258 | 0.065941939 |
| chr5 | 1.34E+08 | 1.34E+08 | 288 | 0.022 | Sar1b | 0 | 0.19291661 | 0.155371226 | 0.336384668 | −0.016634489 |
| chr5 | 1.35E+08 | 1.35E+08 | 229 | 0.047 | BC027057 | 341 | 0.121578462 | −0.04209435 | 0.165450178 | −0.035528722 |
| chr5 | 1.42E+08 | 1.42E+08 | 525 | 0.006 | Spry4 | 26861 | 0.093448335 | 0.087272102 | 0.312019227 | 0.054293185 |
| chr5 | 1.48E+08 | 1.48E+08 | 406 | 0.006 | Adrb2 | 2176 | 0.005235308 | −0.099159277 | 0.055307519 | −0.100396049 |
| chr5 | 1.49E+08 | 1.49E+08 | 251 | 0.012 | Ppargc1b | 535 | 0.070181892 | 0.0958248 | 0.122617345 | 0.028066474 |
| chr5 | 1.49E+08 | 1.49E+08 | 691 | 0.006 | Csf1r | 714 | 0.008300488 | −0.087832678 | 0.011173575 | −0.049165072 |
| chr5 | 1.51E+08 | 1.51E+08 | 473 | 0.024 | Slc36a2 | 108 | 0.333779062 | 0.04041369 | 0.020229623 | 0.057497869 |
| chr5 | 1.7E+08 | 1.7E+08 | 263 | 0.011 | Kcnmb1 | 45521 | 1.42E−05 | −0.194462311 | 0.00669345 | −0.100721483 |
| chr5 | 1.76E+08 | 1.76E+08 | 688 | 0.012 | Hk3 | 182 | 0.178871723 | −0.050743362 | 0.233169223 | 0.039251216 |
| chr5 | 1.79E+08 | 1.79E+08 | 164 | 0.012 | Adamts2 | 35826 | 0.010600527 | −0.111699146 | 0.004506479 | 0.059363616 |
| chr7 | 1408433 | 1408951 | 519 | 0.006 | Micall2 | 64015 | 0.01030415 | 0.063819595 | 0.002222894 | 0.199369417 |
| chr7 | 8302335 | 8302647 | 313 | 0.012 | Ica1 | 149 | 0.852262755 | 0.008577271 | 0.210938579 | 0.051894475 |
| chr7 | 26460385 | 26460633 | 249 | 0.015 | Snx10 | 91887 | 0.017227015 | −0.112177321 | 0.020509591 | −0.104041417 |
| chr7 | 27201206 | 27201499 | 294 | 0.027 | Hoxa9 | 4830 | 0.000244 | −0.154545194 | 0.000896047 | −0.131389066 |
| chr7 | 27202553 | 27202993 | 441 | 0.044 | Hoxa9 | 3340 | 0.00772616 | −0.163900092 | 0.098968687 | −0.037616491 |
| chr7 | 27203073 | 27203466 | 394 | 0.008 | Hoxa9 | 2880 | 0.00772616 | −0.163900092 | 0.098968687 | −0.037616491 |
| chr7 | 30287772 | 30288094 | 323 | 0.009 | Znrf2 | 38759 | 0.502192871 | 0.028442713 | 0.197857941 | −0.076881409 |
| chr7 | 47672033 | 47673452 | 1420 | 0.006 | Tns3 | 24418 | 0.001650023 | 0.090694865 | 0.01767947 | 0.067112733 |
| chr7 | 50345715 | 50346835 | 1121 | 0.005 | Ikzf1 | 1486 | 0.000203584 | −0.123971624 | 0.000648778 | −0.128215817 |
| chr7 | 73624879 | 73625346 | 468 | 0.005 | Lat2 | 610 | 0.025835427 | −0.095097771 | 0.073432421 | 0.07910527 |
| chr7 | 75549990 | 75550275 | 286 | 0.03 | Por | 6204 | 0.240896483 | −0.019749676 | 0.228422084 | −0.026925827 |
| chr7 | 75957929 | 75958231 | 303 | 0.012 | Ywhag | 24237 | 0.014476618 | −0.079952002 | 0.001556626 | −0.148300136 |
| chr7 | 76828348 | 76828576 | 229 | 0.042 | Fgl2 | 528 | 0.032838026 | −0.069069628 | 0.072158041 | −0.095986623 |
| chr7 | 97500452 | 97501026 | 575 | 0.005 | Asns | 637 | 0.05303718 | 0.096679458 | 0.11633825 | 0.047365147 |
| chr7 | 1E+08 | 1E+08 | 283 | 0.012 | Trfr2 | 1124 | 0.074148968 | 0.074548168 | 0.048991726 | 0.082537723 |
| chr7 | 1.05E+08 | 1.05E+08 | 217 | 0.03 | Atxn7l1 | 167219 | 0.001087181 | 0.126246501 | 0.023129641 | −0.102921606 |
| chr7 | 1.07E+08 | 1.07E+08 | 526 | 0.005 | Prkar2b | 15286 | 0.001244862 | 0.088189068 | 0.052518879 | 0.057034208 |
| chr7 | 1.08E+08 | 1.08E+08 | 786 | 0.006 | Lamb1-1 | 27653 | 0.256006703 | 0.064722568 | 0.289143787 | 0.05643269 |
| chr7 | 1.08E+08 | 1.08E+08 | 362 | 0.005 | Lamb1-1 | 51183 | 0.020384645 | −0.090978916 | 0.010390466 | −0.114733938 |
| chr7 | 1.28E+08 | 1.28E+08 | 988 | 0.005 | Lep | 27192 | 0.193642885 | 0.043408718 | 0.372316108 | 0.037308041 |
| chr7 | 1.28E+08 | 1.28E+08 | 209 | 0.042 | Lep | 6068 | 0.177358186 | 0.052936735 | 0.08475912 | 0.050701004 |
| chr7 | 1.31E+08 | 1.31E+08 | 528 | 0.043 | Tsga13 | 193618 | 0.062198878 | 0.062047453 | 0.124462472 | 0.066968759 |
| chr7 | 1.31E+08 | 1.31E+08 | 306 | 0.012 | Podxl | 350 | 0.099786111 | −0.055238515 | 0.09342786 | 0.08650265 |
| chr7 | 1.4E+08 | 1.4E+08 | 1373 | 0.005 | Tbxas1 | 36 | 7.05E−05 | −0.203984492 | 0.012069378 | −0.094241458 |
| chr7 | 1.4E+08 | 1.4E+08 | 465 | 0.009 | Slc37a3 | 30231 | 0.219770505 | 0.023951494 | 0.103260316 | 0.079376392 |
| chr7 | 1.51E+08 | 1.51E+08 | 177 | 0.035 | Nub1 | 553 | 0.00124038 | 0.194317O77 | 0.000247585 | 0.144171977 |
| chr14 | 20898926 | 20899114 | 189 | 0.012 | Osgep | 31189 | 0.001031265 | −0.133171333 | 0.005996648 | −0.109023293 |
| chr14 | 21568858 | 21569390 | 533 | 0.035 | Zfp219 | 3747 | 0.227490337 | 0.046680964 | 0.083284561 | −0.049659277 |
| chr14 | 23788798 | 23789618 | 821 | 0.005 | Pabpn1 | 901 | 0.113051505 | −0.098799804 | 0.014235179 | 0.070986247 |
| chr14 | 31888216 | 31888303 | 88 | 0.026 | Heatr5a | 1354 | 0.051428286 | −0.077015105 | 0.074113648 | −0.052222637 |
| chr14 | 39737165 | 39737329 | 165 | 0.046 | Ctage5 | 2120 | 0.09171178 | 0.047880934 | 0.083113321 | −0.061483438 |
| chr14 | 55833724 | 55834573 | 850 | 0.012 | D14 | 26708 | 0.128314832 | 0.019852413 | 0.062240794 | −0.035332666 |
| | | | | | Ertd436e | | | | | |
| chr14 | 55834889 | 55835484 | 596 | 0.033 | D14 | 26166 | 0.128314832 | 0.019852413 | 0.062240794 | −0.035332666 |
| | | | | | Ertd436e | | | | | |
| chr14 | 74961318 | 74961491 | 174 | 0.022 | Npc2 | 1191 | 0.02971491 | −0.068992065 | 0.174558788 | −0.037000804 |
| chr14 | 75361059 | 75361571 | 513 | 0.033 | Dlst | 12923 | 0.002412449 | −0.165986778 | 0.006711909 | −0.148309561 |
| chr14 | 77791405 | 77791671 | 267 | 0.011 | Gstz1 | 10333 | 0.010968896 | 0.134177876 | 0.049884578 | 0.089298147 |
| chr14 | 81867674 | 81868031 | 358 | 0.015 | Ston2 | 37969 | 0.081282534 | −0.073907314 | 0.268537265 | 0.049810371 |
| chr14 | 89707672 | 89708616 | 945 | 0.009 | Foxn3 | 192777 | 0.005943992 | 0.082570716 | 0.097830571 | 0.051674454 |
| chr14 | 93117257 | 93117768 | 512 | 0.029 | Rin3 | 84784 | 0.003786645 | −0.112199089 | 0.01225759 | −0.071423943 |
| chr14 | 97180040 | 97180391 | 352 | 0.006 | Vrk1 | 72542 | 0.161683822 | 0.025082244 | 0.038464686 | 0.048180668 |
| chr14 | 1.01E+08 | 1.01E+08 | 749 | 0.006 | Evl | 81191 | 0.003391464 | −0.113839928 | 0.004730441 | −0.106016745 |
| chr9 | 14203697 | 14204081 | 385 | 0.005 | Nfib | 100379 | 0.001304379 | 0.122130904 | 0.009561952 | −0.121569828 |
| chr9 | 32457089 | 32457364 | 276 | 0.005 | Ddx58 | 33770 | 0.019332685 | 0.140039676 | 0.303952013 | −0.02997273 |
| chr9 | 36778847 | 36778989 | 143 | 0.033 | Pax5 | 252932 | 0.017411606 | −0.117459817 | 0.022346231 | −0.106487141 |
| chr9 | 93565879 | 93566460 | 582 | 0.006 | Syk | 11788 | 0.007420128 | 0.094453504 | 1.49E−05 | 0.197044812 |
| chr9 | 95726604 | 95726801 | 198 | 0.03 | Fgd3 | 0 | 0.000782954 | −0.190154032 | 0.106857929 | −0.053373774 |
| chr9 | 1.13E+08 | 1.13E+08 | 387 | 0.009 | Txn1 | 2744 | 0.153227162 | 0.059319005 | 0.063978233 | −0.069727536 |
| chr9 | 1.13E+08 | 1.13E+08 | 479 | 0.005 | Svep1 | 28635 | 0.635801168 | −0.026481336 | 0.32169114 | −0.045576333 |
| chr9 | 1.14E+08 | 1.14E+08 | 387 | 0.006 | Gng10 | 615 | 0.008616226 | −0.054665936 | 0.004046392 | 0.113837927 |
| chr9 | 1.16E+08 | 1.16E+08 | 318 | 0.033 | Rgs3 | 108843 | 0.034041424 | 0.053038017 | 0.076859759 | 0.032379635 |
| chr9 | 1.17E+08 | 1.17E+08 | 322 | 0.045 | Orm1 | 0 | 0.089241078 | 0.033000789 | 0.173649309 | 0.031700782 |
| chr9 | 1.17E+08 | 1.17E+08 | 468 | 0.006 | Akna | 2023 | 0.090278173 | 0.057770456 | 0.064277099 | 0.083163963 |
| chr9 | 1.18E+08 | 1.18E+08 | 333 | 0.016 | Tnfsf8 | 0 | 0.000933629 | −0.161745875 | 0.010526571 | −0.109511716 |
| chr9 | 1.3E+08 | 1.3E+08 | 369 | 0.006 | 9130404 | 41455 | 0.121434378 | −0.03946649 | 0.006459746 | 0.116243706 |
| | | | | | D14Rik | | | | | |
| chr9 | 1.31E+08 | 1.31E+08 | 610 | 0.005 | Spna2 | 45170 | 0.021422761 | 0.053623327 | 0.017081786 | −0.090287465 |
| chr9 | 1.33E+08 | 1.33E+08 | 208 | 0.03 | Fnbp1 | 1050 | 0.060174876 | −0.083590765 | 0.05641399 | 0.093572322 |
| chr13 | 29293918 | 29294030 | 113 | 0.032 | Slc46a3 | 979 | 0.234074293 | 0.075604237 | 0.130567277 | 0.047285533 |
| chr13 | 32780792 | 32781098 | 307 | 0.012 | Brca2 | 112608 | 0.477862977 | 0.019990114 | 0.189502768 | −0.03662977 |
| chr13 | 36049213 | 36050887 | 1675 | 0.005 | Mab2111 | 0 | 0.000513404 | −0.083527653 | 0.000333282 | −0.083898174 |
| chr13 | 40809925 | 40810619 | 695 | 0.009 | Foxo1 | 332765 | 0.449595212 | 0.027281116 | 0.026363464 | 0.067673717 |
| chr13 | 40943529 | 40943850 | 322 | 0.006 | Foxo1 | 248828 | 0.045248093 | 0.073827692 | 0.097759862 | −0.042986649 |
| chr13 | 44936911 | 44937231 | 321 | 0.012 | Tsc22d1 | 64627 | 0.071609449 | −0.039506342 | 0.022718659 | −0.050643262 |
| chr13 | 45564095 | 45564224 | 130 | 0.026 | 12000 | 323 | 0.004529161 | −0.179434382 | 0.048036648 | −0.105042022 |
| | | | | | 11I18Rik | | | | | |
| chr13 | 45694501 | 45694858 | 358 | 0.006 | Gtf2f2 | 0 | 0.004084029 | −0.071848765 | 0.04120081 | −0.076936457 |
| chr13 | 46751279 | 46752608 | 1330 | 0.005 | Lcp1 | 44287 | 0.014544051 | −0.091356551 | 0.02164575 | −0.098119319 |
| chr13 | 46753288 | 46754147 | 860 | 0.009 | Lcp1 | 42705 | 0.014544051 | −0.091356551 | 0.02164575 | −0.098119319 |
| chr13 | 49683338 | 49683473 | 136 | 0.044 | Fndc3a | 105975 | 0.149221767 | 0.029842321 | 0.20143602 | −0.044226379 |
| chr13 | 50204794 | 50205145 | 352 | 0.022 | Arl11 | 1201 | 0.044279538 | −0.107739618 | 0.046926469 | −0.048619668 |
| chr13 | 1.14E+08 | 1.14E+08 | 171 | 0.022 | Grtp1 | 347 | 0.229393431 | −0.053562829 | 0.153692182 | 0.096703406 |
| chr3 | 9911613 | 9911971 | 359 | 0.029 | Cidec | 7236 | 0.014015847 | 0.066336204 | 0.028881232 | 0.05617051 |
| chr3 | 15667550 | 15667785 | 236 | 0.011 | Btd | 13549 | 0.057143936 | −0.043614963 | 0.01757608 | −0.042154639 |
| chr3 | 15681784 | 15682347 | 564 | 0.006 | Btd | 23503 | 0.152744834 | −0.05127628 | 0.091948578 | 0.056366809 |
| chr3 | 24869934 | 24870287 | 354 | 0.008 | Rarb | 598099 | 0.029777881 | 0.046387087 | 0.111070939 | 0.109112789 |
| chr3 | 32412954 | 32413251 | 298 | 0.006 | Cmtm8 | 56143 | 0.172866571 | 0.03624256 | 0.165838952 | 0.039884397 |
| chr3 | 37907270 | 37907753 | 484 | 0.005 | Ctdspl | 4077 | 0.374453931 | 0.023449625 | 0.113248746 | 0.053198313 |
| chr3 | 38007994 | 38008396 | 403 | 0.009 | Ctdspl | 102518 | 0.031627285 | −0.085747522 | 0.080380047 | −0.058105565 |
| chr3 | 40499457 | 40499546 | 90 | 0.05 | Rpl14 | 656 | 0.026209488 | 0.061704304 | 0.17181901 | −0.03784743 |
| chr3 | 46036856 | 46037289 | 434 | 0.011 | Fyco1 | 42 | 0.013540094 | −0.234110654 | 0.107252986 | −0.152537306 |
| chr3 | 52445047 | 52445468 | 422 | 0.006 | Phf7 | 319 | 0.164356522 | 0.045244721 | 0.118265149 | −0.022978991 |
| chr3 | 52567262 | 52567405 | 144 | 0.042 | Nt5dc2 | 3223 | 0.130156467 | −0.037378327 | 0.009817152 | 0.115683951 |
| chr3 | 57262305 | 57262906 | 602 | 0.006 | Appl1 | 461 | 0.239989212 | −0.051311872 | 0.334632607 | 0.030190953 |
| chr3 | 65434405 | 65434559 | 155 | 0.046 | Magi1 | 143197 | 0.049562076 | −0.048552937 | 0.136872297 | −0.047379194 |
| chr3 | 66633695 | 66634011 | 317 | 0.032 | Lrig1 | 61174 | 0.087614339 | −0.038846936 | 0.028552309 | −0.066755725 |
| chr3 | 67049442 | 67049635 | 194 | 0.033 | Kbtbd8 | 655 | 0.159046189 | 0.057621246 | 0.054193913 | 0.06292396 |
| chr3 | 72870608 | 72870903 | 296 | 0.036 | Shq1 | 25788 | 0.026213972 | −0.070105607 | 0.020065667 | −0.074226033 |
| chr3 | 72896699 | 72897027 | 329 | 0.047 | Shq1 | 466 | 0.004251486 | −0.192497184 | 0.034505003 | −0.046382452 |
| chr3 | 1.13E+08 | 1.13E+08 | 1126 | 0.005 | Cd200r1 | 617 | 0.009566716 | −0.067486844 | 0.011117094 | −0.060831706 |
| chr3 | 1.13E+08 | 1.13E+08 | 568 | 0.008 | Boc | 31960 | 0.026237876 | 0.087180801 | 0.04824537 | 0.060003923 |
| chr3 | 1.24E+08 | 1.24E+08 | 224 | 0.015 | Umps | 89428 | 0.053370691 | −0.053070703 | 0.081051452 | 0.058941427 |
| chr3 | 1.25E+08 | 1.25E+08 | 235 | 0.033 | Itgb5 | 92104 | 0.112577227 | 0.052593515 | 0.011370824 | 0.112851305 |
| chr3 | 1.25E+08 | 1.25E+08 | 328 | 0.028 | Itgb5 | 91521 | 0.112577227 | 0.052593515 | 0.011370824 | 0.112851305 |
| chr3 | 1.25E+08 | 1.25E+08 | 206 | 0.033 | Itgb5 | 55665 | 0.33380135 | 0.051607354 | 0.214351388 | −0.046073299 |
| chr3 | 1.28E+08 | 1.28E+08 | 409 | 0.026 | Mgll | 29843 | 0.300572926 | 0.046883681 | 0.011379527 | −0.05814309 |
| chr3 | 1.34E+08 | 1.34E+08 | 358 | 0.015 | Rab6b | 767 | 0.012336117 | 0.053271596 | 0.021698788 | −0.10620353 |
| chr3 | 1.38E+08 | 1.38E+08 | 24 | 0.05 | Cep70 | 781 | 0.091346454 | 0.055603918 | 0.134506833 | 0.020105915 |
| chr3 | 1.41E+08 | 1.41E+08 | 1142 | 0.005 | Zbtb38 | 2118 | 0.01672593 | −0.121198507 | 0.065315651 | −0.09495155 |
| chr3 | 1.5E+08 | 1.5E+08 | 360 | 0.012 | 2810407 | 685 | 0.000503571 | −0.14397228 | 0.000438028 | −0.109932805 |
| | | | | | C02Rik | | | | | |
| chr3 | 1.51E+08 | 1.51E+08 | 283 | 0.012 | P2ry14 | 182 | 0.115599 | −0.047542803 | 0.226721227 | −0.036047052 |
| chr3 | 1.51E+08 | 1.51E+08 | 235 | 0.033 | P2ry12 | 44 | 0.314598291 | 0.057538343 | 0.068461354 | −0.089714333 |
| chr3 | 1.57E+08 | 1.57E+08 | 421 | 0.006 | Veph1 | 104131 | 0.000152002 | −0.188356272 | 0.004779003 | −0.130964675 |
| chr3 | 1.6E+08 | 1.6E+08 | 322 | 0.024 | Il12a | 917 | 0.510455058 | −0.037543221 | 0.26771562 | −0.062963536 |
| chr3 | 1.7E+08 | 1.7E+08 | 151 | 0.033 | Phc3 | 1137 | 0.06071042 | −0.081619325 | 0.290326978 | −0.020625775 |
| chr3 | 1.71E+08 | 1.71E+08 | 233 | 0.033 | Slc2a2 | 142633 | 0.246755268 | 0.062234751 | 0.37095251 | 0.043315768 |
| chr3 | 1.85E+08 | 1.85E+08 | 307 | 0.033 | Liph | 39515 | 0.11891308 | −0.025769524 | 0.062454307 | −0.078541633 |
| chr3 | 1.87E+08 | 1.87E+08 | 272 | 0.005 | Eif4a2 | 559 | 0.057501757 | 0.053855992 | 0.00334395 | −0.188930021 |
| chr3 | 1.87E+08 | 1.87E+08 | 352 | 0.005 | Bcl6 | 24573 | 0.183180484 | −0.023522408 | 0.061134987 | −0.076148653 |
| chr3 | 1.89E+08 | 1.89E+08 | 656 | 0.006 | Lpp | 579307 | 0.255225767 | 0.039438945 | 0.250766738 | −0.03871692 |
| chr3 | 1.96E+08 | 1.96E+08 | 1239 | 0.032 | Tctex1d2 | 18267 | 0.200280698 | −0.077940287 | 0.14598354 | −0.134146887 |
| chr3 | 1.96E+08 | 1.96E+08 | 1257 | 0.005 | Fbxo45 | 8610 | 0.051206131 | 0.032303493 | 0.076985143 | −0.062275775 |
| chr16 | 16123803 | 16124667 | 865 | 0.005 | Abcc1 | 39809 | 0.028137766 | 0.062463282 | 0.092616577 | 0.040749733 |
| chr16 | 20909935 | 20910137 | 203 | 0.015 | Dcun1d3 | 1217 | 0.085974868 | 0.056191121 | 0.030928297 | 0.107792016 |
| chr16 | 30455438 | 30455975 | 538 | 0.005 | Sephs2 | 1221 | 0.03253984 | −0.0577708 | 0.041044579 | −0.056633731 |
| chr16 | 46957730 | 46958679 | 950 | 0.005 | Gpt2 | 28479 | 0.031950761 | −0.034656493 | 0.077947645 | −0.045837574 |
| chr16 | 50059655 | 50059880 | 226 | 0.047 | Tmem188 | 410 | 0.063328638 | −0.103644757 | 0.056521478 | 0.078017874 |
| chr16 | 50715116 | 50715299 | 184 | 0.011 | 9130017 | 0 | 0.000204672 | −0.169680371 | 0.012381409 | −0.083634144 |
| | | | | | C17Rik | | | | | |
| chr16 | 55540360 | 55540833 | 474 | 0.006 | Mmp2 | 26328 | 0.012314177 | −0.055940936 | 0.083100844 | −0.036590525 |
| chr16 | 56137894 | 56138256 | 363 | 0.006 | Gnao1 | 85203 | 0.004769343 | 0.066758804 | 0.003215157 | 0.075936555 |
| chr16 | 57278607 | 57278995 | 389 | 0.022 | Arl2bp | 161 | 0.317131448 | 0.045084544 | 0.226855795 | −0.069460625 |
| chr16 | 69457347 | 69457490 | 144 | 0.044 | Cyb5b | 632 | 0.01244399 | 0.086794906 | 0.184650573 | 0.038753396 |
| chr16 | 79370715 | 79370926 | 212 | 0.024 | Wwox | 1022931 | 0.118336141 | 0.035616683 | 0.098991552 | −0.026484363 |
| chr16 | 83549673 | 83549934 | 262 | 0.044 | Cdh13 | 837835 | 0.092723296 | 0.034427003 | 0.079016286 | 0.033465629 |
| chr16 | 85376056 | 85376680 | 625 | 0.005 | Gse1 | 144355 | 0.182709459 | −0.023703324 | 0.056581861 | −0.087047513 |
| chr16 | 87903345 | 87904117 | 773 | 0.006 | BC048644 | 147 | 0.034324129 | −0.089302202 | 0.034224962 | −0.08017327 |
| chr16 | 88770501 | 88770848 | 348 | 0.012 | Rnf166 | 1390 | 0.043240371 | −0.073898435 | 0.137684355 | 0.089955892 |
| chr21 | 16057979 | 16058284 | 306 | 0.011 | Samsn1 | 144360 | 0.414561669 | −0.033457948 | 0.237872414 | 0.047423534 |
| chr21 | 33689979 | 33690246 | 268 | 0.033 | Mrap | 16472 | 0.149919203 | −0.039646958 | 0.115863135 | 0.02664686 |
| chr21 | 35448249 | 35449067 | 819 | 0.005 | Slc5a3 | 1968 | 0.003752114 | −0.121181521 | 0.006888168 | −0.093695803 |
| chr21 | 36398548 | 36398828 | 281 | 0.011 | Runx1 | 24726 | 0.000151796 | −0.15178274 | 0.001644441 | −0.119934451 |
| chr21 | 36420456 | 36421805 | 1350 | 0.005 | Runx1 | 0 | 4.31E−06 | −0.279855942 | 0.00055991 | −0.176657529 |
| chr21 | 37412563 | 37413036 | 474 | 0.006 | Setd4 | 17406 | 0.057139401 | 0.073919244 | 0.38706981 | 0.029344222 |
| chr21 | 39868683 | 39869555 | 873 | 0.01 | Erg | 71852 | 0.000107641 | −0.134357154 | 0.004244138 | −0.091090816 |
| chr21 | 40776034 | 40777497 | 1464 | 0.005 | Lcca51 | 33478 | 0.304302694 | 0.020796314 | 0.033232229 | 0.047454765 |
| chr18 | 2960647 | 2960946 | 300 | 0.03 | Lpin2 | 10199 | 0.226393124 | 0.074511207 | 0.221852968 | 0.065237008 |
| chr18 | 10457031 | 10457507 | 477 | 0.006 | Apcdd1 | 2177 | 0.093670381 | 0.042061125 | 0.13835125 | 0.026116591 |
| chr18 | 46277273 | 46277494 | 222 | 0.012 | Gm672 | 169837 | 0.060855394 | 0.080607141 | 0.002350005 | 0.17815372 |
| chr18 | 56809110 | 56809370 | 261 | 0.017 | Sec11c | 1867 | 0.047576474 | −0.102872363 | 0.081715262 | −0.086228022 |
| chr18 | 77440474 | 77440960 | 487 | 0.006 | Ctdp1 | 628 | 0.124164823 | −0.03050826 | 0.022559577 | −0.103251482 |
| chr19 | 7733678 | 7734230 | 553 | 0.026 | Retn | 0 | 0.017884245 | −0.067224475 | 0.127563827 | 0.037882218 |
| chr19 | 12920962 | 12921262 | 301 | 0.026 | Rnaseh2a | 5983 | 0.070335289 | −0.037572965 | 0.02018467 | −0.043792759 |
| chr19 | 15574480 | 15574831 | 352 | 0.046 | A430107 | 535 | 0.019917347 | −0.119050573 | 0.084596055 | 0.041823459 |
| | | | | | D22Rik | | | | | |
| chr19 | 17669572 | 17669647 | 76 | 0.017 | Glt25d1 | 751 | 0.503151826 | −0.027583264 | 0.401626335 | 0.039375052 |
| chr19 | 18508116 | 18508457 | 342 | 0.042 | Lrrc25 | 57 | 0.155971856 | −0.0272848 | 0.017410174 | 0.051164222 |
| chr19 | 18682314 | 18682390 | 77 | 0.022 | Uba52 | 309 | 0.137872609 | 0.056746254 | 0.238786636 | 0.062029191 |
| chr19 | 40750567 | 40750963 | 397 | 0.015 | Akt2 | 21904 | 0.00536166 | 0.105072993 | 0.026742224 | −0.064631594 |
| chr19 | 42830523 | 42830820 | 298 | 0.017 | Tmem145 | 11527 | 0.017467435 | −0.125098373 | 0.016096425 | 0.174781541 |
| chr19 | 42912089 | 42912557 | 469 | 0.026 | Lipe | 4500 | 0.063280265 | −0.066215746 | 0.080255544 | 0.04966715 |
| chr19 | 45225646 | 45225969 | 324 | 0.012 | Ceacam16 | 16447 | 0.096953319 | 0.111422729 | 0.082741162 | −0.044333143 |
| chr19 | 49841249 | 49841895 | 647 | 0.009 | Cd37 | 2686 | 0.027967301 | 0.091393575 | 0.000388475 | 0.135513451 |
| chr19 | 51645546 | 51714605 | 69060 | 0.006 | Cd33 | 0 | 0.026484673 | −0.072463543 | 0.042378002 | 0.037094866 |
| chr19 | 54876429 | 54876765 | 337 | 0.03 | Lair1 | 0 | 0.026489679 | −0.097348731 | 0.000733429 | −0.144786239 |
| chr11 | 6267881 | 6268720 | 840 | 0.012 | Cnga4 | 4830 | 0.001427368 | 0.142949402 | 0.095175091 | 0.049578861 |
| chr11 | 10316893 | 10317243 | 351 | 0.035 | Adm | 11261 | 0.015285684 | −0.079842283 | 0.074089008 | −0.059197721 |
| chr11 | 10324978 | 10325218 | 241 | 0.033 | Adm | 1249 | 0.006833463 | −0.076195352 | 0.022076563 | −.065313457 |
| chr11 | 13359375 | 13359540 | 166 | 0.042 | Arntl | 52507 | 0.287397995 | 0.033110364 | 0.151330045 | 0.046863197 |
| chr11 | 14563296 | 14563419 | 124 | 0.042 | Psma1 | 16958 | 0.268961707 | 0.042292388 | 0.098957367 | −0.092435765 |
| chr11 | 18127092 | 18127637 | 546 | 0.011 | Saa3 | 5006 | 0.22057406 | 0.022446951 | 0.1205803 | 0.031998649 |
| chr11 | 27490752 | 27491523 | 772 | 0.01 | Lgr4 | 2692 | 0.020887869 | −0.168418694 | 0.111469562 | −0.107221087 |
| chr11 | 32852321 | 32852814 | 494 | 0.009 | Prrg4 | 759 | 0.181458128 | 0.058426216 | 0.201958387 | 0.043459733 |
| chr11 | 47291136 | 47291346 | 211 | 0.026 | Madd | 246 | 0.117725556 | −0.06055027 | 0.274985803 | 0.058660731 |
| chr11 | 47415025 | 47415403 | 379 | 0.006 | Sfpi1 | 12317 | 0.005612808 | −0.09283909 | 0.020093253 | −0.111136916 |
| chr11 | 57067776 | 57068975 | 1200 | 0.005 | Tnks1bp1 | 20866 | 0.031735717 | −0.108159774 | 0.00274766 | −0.100677313 |
| chr11 | 63528336 | 63528808 | 473 | 0.006 | 27000 | 4270 | 0.009374816 | 0.075120856 | 0.019783585 | 0.057464615 |
| | | | | | 81O15Rik | | | | | |
| chr11 | 64544695 | 64545147 | 453 | 0.012 | Sf1 | 988 | 0.074007346 | 0.080573957 | 0.05951643 | −0.060992111 |
| chr11 | 66673568 | 66673798 | 231 | 0.043 | Pcx | 24624 | 0.049344564 | 0.089939048 | 0.126682615 | 0.044461048 |
| chr11 | 67036383 | 67036711 | 329 | 0.029 | Adrbk1 | 2011 | 0.000861836 | −0.127008445 | 0.093207488 | −0.050972176 |
| chr11 | 67165584 | 67166226 | 643 | 0.009 | Ppp1ca | 2700 | 0.038535186 | 0.032864959 | 0.003821021 | 0.093715644 |
| chr11 | 73692113 | 73692198 | 86 | 0.028 | Ucp2 | 1517 | 0.048032778 | −0.086506376 | 0.07985297 | −0.086876483 |
| chr11 | 76337568 | 76338271 | 704 | 0.006 | 2210018 | 121609 | 0.052017168 | −0.077393197 | 0.162986482 | 0.044929939 |
| | | | | | M11Rik | | | | | |
| chr11 | 1.18E+08 | 1.18E+08 | 245 | 0.022 | Il10ra | 1315 | 0.000608402 | −0.109964715 | 0.020983984 | −0.060170029 |
| chr11 | 1.21E+08 | 1.21E+08 | 217 | 0.033 | Tecta | 14379 | 0.070233309 | −0.032303992 | 0.02525565 | −0.038009449 |
| chr11 | 1.23E+08 | 1.23E+08 | 571 | 0.015 | Bsx | 2351 | 0.034711309 | 0.095100232 | 0.010189507 | −0.075016245 |
| chr15 | 37173455 | 37174113 | 659 | 0.026 | Meis2 | 210978 | 0.028601312 | −0.075724131 | 0.011445318 | −0.072710637 |
| chr15 | 52404555 | 52404986 | 432 | 0.022 | Bcl2110 | 0 | 0.115604037 | 0.071796217 | 0.352538451 | 0.03906306 |
| chr15 | 52820075 | 52820295 | 221 | 0.022 | Myo5a | 914 | 0.694329386 | 0.024475175 | 0.302911419 | −0.054874844 |
| chr15 | 61136559 | 61136807 | 249 | 0.024 | Rora | 381025 | 0.001200488 | −0.123028637 | 0.091815399 | −0.061985343 |
| chr15 | 62343394 | 62344034 | 641 | 0.005 | Vps13c | 7451 | 0.214289764 | −0.064912798 | 0.02432572 | −0.11424508 |
| chr15 | 63571085 | 63571484 | 400 | 0.006 | Aph1c | 1160 | 0.284299059 | −0.051881135 | 0.009652533 | 0.07466307 |
| chr15 | 69426262 | 69426542 | 281 | 0.035 | Glce | 82581 | 0.018055549 | −0.112554107 | 0.053930571 | −0.090359063 |
| chr15 | 69708054 | 69708318 | 265 | 0.036 | Kif23 | 1052 | 0.020715438 | 0.049924329 | 0.000803319 | 0.072074773 |
| chr15 | 77478505 | 77479038 | 534 | 0.033 | C230081 | 153180 | 0.231666602 | 0.070100851 | 0.018574335 | 0.089551919 |
| | | | | | A13Rik | | | | | |
| chr15 | 79471213 | 79471368 | 156 | 0.05 | Rasgrf1 | 77403 | 0.074622439 | −0.035916955 | 0.20567207 | −0.042072017 |
| chr15 | 89977287 | 89977470 | 184 | 0.044 | Rhcg | 47905 | 0.00877423 | −0.073352554 | 0.185544683 | 0.080818625 |
| chr15 | 90701174 | 90701775 | 602 | 0.009 | Sema4b | 29547 | 0.085843566 | 0.0589646 | 0.10894982 | 0.052268715 |
| chr15 | 96902600 | 96903113 | 514 | 0.005 | Nr2f2 | 27667 | 4.02E−05 | −0.13012121 | 0.001088048 | −0.175874276 |
| chr15 | 96905568 | 96906197 | 630 | 0.006 | Nr2f2 | 30696 | 0.000917372 | −0.151366539 | 7.85E−06 | −0.184271449 |
| chr15 | 96907528 | 96907914 | 387 | 0.022 | Nr2f2 | 32822 | 0.000917372 | −0.151366539 | 7.85E−06 | −0.184271449 |
| chr20 | 1879865 | 1880324 | 460 | 0.005 | Sirpa | 3655 | 0.001414611 | 0.163898814 | 0.28961536 | 0.043137468 |
| chr20 | 3527981 | 3528426 | 446 | 0.036 | Atrn | 39569 | 0.147326039 | 0.084492638 | 0.260585943 | 0.054837666 |
| chr20 | 3746584 | 3746692 | 109 | 0.011 | 1700037 | 1278 | 0.139490039 | −0.044307576 | 0.18975352 | −0.035203827 |
| | | | | | H04Rik | | | | | |
| chr20 | 18269464 | 18269677 | 214 | 0.029 | 6330439 | 67696 | 0.004138053 | 0.11442861 | 0.002225491 | 0.05937174 |
| | | | | | K17Rik | | | | | |
| chr20 | 25033629 | 25034262 | 634 | 0.005 | Acss1 | 4489 | 0.035246291 | 0.053724766 | 0.043383222 | 0.079822794 |
| chr20 | 25251494 | 25252151 | 658 | 0.005 | Pygb | 21579 | 0.00912653 | 0.050518875 | 0.014407649 | 0.040195217 |
| chr20 | 30258398 | 30258882 | 485 | 0.009 | Bcl2l1 | 45565 | 0.062783463 | 0.088332976 | 0.038981244 | −0.084371149 |
| chr20 | 33466246 | 33466396 | 151 | 0.015 | Acss2 | 1292 | 0.135099492 | 0.049631059 | 0.388269862 | 0.026833312 |
| chr20 | 35233479 | 35233726 | 248 | 0.012 | 1110008 | 517 | 0.178100009 | −0.054084787 | 0.462149615 | 0.033702996 |
| | | | | | F13Rik | | | | | |
| chr20 | 36104587 | 36105045 | 459 | 0.005 | Blcap | 41702 | 0.155524754 | −0.044843164 | 0.167318597 | −0.047607368 |
| chr20 | 36823215 | 36823538 | 324 | 0.026 | D630003 | 54558 | 0.060042606 | −0.026596859 | 0.035622337 | −0.035290347 |
| | | | | | M21Rik | | | | | |
| chr20 | 39314354 | 39315324 | 971 | 0.006 | Mafb | 2438 | 0.016335516 | −0.073400089 | 0.014954149 | −0.068027443 |
| chr20 | 43953416 | 43953593 | 178 | 0.026 | Sdc4 | 19786 | 0.222068845 | −0.027653814 | 0.067644188 | −0.0556007 |
| chr20 | 47803610 | 47804113 | 504 | 0.006 | Stau1 | 669 | 0.04857818 | −0.099555153 | 0.316221226 | −0.052500125 |
| chr20 | 47839439 | 47839685 | 247 | 0.012 | Ddx27 | 4251 | 0.156286748 | −0.02991861 | 0.047684049 | −0.043391968 |
| chr20 | 48924232 | 48924768 | 537 | 0.022 | Cebpb | 87922 | 0.004243497 | 0.096121482 | 0.078455137 | 0.076853806 |
| chr20 | 56135219 | 56135721 | 503 | 0.006 | Pck1 | 452 | 0.008351783 | 0.073282322 | 0.046530677 | −0.048857478 |
| chr20 | 56135935 | 56137320 | 1386 | 0.005 | Pck1 | 0 | 0.003187762 | 0.087199606 | 0.040124916 | 0.040920345 |
| chr20 | 56138648 | 56138937 | 290 | 0.016 | Pck1 | 2893 | 0.003187762 | 0.087199606 | 0.040124916 | 0.040920345 |
| chr20 | 56139464 | 56139982 | 519 | 0.009 | Pck1 | 4078 | 0.003187762 | 0.087199606 | 0.040124916 | 0.040920345 |
| chr20 | 58512538 | 58512948 | 411 | 0.03 | Ppp1r3d | 2200 | 0.099138641 | 0.056386238 | 0.054855161 | 0.084754804 |
| chr4 | 3295253 | 3295496 | 244 | 0.022 | Rgs12 | 379 | 0.21813933 | 0.078202348 | 0.090432956 | 0.036055255 |
| chr4 | 38858508 | 38859749 | 1242 | 0.008 | Tlr6 | 90 | 0.003242039 | −0.125613648 | 0.050924237 | −0.072456987 |
| chr4 | 39408228 | 39410160 | 1933 | 0.005 | Klb | 0 | 0.044594542 | −0.072267154 | 0.237736274 | −0.045043502 |
| chr4 | 84034389 | 84034401 | 13 | 0.015 | Plac8 | 1423 | 0.000415556 | −0.184889567 | 0.009648694 | −0.115334801 |
| chr4 | 86398976 | 86399113 | 138 | 0.047 | Arhgap24 | 2326 | 0.063282407 | 0.060784671 | 0.005230217 | 0.106808245 |
| chr4 | 87672032 | 87672388 | 357 | 0.006 | Ptpn13 | 116187 | 0.100709592 | −0.079778073 | 0.040493552 | −0.100648092 |
| chr4 | 1.09E+08 | 1.09E+08 | 352 | 0.005 | Papss1 | 66631 | 0.052559898 | −0.072581024 | 0.041710937 | −0.091658981 |
| chr4 | 1.09E+08 | 1.09E+08 | 207 | 0.024 | Hadh | 18992 | 0.04453389 | 0.055177969 | 0.171434844 | 0.022386383 |
| chr4 | 1.14E+08 | 1.14E+08 | 773 | 0.005 | Larp7 | 232193 | 0.027308541 | −0.078932185 | 0.053867885 | −0.064062514 |
| chr4 | 1.2E+08 | 1.2E+08 | 206 | 0.005 | Sec24d | 78238 | 0.1507209 | 0.081634037 | 0.281930451 | 0.044129842 |
| chr4 | 1.21E+08 | 1.21E+08 | 181 | 0.046 | Pde5a | 1646 | 0.051707622 | 0.108026119 | 0.296110272 | 0.057968662 |
| chr4 | 1.29E+08 | 1.29E+08 | 301 | 0.012 | Larp2 | 107364 | 0.046803004 | −0.037213593 | 0.050041702 | −0.080257748 |
| chr4 | 1.29E+08 | 1.29E+08 | 1867 | 0.005 | Larp2 | 151187 | 0.145876681 | 0.034008826 | 0.114284609 | 0.061117539 |
| chr4 | 1.84E+08 | 1.84E+08 | 553 | 0.006 | Cldn22 | 0 | 0.04719212 | 0.079883969 | 0.009105591 | −0.067573859 |
| chr4 | 1.85E+08 | 1.85E+08 | 511 | 0.005 | Stox2 | 31 | 0.010583934 | 0.096433022 | 0.081507317 | −0.046430409 |
| chr4 | 1.85E+08 | 1.85E+08 | 374 | 0.006 | Irf2 | 112511 | 0.044240008 | −0.068515138 | 0.017466763 | −0.083560547 |
| chr4 | 1.86E+08 | 1.86E+08 | 143 | 0.005 | Acsl1 | 4433 | 0.21397322 | 0.051362717 | 0.241807049 | −0.054203499 |
| chr4 | 1.86E+08 | 1.86E+08 | 1136 | 0.006 | Acsl1 | 1879 | 0.207983162 | −0.070745646 | 0.606290022 | −0.027547768 |
| chr4 | 1.86E+08 | 1.86E+08 | 766 | 0.035 | Acsl1 | 55713 | 0.174978393 | 0.035307295 | 0.122741315 | −0.034136668 |

This table lists the 497 mouse DMRs mappable onto the human chromosome and with 5 kb of a human probe. Listed are the genomic coordinates and width for each mouse differentially methylated region (DMR), q-values for the mouse DMRs derived from false discovery rate (see methods, qval), the gene symbol nearest gene to the mouse DMR, the p-values for the corresponding changes in human obesity and surgery, and the slopes for the methylation change for both human obesity and surgery.

TABLE 8
Enrichment of cross-species DMRs over DIAGRAM
GWAS loci, related to FIG. 3.

| DIAGRAM | | Number of | | Number of | |
|---|---|---|---|---|---|
| | # | GWAS | | GWAS | |
| Cutoff | Loci | Adipose | Islet | Adipose | Islet |
| p < 5e−08 | 18 | 2 (0.017) | 0 (0.292) | 1 (0.0113) | 0 (0.108) |
| p < 1e−07 | 20 | 2 (0.025) | 0 (0.329) | 1 (0.0154) | 0 (0.121) |
| p < 1e−06 | 35 | 3 (0.016) | 1 (0.119) | 1 (0.0376) | 0 (0.179) |
| p < 1e−05 | 215 | 6 (0.005) | 2 (0.119) | 2 (0.0253) | 1 (0.058) |
| p < 1e−04 | 428 | 9 (0.209) | 5 (0.239) | 3 (0.192) | 2 (0.166) |
| p < 1e−03 | 1601 | 24 (0.48) | 12 (0.600) | 6 (0.597) | 7 (0.086) |
| p < 1e−02 | 8353 | 77 (0.336) | 39 (0.560) | 27 (0.119) | 12 (0.573) |

This table summarizes the number and significance of overlaps of cross-species conserved adipose and pancreatic islet loci with DIAGRAM GWAS LD-blocks associated with SNPs at varying levels of significance as indicated by the cutoff column.

TABLE 9
Cross-species, directionally consistent DMRs that overlap with DIAGRAM T2D GWAS loci, related to Table 2.

| | | | | | | | | | Human | DIAGRAM | |
|---|---|---|---|---|---|---|---|---|---|---|---|
| Mouse | Mouse | | Mouse | Mouse | | | | Nearest | Pval | p-value of | |
| DMR | DMR | Mouse | DMR | DMR | | | Nearest | Gene | (for | nearest | DIAGRAM |
| Chr | start | DMR end | width | value | pval | qval | Gene | Dist | T2D) | GWAS loci | SNP |
| chr3 | 97536603 | 97536878 | 276 | 0.093611 | 0.001563 | 0.012831 | Arl6 | 34177 | 0.01811 | 0.00069 | rs17302349 |
| chr1 | 1.47E+08 | 1.47E+08 | 1014 | 0.079418 | 6.51E−05 | 0.002028 | Bcl9 | 0 | 0.018785 | 0.00042 | rs7512513 |
| chr2 | 1.7E+08 | 1.7E+08 | 721 | 0.080218 | 6.51E−05 | 0.002028 | G6pc2 | 0 | 0.005552 | 0.0034 | rs16856159 |
| chr11 | 17405939 | 17406583 | 645 | 0.080153 | 0.001497 | 0.012831 | Kcnj11 | 3320 | 0.011108 | 4.40E−06 | rs5215 |
| chr11 | 17406741 | 17407082 | 342 | 0.077431 | 0.00293 | 0.017074 | Kcnj11 | 2900 | 0.011108 | 4.40E−06 | rs5215 |
| chr11 | 17407373 | 17408268 | 896 | 0.081309 | 6.51E−05 | 0.002028 | Kcnj11 | 1740 | 0.001281 | 4.40E−06 | rs5215 |
| chr11 | 27491277 | 27491838 | 562 | −0.07805 | 0.000911 | 0.010693 | Lgr4 | 2417 | 0.045056 | 0.0077 | rs7945211 |
| chr14 | 77511555 | 77511688 | 134 | −0.08141 | 0.016081 | 0.045395 | 6430527 | 15082 | 0.024935 | 0.0029 | rs17752640 |
| | | | | | | | G18Rik | | | | |
| chr12 | 39297915 | 39298205 | 291 | −0.07128 | 0.004232 | 0.020119 | Cpne8 | 926 | 0.008167 | 1.60E−05 | rs10506132 |
| chr5 | 1.23E+08 | 1.23E+08 | 369 | 0.073114 | 0.001953 | 0.013892 | Csnk1g3 | 263645 | 0.010391 | 0.00051 | rs7708937 |

Similar to Table 3, this table lists pancreatic islet DMRs that are significant across species, directionally consistent, and overlap with DIAGRAM T2D LD blocks associated with nominally significant SNPs.

TABLE 10
Overlapping methylation change and adipose enhancer regions, related to Table 2.

| | | | | Mouse_{—} | Human_{—} | Human_{—} | | distToNearest |
|---|---|---|---|---|---|---|---|---|
| | | | | Nearest_{—} | Obesity_{—} | Surgery_{—} | distToNearest | AdiposeSuper |
| Mouse_chr | Mouse_start | Mouse_end | Mouse_Qval | Gene | Pval | Pval | AdiposeEnhancer | Enhancer |
| chr1 | 19804495 | 19805076 | 0.005 | Capzb | 0.009585 | 0.080255 | 0 | 0 |
| chr1 | 48351513 | 48351750 | 0.011 | OTTMUSG | 0.000272 | 0.105293 | −546242 | 3080210 |
| | | | | 00000008561 | | | | |
| chr1 | 64061530 | 64062519 | 0.006 | Pgm2 | 0.023275 | 0.223954 | 0 | 872196 |
| chr1 | 1.11E+08 | 1.11E+08 | 0.009 | Kcna3 | 0.00083 | 0.026296 | 199363 | 505540 |
| chr1 | 1.14E+08 | 1.14E+08 | 0.006 | Ptpn22 | 8.28E−05 | 0.111419 | −13238 | −2664547 |
| chr1 | 1.14E+08 | 1.14E+08 | 0.005 | Ptpn22 | 8.28E−05 | 0.111419 | −14457 | −2665766 |
| chr1 | 1.14E+08 | 1.14E+08 | 0.006 | Ptpn22 | 8.28E−05 | 0.111419 | −15365 | −2666674 |
| chr1 | 1.5E+08 | 1.5E+08 | 0.012 | Plekho1 | 0.011516 | 0.092524 | 0 | −140802 |
| chr1 | 1.51E+08 | 1.51E+08 | 0.005 | Tnfaip8l2 | 2.58E−05 | 0.043103 | −10710 | 123288 |
| chr1 | 1.61E+08 | 1.61E+08 | 0.005 | Cd48 | 0.037991 | 0.058607 | −131593 | 479879 |
| chr1 | 1.61E+08 | 1.61E+08 | 0.005 | Arhgap30 | 4.80E−09 | 0.010337 | 0 | 123016 |
| chr1 | 1.61E+08 | 1.61E+08 | 0.006 | Fcgr3 | 0.002653 | 0.017223 | 23356 | −301163 |
| chr1 | 1.93E+08 | 1.93E+08 | 0.006 | Rgs1 | 0.015727 | 0.016068 | −14758 | −8658971 |
| chr1 | 1.99E+08 | 1.99E+08 | 0.005 | Ptprc | 0.018214 | 0.001093 | 16327 | 2807046 |
| chr1 | 2.12E+08 | 2.12E+08 | 0.012 | Nek2 | 0.012206 | 0.061637 | −55976 | 531270 |
| chr2 | 16084776 | 16085714 | 0.024 | Mycn | 0.026463 | 0.020034 | −238915 | 703959 |
| chr2 | 47216781 | 47216960 | 0.024 | Ttc7 | 0.00172 | 0.177315 | 0 | 0 |
| chr2 | 62426656 | 62427018 | 0.012 | B3gnt2 | 0.021727 | 0.007112 | −588 | 494136 |
| chr2 | 67625367 | 67625994 | 0.011 | Etaa1 | 0.014192 | 0.057427 | −136215 | 789174 |
| chr2 | 68592241 | 68593052 | 0.005 | Plek | 0.000418 | 0.001208 | 0 | 45321 |
| chr2 | 68961351 | 68961704 | 0.012 | Arhgap25 | 0.000287 | 0.009437 | 28829 | −281076 |
| chr2 | 68962521 | 68963097 | 0.005 | Arhgap25 | 0.000287 | 0.009437 | 27436 | −282246 |
| chr2 | 1.01E+08 | 1.01E+08 | 0.03 | Lonrf2 | 0.037745 | 0.308483 | 49822 | 1884353 |
| chr2 | 1.45E+08 | 1.45E+08 | 0.011 | Zeb2 | 0.013433 | 0.433484 | 0 | −129373 |
| chr2 | 1.58E+08 | 1.58E+08 | 0.005 | Pscdbp | 0.003846 | 0.009592 | 53777 | −969584 |
| chr2 | 1.58E+08 | 1.58E+08 | 0.005 | Pscdbp | 0.003846 | 0.009592 | 52287 | −971607 |
| chr2 | 1.77E+08 | 1.77E+08 | 0.033 | Hoxd3 | 0.007824 | 0.00978 | −17061 | −2094046 |
| chr6 | 1608907 | 1609124 | 0.033 | Foxc1 | 0.021499 | 0.02655 | 2698 | 1409463 |
| chr6 | 26595986 | 26596063 | 0.044 | Abt1 | 0.001774 | 0.029453 | −22946 | −1872832 |
| chr6 | 75913118 | 75913414 | 0.011 | Col12a1 | 0.039965 | 0.018634 | 0 | 12455722 |
| chr6 | 79922240 | 79922533 | 0.009 | Hmgn3 | 0.031224 | 0.133123 | 19305 | 8446603 |
| chr6 | 1.08E+08 | 1.08E+08 | 0.011 | Scml4 | 0.000254 | 0.001184 | 128176 | 732446 |
| chr6 | 1.64E+08 | 1.64E+08 | 0.005 | Qk | 0.005485 | 0.039999 | 0 | 0 |
| chr8 | 1715132 | 1715419 | 0.012 | Cln8 | 0.020312 | 0.19778 | 0 | 5531120 |
| chr8 | 41907137 | 41907600 | 0.006 | Myst3 | 0.033952 | 0.032882 | 0 | −3240680 |
| chr8 | 66701734 | 66702089 | 0.006 | Pde7a | 0.000239 | 0.022706 | 50510 | −4064608 |
| chr8 | 70588403 | 70588535 | 0.047 | Slco5a1 | 0.037372 | 0.027051 | −70101 | 6994966 |
| chr8 | 79577640 | 79577971 | 0.012 | 3110050N22Rik | 0.027335 | 0.01307 | −49109 | −1965288 |
| chr8 | 1.29E+08 | 1.29E+08 | 0.026 | Myc | 0.029892 | 0.01329 | −1615 | 2002110 |
| chr8 | 1.31E+08 | 1.31E+08 | 0.03 | 0910001A06Rik | 0.006433 | 0.053705 | 0 | 0 |
| chr8 | 1.45E+08 | 1.45E+08 | 0.012 | AA409316 | 0.02697 | 0.004606 | −100509 | −1E+07 |
| chr10 | 6190543 | 6191242 | 0.006 | Rbm17 | 0.017146 | 0.019469 | 0 | 0 |
| chr10 | 97144444 | 97145224 | 0.005 | Sorbs1 | 0.027219 | 0.001956 | 0 | 0 |
| chr10 | 97517128 | 97517433 | 0.032 | Entpd1 | 0.026474 | 0.061996 | −14493 | −308852 |
| chr10 | 98416374 | 98417030 | 0.006 | Pik3ap1 | 0.002274 | 0.11671 | 15807 | −1208098 |
| chr10 | 1.05E+08 | 1.05E+08 | 0.005 | As3mt | 0.004186 | 0.018854 | −53262 | −53262 |
| chr10 | 1.05E+08 | 1.05E+08 | 0.026 | Sh3pxd2a | 0.000644 | 0.000123 | 0 | 229078 |
| chr10 | 1.15E+08 | 1.15E+08 | 0.035 | Tcf7l2 | 0.000356 | 0.353841 | 0 | 0 |
| chr10 | 1.15E+08 | 1.15E+08 | 0.009 | Tcf7l2 | 0.001798 | 0.008486 | 0 | 0 |
| chr10 | 1.3E+08 | 1.3E+08 | 0.005 | Ptpre | 0.006559 | 0.022389 | 0 | 1885307 |
| chr10 | 1.34E+08 | 1.34E+08 | 0.046 | Bnip3 | 0.032971 | 0.02267 | 0 | −2028713 |
| chr10 | 1.35E+08 | 1.35E+08 | 0.006 | 6430531B16Rik | 0.047166 | 0.027803 | 0 | −3307728 |
| chr12 | 1663136 | 1663829 | 0.005 | Wnt5b | 0.043716 | 0.049488 | 21029 | 102052 |
| chr12 | 7060921 | 7061200 | 0.032 | Ptpn6 | 0.000294 | 0.013488 | 501 | −578219 |
| chr12 | 8275965 | 8276317 | 0.026 | Clec4a4 | 0.013804 | 0.010436 | 32585 | −1793263 |
| chr12 | 15111717 | 15112453 | 0.005 | Arhgdib | 0.009564 | 0.029894 | 0 | 1386708 |
| chr12 | 26392741 | 26393036 | 0.031 | Sspn | 0.032866 | 0.136648 | 0 | −111890 |
| chr12 | 62658334 | 62658491 | 0.047 | Usp15 | 0.01282 | 0.018703 | 272793 | −2525517 |
| chr12 | 69140644 | 69140871 | 0.022 | Slc35e3 | 0.002949 | 0.174568 | −62479 | 119374 |
| chr12 | 75874872 | 75875171 | 0.005 | Glipr1 | 0.028933 | 0.133593 | 0 | −3198773 |
| chr12 | 95000081 | 95001986 | 0.026 | Tmcc3 | 0.004387 | 0.167216 | 6592 | −1200387 |
| chr12 | 1.03E+08 | 1.03E+08 | 0.005 | Pmch | 0.00435 | 0.264568 | −49193 | 173265 |
| chr12 | 1.04E+08 | 1.04E+08 | 0.017 | Tdg | 0.005058 | 0.05672 | −1480 | −1484292 |
| chr12 | 1.15E+08 | 1.15E+08 | 0.033 | Tbx3 | 0.039599 | 0.080103 | 0 | −2877534 |
| chr12 | 1.15E+08 | 1.15E+08 | 0.017 | Tbx3 | 0.002757 | 0.001537 | −2373 | −2894447 |
| chr12 | 1.15E+08 | 1.15E+08 | 0.005 | Tbx3 | 0.000306 | 0.001286 | −5650 | −2897724 |
| chr12 | 1.19E+08 | 1.19E+08 | 0.012 | Taok3 | 0.030025 | 0.024758 | 0 | −293657 |
| chr22 | 40858582 | 40860665 | 0.005 | Mkl1 | 0.001385 | 0.019491 | −17872 | −3262460 |
| chr17 | 1220102 | 1220359 | 0.005 | Tusc5 | 0.004981 | 0.331278 | 0 | 0 |
| chr17 | 7483300 | 7483645 | 0.029 | Cd68 | 0.004383 | 0.004552 | 0 | 252958 |
| chr17 | 8027102 | 8027420 | 0.029 | Hes7 | 0.039742 | 0.158273 | 2176 | −277411 |
| chr17 | 34415897 | 34416118 | 0.026 | Ccl3 | 0.001156 | 0.04389 | −114527 | 1419299 |
| chr17 | 34417557 | 34418034 | 0.009 | Ccl3 | 0.001156 | 0.04389 | −116187 | 1417383 |
| chr17 | 47819090 | 47819795 | 0.005 | 5730593F17Rik | 0.0074 | 0.007408 | 0 | −1152018 |
| chr17 | 56355449 | 56355594 | 0.044 | Mpo | 0.038596 | 0.084256 | 57556 | 1050065 |
| chr17 | 79419543 | 79420749 | 0.006 | Bahcc1 | 0.017499 | 0.024719 | 13038 | −15458 |
| chr17 | 80052124 | 80053059 | 0.009 | Fasn | 0.002345 | 0.045378 | 862 | −648039 |
| chr5 | 34655547 | 34655935 | 0.029 | Rai14 | 0.044333 | 0.057769 | 301318 | −2479732 |
| chr5 | 34687673 | 34687965 | 0.029 | Rai14 | 0.027203 | 0.000317 | 269288 | −2511858 |
| chr5 | 66491448 | 66492361 | 0.005 | Cd180 | 0.010515 | 0.032888 | −981 | −161032 |
| chr5 | 67585536 | 67585875 | 0.033 | Pik3r1 | 0.027604 | 0.050059 | 0 | 0 |
| chr5 | 77803248 | 77803367 | 0.03 | Lhfpl2 | 0.014339 | 0.000121 | 0 | 0 |
| chr5 | 1.07E+08 | 1.07E+08 | 0.011 | Efna5 | 0.04026 | 0.005299 | 0 | 8368099 |
| chr5 | 1.08E+08 | 1.08E+08 | 0.011 | Fert2 | 0.01459 | 0.032578 | 0 | 7190713 |
| chr5 | 1.48E+08 | 1.48E+08 | 0.006 | Adrb2 | 0.005235 | 0.055308 | 0 | 304318 |
| chr5 | 1.49E+08 | 1.49E+08 | 0.006 | Csflr | 0.0083 | 0.011174 | 25190 | 398535 |
| chr5 | 1.7E+08 | 1.7E+08 | 0.011 | Kcnmb1 | 1.42E−05 | 0.006693 | −564 | 2397729 |
| chr5 | 1.79E+08 | 1.79E+08 | 0.012 | Adamts2 | 0.010601 | 0.004506 | −49139 | −49139 |
| chr7 | 1408433 | 1408951 | 0.006 | Micall2 | 0.010304 | 0.002223 | 89119 | 5005012 |
| chr7 | 26460385 | 26460633 | 0.015 | Snx10 | 0.017227 | 0.02051 | −20840 | −217203 |
| chr7 | 27201206 | 27201499 | 0.027 | Hoxa9 | 0.000244 | 0.000896 | 0 | 0 |
| chr7 | 27202553 | 27202993 | 0.044 | Hoxa9 | 0.007726 | 0.098969 | 0 | 0 |
| chr7 | 27203073 | 27203466 | 0.008 | Hoxa9 | 0.007726 | 0.098969 | 0 | 0 |
| chr7 | 50345715 | 50346835 | 0.005 | Ikzf1 | 0.000204 | 0.000649 | −235589 | −2784315 |
| chr7 | 73624879 | 73625346 | 0.005 | Lat2 | 0.025835 | 0.073432 | −14413 | 415807 |
| chr7 | 76828348 | 76828576 | 0.042 | Fgl2 | 0.032838 | 0.072158 | 16610 | −570377 |
| chr7 | 1.07E+08 | 1.07E+08 | 0.005 | Prkar2b | 0.001245 | 0.052519 | 0 | 0 |
| chr7 | 1.4E+08 | 1.4E+08 | 0.005 | Tbxas1 | 7.05E−05 | 0.012069 | −51336 | −74443 |
| chr14 | 75361059 | 75361571 | 0.033 | Dlst | 0.002412 | 0.006712 | −1825 | −1098230 |
| chr14 | 77791405 | 77791671 | 0.011 | Gstz1 | 0.010969 | 0.049885 | −8940 | −265105 |
| chr14 | 89707672 | 89708616 | 0.009 | Foxn3 | 0.005944 | 0.097831 | −13657 | 1138563 |
| chr14 | 93117257 | 93117768 | 0.029 | Rin3 | 0.003787 | 0.012258 | −63158 | 411618 |
| chr14 | 1.01E+08 | 1.01E+08 | 0.006 | Evl | 0.003391 | 0.00473 | 9386 | 2104521 |
| chr9 | 14203697 | 14204081 | 0.005 | Nfib | 0.001304 | 0.009562 | 0 | 0 |
| chr9 | 32457089 | 32457364 | 0.005 | Ddx58 | 0.019333 | 0.303952 | −76 | −76 |
| chr9 | 95726604 | 95726801 | 0.03 | Fgd3 | 0.000783 | 0.106858 | −25224 | −1533887 |
| chr9 | 1.14E+08 | 1.14E+08 | 0.006 | Gng10 | 0.008616 | 0.004046 | 0 | −1506896 |
| chr9 | 1.16E+08 | 1.16E+08 | 0.033 | Rgs3 | 0.034041 | 0.07686 | 0 | 0 |
| chr9 | 1.18E+08 | 1.18E+08 | 0.016 | Tnfsf8 | 0.000934 | 0.010527 | 0 | −531146 |
| chr9 | 1.31E+08 | 1.31E+08 | 0.005 | Spna2 | 0.021423 | 0.017082 | 0 | −503909 |
| chr13 | 36049213 | 36050887 | 0.005 | Mab2111 | 0.000513 | 0.000333 | 0 | −2183260 |
| chr13 | 40943529 | 40943850 | 0.006 | Foxo1 | 0.045248 | 0.09776 | 0 | 175911 |
| chr13 | 46751279 | 46752608 | 0.005 | Lcp1 | 0.014544 | 0.021646 | −5166 | 2041932 |
| chr13 | 46753288 | 46754147 | 0.009 | Lcp1 | 0.014544 | 0.021646 | −7175 | 2040393 |
| chr13 | 50204794 | 50205145 | 0.022 | Arl11 | 0.04428 | 0.046926 | −4181 | −1375720 |
| chr3 | 9911613 | 9911971 | 0.029 | Cidec | 0.014016 | 0.028881 | 0 | 0 |
| chr3 | 24869934 | 24870287 | 0.008 | Rarb | 0.029778 | 0.111071 | −186244 | 4450134 |
| chr3 | 1.13E+08 | 1.13E+08 | 0.005 | Cd200r1 | 0.009567 | 0.011117 | 42134 | −318068 |
| chr3 | 1.13E+08 | 1.13E+08 | 0.008 | Boc | 0.026238 | 0.048245 | 339 | 488239 |
| chr3 | 1.5E+08 | 1.5E+08 | 0.012 | 2810407C02Rik | 0.000504 | 0.000438 | 0 | −184142 |
| chr3 | 1.57E+08 | 1.57E+08 | 0.006 | Veph1 | 0.000152 | 0.004779 | −33016 | −430940 |
| chr16 | 16123803 | 16124667 | 0.005 | Abcc1 | 0.028138 | 0.092617 | 0 | −1689502 |
| chr16 | 50715116 | 50715299 | 0.011 | 9130017C17Rik | 0.000205 | 0.012381 | 13526 | −2286067 |
| chr16 | 55540360 | 55540833 | 0.006 | Mmp2 | 0.012314 | 0.083101 | −17985 | −2364486 |
| chr16 | 56137894 | 56138256 | 0.006 | Gnao1 | 0.004769 | 0.003215 | −266909 | −2962020 |
| chr16 | 69457347 | 69457490 | 0.044 | Cyb5b | 0.012444 | 0.184651 | −5180 | −1121558 |
| chr16 | 87903345 | 87904117 | 0.006 | BC048644 | 0.034324 | 0.034225 | 47037 | −2293210 |
| chr16 | 88770501 | 88770848 | 0.012 | Rnf166 | 0.04324 | 0.137684 | 0 | −3160366 |
| chr21 | 36398548 | 36398828 | 0.011 | Runx1 | 0.000152 | 0.001644 | 20276 | −134293 |
| chr21 | 36420456 | 36421805 | 0.005 | Runx1 | 4.31E−06 | 0.00056 | 0 | −156201 |
| chr21 | 39868683 | 39869555 | 0.01 | Erg | 0.000108 | 0.004244 | 0 | 0 |
| chr18 | 56809110 | 56809370 | 0.017 | Sec11c | 0.047576 | 0.081715 | −86050 | −3789034 |
| chr19 | 15574480 | 15574831 | 0.046 | A430107D22Rik | 0.019917 | 0.084596 | −10634 | −153351 |
| chr19 | 40750567 | 40750963 | 0.015 | Akt2 | 0.005362 | 0.026742 | 0 | 0 |
| chr19 | 49841249 | 49841895 | 0.009 | Cd37 | 0.027967 | 0.000388 | 493 | −1591657 |
| chr19 | 51645546 | 51714605 | 0.006 | Cd33 | 0.026485 | 0.042378 | −3881 | −3395954 |
| chr19 | 54876429 | 54876765 | 0.03 | Lair1 | 0.02649 | 0.000733 | 0 | −6626837 |
| chr11 | 6267881 | 6268720 | 0.012 | Cnga4 | 0.001427 | 0.095175 | −19648 | 354360 |
| chr11 | 10316893 | 10317243 | 0.035 | Adm | 0.015286 | 0.074089 | 0 | 0 |
| chr11 | 10324978 | 10325218 | 0.033 | Adm | 0.006833 | 0.022077 | 0 | 0 |
| chr11 | 47415025 | 47415403 | 0.006 | Sfpi1 | 0.005613 | 0.020093 | 0 | 100118 |
| chr11 | 63528336 | 63528808 | 0.006 | 2700081O15Rik | 0.009375 | 0.019784 | 5253 | −36600 |
| chr11 | 66673568 | 66673798 | 0.043 | Pcx | 0.049345 | 0.126683 | 0 | 0 |
| chr11 | 67036383 | 67036711 | 0.029 | Adrbk1 | 0.000862 | 0.093207 | 0 | −332714 |
| chr11 | 67165584 | 67166226 | 0.009 | Ppp1ca | 0.038535 | 0.003821 | −23002 | −461915 |
| chr11 | 73692113 | 73692198 | 0.028 | Ucp2 | 0.048033 | 0.079853 | −2531 | 1319884 |
| chr11 | 1.18E+08 | 1.18E+08 | 0.022 | Il10ra | 0.000608 | 0.020984 | 0 | 613105 |
| chr11 | 1.23E+08 | 1.23E+08 | 0.015 | Bsx | 0.034711 | 0.01019 | −44045 | 78806 |
| chr15 | 37173455 | 37174113 | 0.026 | Meis2 | 0.028601 | 0.011445 | 210804 | 2244812 |
| chr15 | 61136559 | 61136807 | 0.024 | Rora | 0.0012 | 0.091815 | −42316 | −416528 |
| chr15 | 96902600 | 96903113 | 0.005 | Nr2f2 | 4.02E−05 | 0.001088 | −1250 | −1250 |
| chr15 | 96905568 | 96906197 | 0.006 | Nr2f2 | 0.000917 | 7.85E−06 | −4218 | −4218 |
| chr15 | 96907528 | 96907914 | 0.022 | Nr2f2 | 0.000917 | 7.85E−06 | −6178 | −6178 |
| chr20 | 18269464 | 18269677 | 0.029 | 6330439K17Rik | 0.004138 | 0.002225 | 31641 | 1674677 |
| chr20 | 25033629 | 25034262 | 0.005 | Acss1 | 0.035246 | 0.043383 | 0 | −5041490 |
| chr20 | 25251494 | 25252151 | 0.005 | Pygb | 0.009127 | 0.014408 | 831 | 5027244 |
| chr20 | 39314354 | 39315324 | 0.006 | Mafb | 0.016336 | 0.014954 | 0 | 3884911 |
| chr20 | 47803610 | 47804113 | 0.006 | Stau1 | 0.048578 | 0.316221 | 0 | 485791 |
| chr20 | 48924232 | 48924768 | 0.022 | Cebpb | 0.004243 | 0.078455 | 0 | 0 |
| chr20 | 56135219 | 56135721 | 0.006 | Pck1 | 0.008352 | 0.046531 | 0 | 122179 |
| chr20 | 56135935 | 56137320 | 0.005 | Pck1 | 0.003188 | 0.040125 | 0 | 120580 |
| chr20 | 56138648 | 56138937 | 0.016 | Pck1 | 0.003188 | 0.040125 | 0 | 118963 |
| chr20 | 56139464 | 56139982 | 0.009 | Pck1 | 0.003188 | 0.040125 | 0 | 117918 |
| chr4 | 38858508 | 38859749 | 0.008 | Tlr6 | 0.003242 | 0.050924 | 0 | −157838 |
| chr4 | 84034389 | 84034401 | 0.015 | Plac8 | 0.000416 | 0.009649 | −99147 | 3885033 |
| chr4 | 1.09E+08 | 1.09E+08 | 0.024 | Hadh | 0.044534 | 0.171435 | 0 | 0 |
| chr4 | 1.14E+08 | 1.14E+08 | 0.005 | Larp7 | 0.027309 | 0.053868 | −15696 | 491614 |
| chr4 | 1.84E+08 | 1.84E+08 | 0.006 | Cldn22 | 0.047192 | 0.009106 | 75490 | 1424121 |
| chr4 | 1.85E+08 | 1.85E+08 | 0.005 | Stox2 | 0.010584 | 0.081507 | −21381 | 890761 |
| chr4 | 1.85E+08 | 1.85E+08 | 0.006 | Irf2 | 0.04424 | 0.017467 | 0 | 361837 |

This table displays the 171 cross-species conserved and directionally consistent regions with differential methylation along with the nearest enhancer and super enhancer found in adipose tissue (see Methods).

TABLE 11
Human Subject Information, related to Experimental Procedures.

| Patient | | | | | Waist | pre-op | pre-op | pre-op | Preop | pre-op |
|---|---|---|---|---|---|---|---|---|---|---|
| Number | | Type 2 | Age at | | circumference | HDL | LDL | Cholesterol | HbA1C | HbA1c |
| All Male | Obesity | Diabetes | surgery | BMI | (cm) | (mol/L) | (mol/L) | (mol/L) | mmol/mol | (%) |
| 1 | Ob | T2D | 42 | 37.00 | 118 | 1 | 1.3 | 2.9 | 48 | 5.6 |
| 2 | Ob | T2D | 62 | 36.00 | 143 | 0.6 | 2 | 3.6 | 43 | 5.2 |
| 3 | Ob | ND | 43 | 40.30 | 137 | 2 | 3.4 | 5.8 | 39 | 4.8 |
| 4 | Ob | T2D | 56 | 37.10 | 124 | 0.9 | | 4 | 71 | 7.8 |
| 5 | Ob | ND | 36 | 41.60 | 125 | 1.1 | 3.7 | 5.5 | 34 | 4.3 |
| 6 | Ob | T2D | 52 | 42.90 | 140 | 0.9 | 3.7 | 5.2 | 52 | 6 |
| 7 | Ob | ND | 49 | 40.00 | 140 | 1.4 | 3.8 | 6.1 | 40 | 4.9 |
| 8 | Ob | T2D | 41 | 40.40 | 125 | 3.9 | 3.7 | 5 | 69 | 7.6 |
| 9 | Ob | ND | 50 | 35.60 | 122 | 0.4 | 2.3 | 3.9 | 41 | 5 |
| 10 | Ob | T2D | 34 | 40.30 | 139 | 1 | 4.9 | 6.9 | 48 | 5.6 |
| 11 | Ob | T2D | 37 | 42.70 | 139 | 0.7 | 1.7 | 3.6 | 49 | 5.7 |
| 12 | Ob | T2D | 36 | 45.84 | 143 | 0.6 | 3.6 | 4.9 | | 5.6 |
| 13 | Ob | ND | 41 | 37.22 | 122 | 1.1 | 4.4 | 6.2 | 4.4 | 5.8 |
| 14 | Ob | ND | 59 | 37.24 | 120 | 1 | 3.1 | 4.9 | | 4.2 |
| 15 | L | ND | 39 | 24.50 | 87 | 1.1 | 3.9 | 6 | 38 | 4.7 |
| 16 | L | ND | 41 | 27.80 | 89 | 0.9 | 3.1 | 4.6 | 37 | 4.6 |
| 17 | L | ND | 59 | 25.10 | 100 | 1.3 | 4.7 | 6.5 | 36 | 4.5 |
| 18 | L | ND | 36 | 26.30 | 99 | 1.4 | 3.6 | 5.3 | 41 | 5 |
| 19 | L | ND | 39 | 28.40 | 102 | 1 | 3 | 4.6 | 37 | 4.6 |
| 20 | L | ND | 49 | 22.60 | 80 | 1.5 | 3.7 | 5.7 | 38 | 4.7 |
| 21 | L | ND | 41 | 20.00 | | | | | | |

TABLE 12
Human Subject Information, related to Experimental Procedures.

| | | | | | | insulin 2 | HOMA- | | | | |
|---|---|---|---|---|---|---|---|---|---|---|---|
| Patient | | | | | | days | IR 2 | | | | |
| Number | pre-op | insulin | insulin | HOMA- | preop | after | days | follow | Weight | BMI | Waist |
| All | glucose | pre-op | pre-op | IR pre- | TG | RYGB | after | up time | follow-up | follow- | followup |
| Male | (mmol/L) | (pg/ml) | (pmol/L) | op | (mol/L) | (pmol/L) | RYGB | (month) | (kg) | up | (cm) |
| 1 | 6.7 | | | | 1.2 | | | | | | |
| 2 | 6.7 | 1139.035 | 196.1148 | 45.32432 | 3.3 | 118.6123 | 1033.85 | 8 | 128.1 | 31.4 | 129 |
| 3 | 5.5 | 721.27 | 124.1856 | 26.49293 | 0.9 | 140.6043 | 776.046 | 7 | 112.5 | 31.5 | |
| 4 | 17.3 | 893.445 | 153.8301 | 53.32775 | 6.1 | 114.697 | 784.1708 | 7 | 87.9 | 28.4 | 100 |
| 5 | 5.4 | 1068.2 | 183.9187 | 35.14891 | 1.6 | | | | | | |
| 6 | 7.5 | 973.01 | 167.5293 | 44.67447 | 1.2 | 182.0532 | 1355.522 | 6 | 115.2 | 34.8 | 119 |
| 7 | 5.9 | 579.32 | 99.74518 | 21.72228 | 2 | 100.1154 | 443.8233 | 5 | 112 | 32 | 112 |
| 8 | 8.2 | 850.8 | 146.4876 | 49.48026 | 1.4 | 131.0348 | 853.1098 | 7 | 98.8 | 31.9 | 118 |
| 9 | 5.3 | 893.095 | 153.7698 | 34.17107 | 2.6 | | | | | | |
| 10 | 7.9 | 1066.505 | 183.6269 | 45.70269 | 2.2 | 227.0377 | 1852.899 | 9 | 96.2 | 30 | 108 |
| 11 | 8.2 | 1347.56 | 232.0179 | 58.77787 | 2.5 | 178.3385 | 1839.01 | 5 | 127.8 | 35.4 | 122 |
| 12 | 6.6 | 1740.36 | 299.6488 | 74.57925 | 1.5 | 177.1935 | 2359.814 | | | | |
| 13 | | | | | | | | | | | |
| 14 | 5.2 | 1160.125 | 199.746 | 37.28593 | 1.9 | 241.5694 | 2144.557 | | | | |
| 15 | 5.9 | 87.675 | 40 | 8.4 | 2.2 | | | | | | |
| 16 | 5.7 | 561.845 | 189 | 38.6 | 1.3 | | | | | | |
| 17 | 4.8 | 261.13 | 62.3 | 12.5 | 1 | | | | | | |
| 18 | 5.3 | 375.42 | 64.6 | 14.4 | 0.92 | | | | | | |
| 19 | 5.5 | 363.32 | 62.6 | 12.8 | 1.3 | | | | | | |
| 20 | 5.9 | 240.46 | 41.4 | 8.6 | 1 | | | | | | |
| 21 | | | 47.2 | | | | | | | | |

This table displays relevant information about the human subjects examined in this study.

Although the invention has been described with reference to the examples herein, it will be understood that modifications and variations are encompassed within the spirit and scope of the invention. Accordingly, the invention is limited only by the following claims.

What is claimed is:

1. A method for identifying a subject having or at risk of having a metabolic disease comprising identifying in the subject one or more genetic markers correlating differentially methylated regions (DMRs) in the genome with genetic risk loci for the subject and comparing methylation patterns of the markers with a control sample from a subject not having the disease, thereby identifying the subject as having or at risk of having a metabolic disease.

2. The method of claim 1, wherein the disease is diabetes or obesity.

3. The method of claim 2, wherein the disease is diabetes.

4. The method of claim 3, wherein the disease is type 2 diabetes (T2D).

5. The method of claim 1, wherein the genetic markers are hypermethylated or hypomethylated.

6. The method of claim 1, wherein the genetic markers are selected from 2 or more genes as set forth in Table 2.

7. The method of claim 4, wherein the genetic markers include at least Tcf712.

8. The method of claim 4, wherein the genetic markers are selected from Mkl1, Plekho1, Tnfaip812, Tcf712, Prc1, Foxo1, Plekho1, Fasn, App, Akt2, or any combination thereof.

9. The method of claim 8, wherein the genetic markers are Mkl1, Plekho1 and Tnfaip812.

10. The method of claim 9, wherein the genetic markers are hypomethylated.

11. The method of claim 1, further comprising analyzing adipose cells of the subject, wherein an inflammatory response is a factor associated with having or risk of having T2D.

12. The method of claim 1, wherein identifying comprises determining methylation status of genetic markers.

13. The method of claim 12, wherein the methylation status is performed by one or more techniques selected from the group consisting of a nucleic acid amplification, polymerase chain reaction (PCR), methylation specific PCR, bisulfite pyrosequencing, single-strand conformation polymorphism (SSCP) analysis, restriction analysis, microarray technology, and proteomics.

14. The method of claim 1, wherein the genetic markers are identified from a sample from the subject, wherein the sample is selected from blood, adipose tissue, pancreatic tissue, liver tissue, serum, urine, saliva, cerebrospinal fluid, pleural fluid, ascites fluid, sputum, and stool.

15. A method of treating a subject having or at risk of having a metabolic disease comprising increasing or decreasing gene expression of one or more genetic markers correlated with genetic risk loci for the subject based on an observation of hypomethylation or hypermethylation, respectively, of the marker, thereby treating the subject.

16. The method of claim 15, wherein the genetic markers affect glucose utilization by a cell.

17. The method of claim 15, wherein the genetic markers are associated with obesity.

18. The method of claim 15, wherein the genetic markers are associated with diabetes.

19. The method of claim 18, wherein the diabetes is type 2 diabetes (T2D).

20. The method of claim 15, wherein the genetic markers are selected from 2 or more genes as set forth in Table 2.

21. The method of claim 15, wherein the genetic markers include at least Tcf712.

22. The method of claim 15, wherein the genetic marker are selected from Mkl1, Plekho1, Tnfaip812, Tcf712, Prc1, Foxo1, Plekho1, Fasn, App, Akt2, or any combination thereof.

23. The method of claim 22, wherein the genetic markers are Mkl1, Plekho1 and Tnfaip812.

24. The method of claim 23, wherein the genetic markers are hypomethylated.

25. The method of claim 15, wherein the genetic markers are identified from a sample from the subject, wherein the sample is selected from blood, adipose tissue, pancreatic tissue, liver tissue, serum, urine, saliva, cerebrospinal fluid, pleural fluid, ascites fluid, sputum, and stool.

26. A method of providing a prognostic evaluation of a subject having or at risk of having a metabolic disease comprising analyzing one or more genetic markers of the subject which is correlated with genetic risk loci prior to dietary and/or pharmaceutical intervention and following dietary and/or pharmaceutical intervention, and correlating a change in the genetic markers with a prognostic evaluation of the subject, thereby providing a prognostic evaluation.

27. The method of claim 26, wherein a decrease in expression of a marker previously up-regulated is correlated with improvement in the metabolic disorder.

28. The method of claim 26, wherein an increase in expression of a marker previously down-regulated is correlated with improvement in the metabolic disorder.

29. The method of claim 26, wherein the disease is diabetes or obesity.

30. The method of claim 29, wherein the disease is diabetes.

31. The method of claim 30, wherein the disease is type 2 diabetes (T2D).

32. The method of claim 26, wherein the genetic markers are hypermethylated or hypomethylated.

33. The method of claim 26, wherein the genetic markers are selected from 2 or more genes as set forth in Table 2.

34. The method of claim 33, wherein the genetic markers include at least Tcf712.

35. The method of claim 33, wherein the genetic markers are selected from Mkl1, Plekho1, Tnfaip812, Tcf712, Prc1, Foxo1, Plekho1, Fasn, App, Akt2, or any combination thereof.

36. The method of claim 35, wherein the genetic markers are Mkl1, Plekho1 and Tnfaip812.

37. The method of claim 36, wherein the genetic markers are hypomethylated.

38. The method of claim 26, wherein the genetic markers are identified from a sample from the subject, wherein the sample is selected from blood, adipose tissue, pancreatic tissue, liver tissue, serum, urine, saliva, cerebrospinal fluid, pleural fluid, ascites fluid, sputum, and stool.

39. A method for identifying a subject having or at risk of having a metabolic disease, cancer, immune system disorder, cardiovascular disease, gastrointestinal disease or pulmonary disease comprising identifying in the subject genetic markers correlating differentially methylated regions (DMRs) in the genome with genetic risk loci for the subject and comparing methylation patterns of the markers with a control sample from a subject not having the disease.

40. The method of claim 39, wherein the metabolic disease is diabetes or obesity.

41. The method of claim 20, wherein the metabolic disease is diabetes.

42. The method of claim 41, wherein the metabolic disease is type 2 diabetes (T2D).

43. The method of claim 39, wherein the genetic markers are hypermethylated or hypomethylated.

44. The method of claim 39, wherein the genetic markers are selected from 2 or more genes as set forth in Table 2.

45. The method of claim 44, wherein the genetic markers include at least Tcf712.

46. The method of claim 44, wherein the genetic markers are selected from Mkl1, Plekho1, Tnfaip812, Tcf712, Prc1, Foxo1, Plekho1, Fasn, App, Akt2, or any combination thereof.

47. The method of claim 46, wherein the genetic markers are Mkl1, Plekho1 and Tnfaip812.

48. The method of claim 47, wherein the genetic markers are hypomethylated.

49. A method of determining a therapeutic regimen for a subject comprising identifying in the subject genetic markers correlating differentially methylated regions (DMRs) in the genome with genetic risk loci for the subject and comparing methylation patterns of the markers with a control sample from a subject thereby assessing the therapeutic regimen for the subject.

50. The method of claim 49, wherein the subject has, or is at risk of having a metabolic disease.

51. The method of claim 50, wherein the metabolic disease is diabetes or obesity.

52. The method of claim 51, wherein the metabolic disease is diabetes.

53. The method of claim 52, wherein the metabolic disease is type 2 diabetes (T2D).

54. The method of claim 49, wherein the genetic markers are hypermethylated or hypomethylated.

55. The method of claim 49, wherein the genetic markers are selected from 2 or more genes as set forth in Table 2.

56. The method of claim 55, wherein the genetic markers include at least Tcf712.

57. The method of claim 55, wherein the genetic markers are selected from Mkl1, Plekhol, Tnfaip812, Tcf712, Prc1, Foxo1, Plekho1, Fasn, App, Akt2, or any combination thereof.

58. The method of claim 57, wherein the genetic markers are Mkl1, Plekho1 and Tnfaip812.

59. The method of claim 58, wherein the genetic markers are hypomethylated.


SEQUENCE LISTING (155 sequences)

SEQ ID NO: 1 - DNA, 27 bp, Artificial Sequence
  Pyrosquencing Primer
ttgagtttgttaaatttaggggtaagt

SEQ ID NO: 2 - DNA, 26 bp, Artificial Sequence
  Pyrosquencing Primer
ttcaaacaacatttttaaatcatttc

SEQ ID NO: 3 - DNA, 27 bp, Artificial Sequence
  Pyrosquencing Primer
ttgagtttgttaaatttaggggtaagt

SEQ ID NO: 4 - DNA, 29 bp, Artificial Sequence
  Pyrosquencing Primer
caaacaaaaactattaactctaaaaccac

SEQ ID NO: 5 - DNA, 18 bp, Artificial Sequence
  Pyrosquencing Primer
gttattagagtaagtgta

SEQ ID NO: 6 - DNA, 20 bp, Artificial Sequence
  Pyrosquencing Primer
gataaaatttaaagagtgtt

SEQ ID NO: 7 - DNA, 22 bp, Artificial Sequence
  Pyrosquencing Primer
gtttaagtatttattaaaatat

SEQ ID NO: 8 - DNA, 25 bp, Artificial Sequence
  Pyrosquencing Primer
ttttttagggaaaagaatttttttt

SEQ ID NO: 9 - DNA, 25 bp, Artificial Sequence
  Pyrosquencing Primer
atctcaaaccacaacaatcacataa

SEQ ID NO: 10 - DNA, 29 bp, Artificial Sequence
  Pyrosquencing Primer
ggtattatttttagtagaggttaggtaaa

SEQ ID NO: 11 - DNA, 25 bp, Artificial Sequence
  Pyrosquencing Primer
caaaaccaaaaaaccatatattaac

SEQ ID NO: 12 - DNA, 27 bp, Artificial Sequence
  Pyrosquencing Primer
ggtattatttttagtagaggttaggta

SEQ ID NO: 13 - DNA, 23 bp, Artificial Sequence
  Pyrosquencing Primer
atagtgtttgttagtatttgaat

SEQ ID NO: 14 - DNA, 27 bp, Artificial Sequence
  Pyrosquencing Primer
ttttataataattgaaggagggaaagt

SEQ ID NO: 15 - DNA, 26 bp, Artificial Sequence
  Pyrosquencing Primer
aaaaacaatataacccaaacaaaaac

SEQ ID NO: 16 - DNA, 27 bp, Artificial Sequence
  Pyrosquencing Primer
ttttataataattgaaggagggaaagt

SEQ ID NO: 17 - DNA, 25 bp, Artificial Sequence
  Pyrosquencing Primer
tacacacttacctaataatcaaacc

SEQ ID NO: 18 - DNA, 25 bp, Artificial Sequence
  Pyrosquencing Primer
gaatagttgtttttttttttaatat

SEQ ID NO: 19 - DNA, 21 bp, Artificial Sequence
  Pyrosquencing Primer
aattttaaagtaaaggttatg

SEQ ID NO: 20 - DNA, 26 bp, Artificial Sequence
  Pyrosquencing Primer
tggtaaaggttttgttgtttaagtgt

SEQ ID NO: 21 - DNA, 25 bp, Artificial Sequence
  Pyrosquencing Primer
attctctaaccatcccaaaataaac

SEQ ID NO: 22 - DNA, 25 bp, Artificial Sequence
  Pyrosquencing Primer
tttttagatatttgggtatttaaga

SEQ ID NO: 23 - DNA, 30 bp, Artificial Sequence
  Pyrosquencing Primer
actataaactttattctaacaaaacaatac

SEQ ID NO: 24 - DNA, 20 bp, Artificial Sequence
  Pyrosquencing Primer
gtttgatgtatatttttttg

SEQ ID NO: 25 - DNA, 20 bp, Artificial Sequence
  Pyrosquencing Primer
tttagagtaggggttagtat

SEQ ID NO: 26 - DNA, 25 bp, Artificial Sequence
  Pyrosquencing Primer
agttgtataatttattgtgggttgg

SEQ ID NO: 27 - DNA, 25 bp, Artificial Sequence
  Pyrosquencing Primer
tcctttaaaacttcctcaaacattt

SEQ ID NO: 28 - DNA, 25 bp, Artificial Sequence
  Pyrosquencing Primer
agttatggagggttaatttgtgttt

SEQ ID NO: 29 - DNA, 25 bp, Artificial Sequence
  Pyrosquencing Primer
tcctttaaaacttcctcaaacattt

SEQ ID NO: 30 - DNA, 20 bp, Artificial Sequence
  Pyrosquencing Primer
gttaatttgtgtttgggatt

SEQ ID NO: 31 - DNA, 22 bp, Artificial Sequence
  Pyrosquencing Primer
atttttagaggattgggtgaag

SEQ ID NO: 32 - DNA, 18 bp, Artificial Sequence
  Pyrosquencing Primer
ggagttattgataaattt

SEQ ID NO: 33 - DNA, 25 bp, Artificial Sequence
  Pyrosquencing Primer
agagtttaggttataggtgggaggt

SEQ ID NO: 34 - DNA, 25 bp, Artificial Sequence
  Pyrosquencing Primer
aaaatatcctaccaaccaaaaaaaa

SEQ ID NO: 35 - DNA, 25 bp, Artificial Sequence
  Pyrosquencing Primer
tttaggttatgtttaagggttttgg

SEQ ID NO: 36 - DNA, 25 bp, Artificial Sequence
  Pyrosquencing Primer
aaaactaaaaaacccacacaaacac

SEQ ID NO: 37 - DNA, 27 bp, Artificial Sequence
  Pyrosquencing Primer
atttttttttttagtaatttagataag

SEQ ID NO: 38 - DNA, 21 bp, Artificial Sequence
  Pyrosquencing Primer
attttattttttagtttttgg

SEQ ID NO: 39 - DNA, 21 bp, Artificial Sequence
  Pyrosquencing Primer
gaggtgagtttaggtagattt

SEQ ID NO: 40 - DNA, 25 bp, Artificial Sequence
  Pyrosquencing Primer
ttttgagggtttagtggtagtttgt

SEQ ID NO: 41 - DNA, 29 bp, Artificial Sequence
  Pyrosquencing Primer
caaattcaataaaaacacaaattaaaaaa

SEQ ID NO: 42 - DNA, 28 bp, Artificial Sequence
  Pyrosquencing Primer
agggtttagtggtagtttgttaggttat

SEQ ID NO: 43 - DNA, 30 bp, Artificial Sequence
  Pyrosquencing Primer
aaaaaaaatcaacaaaaacaatattaaatt

SEQ ID NO: 44 - DNA, 20 bp, Artificial Sequence
  Pyrosquencing Primer
aaattgtttattttttgtag

SEQ ID NO: 45 - DNA, 23 bp, Artificial Sequence
  Pyrosquencing Primer
ttttttattttattttatttttt

SEQ ID NO: 46 - DNA, 26 bp, Artificial Sequence
  Pyrosquencing Primer
ggagtttttattaggagagggtgtag

SEQ ID NO: 47 - DNA, 25 bp, Artificial Sequence
  Pyrosquencing Primer
ataaaccaaaataacctccaactca

SEQ ID NO: 48 - DNA, 27 bp, Artificial Sequence
  Pyrosquencing Primer
tattttgatggtgttagagatgaattt

SEQ ID NO: 49 - DNA, 22 bp, Artificial Sequence
  Pyrosquencing Primer
ccaccaccacctaaaaaaaact

SEQ ID NO: 50 - DNA, 19 bp, Artificial Sequence
  Pyrosquencing Primer
atggtgttagagatgaatt

SEQ ID NO: 51 - DNA, 20 bp, Artificial Sequence
  Pyrosquencing Primer
gtatagttatatttttaaat

SEQ ID NO: 52 - DNA, 22 bp, Artificial Sequence
  Pyrosquencing Primer
ttatttgatgtttaatatattt

SEQ ID NO: 53 - DNA, 25 bp, Artificial Sequence
  Pyrosquencing Primer
gggagtttagaatagtgattggttg

SEQ ID NO: 54 - DNA, 25 bp, Artificial Sequence
  Pyrosquencing Primer
tcccttccttaaataaccttcctac

SEQ ID NO: 55 - DNA, 27 bp, Artificial Sequence
  Pyrosquencing Primer
gggaagatttttaaatagagaggatgt

SEQ ID NO: 56 - DNA, 25 bp, Artificial Sequence
  Pyrosquencing Primer
taccctaaaaactaaaccccaaaac

SEQ ID NO: 57 - DNA, 23 bp, Artificial Sequence
  Pyrosquencing Primer
gatatttaatataatatttattt

SEQ ID NO: 58 - DNA, 22 bp, Artificial Sequence
  Pyrosquencing Primer
attgagttttaggttagtttta

SEQ ID NO: 59 - DNA, 20 bp, Artificial Sequence
  Pyrosquencing Primer
ttgggggaattaatttaagt

SEQ ID NO: 60 - DNA, 25 bp, Artificial Sequence
  Pyrosquencing Primer
atagtttaggaggaaagttggtgtt

SEQ ID NO: 61 - DNA, 25 bp, Artificial Sequence
  Pyrosquencing Primer
aaaaacttccaaaacataaaaaaaa

SEQ ID NO: 62 - DNA, 26 bp, Artificial Sequence
  Pyrosquencing Primer
aagggatttaagggatttttaaattt

SEQ ID NO: 63 - DNA, 24 bp, Artificial Sequence
  Pyrosquencing Primer
caactccctacaacaaaacacttt

SEQ ID NO: 64 - DNA, 17 bp, Artificial Sequence
  Pyrosquencing Primer
ggtagtttggtttattt

SEQ ID NO: 65 - DNA, 18 bp, Artificial Sequence
  Pyrosquencing Primer
gattatttagatgtttgt

SEQ ID NO: 66 - DNA, 19 bp, Artificial Sequence
  Pyrosquencing Primer
gtggtatgataggtagtag

SEQ ID NO: 67 - DNA, 25 bp, Artificial Sequence
  Pyrosquencing Primer
gggttttaagaggttgttggttaat

SEQ ID NO: 68 - DNA, 25 bp, Artificial Sequence
  Pyrosquencing Primer
aaccctaaacaaaacacaatttcac

SEQ ID NO: 69 - DNA, 24 bp, Artificial Sequence
  Pyrosquencing Primer
tagtttgttttgggataggttgtg

SEQ ID NO: 70 - DNA, 25 bp, Artificial Sequence
  Pyrosquencing Primer
aatatacccccaaaaataaaaaacc

SEQ ID NO: 71 - DNA, 22 bp, Artificial Sequence
  Pyrosquencing Primer
gaatataaaggttaagtgttta

SEQ ID NO: 72 - DNA, 21 bp, Artificial Sequence
  Pyrosquencing Primer
agagtttgggtagttagatag

SEQ ID NO: 73 - DNA, 21 bp, Artificial Sequence
  Pyrosquencing Primer
agggtttttatttttattaag

SEQ ID NO: 74 - DNA, 22 bp, Artificial Sequence
  Pyrosquencing Primer
gttaattaaattttttaatttg

SEQ ID NO: 75 - DNA, 25 bp, Artificial Sequence
  Pyrosquencing Primer
gatttttaaggaggggattttgtag

SEQ ID NO: 76 - DNA, 25 bp, Artificial Sequence
  Pyrosquencing Primer
ctatcccacatcaccaatctaaact

SEQ ID NO: 77 - DNA, 25 bp, Artificial Sequence
  Pyrosquencing Primer
tttttttattttatgtggtgtttgt

SEQ ID NO: 78 - DNA, 26 bp, Artificial Sequence
  Pyrosquencing Primer
actaaaactatccctacctattcctc

SEQ ID NO: 79 - DNA, 19 bp, Artificial Sequence
  Pyrosquencing Primer
ttttattttatgtggtgtt

SEQ ID NO: 80 - DNA, 19 bp, Artificial Sequence
  Pyrosquencing Primer
taattgtttttgttttttg

SEQ ID NO: 81 - DNA, 21 bp, Artificial Sequence
  Pyrosquencing Primer
gaattttagagtgaggatttg

SEQ ID NO: 82 - DNA, 27 bp, Artificial Sequence
  Pyrosquencing Primer
agaaggtttggggtaatatagaagttt

SEQ ID NO: 83 - DNA, 25 bp, Artificial Sequence
  Pyrosquencing Primer
aaatcccctctccttaaaacataac

SEQ ID NO: 84 - DNA, 20 bp, Artificial Sequence
  Pyrosquencing Primer
tgggaaattttttgatagtt

SEQ ID NO: 85 - DNA, 30 bp, Artificial Sequence
  Pyrosquencing Primer
aattctactaaatcctcaaaaaaactaaac

SEQ ID NO: 86 - DNA, 20 bp, Artificial Sequence
  Pyrosquencing Primer
tttgggttatatatgtgtta

SEQ ID NO: 87 - DNA, 19 bp, Artificial Sequence
  Pyrosquencing Primer
gtttttgtatttgtgaggg

SEQ ID NO: 88 - DNA, 25 bp, Artificial Sequence
  Pyrosquencing Primer
aagggaggtttttgttatttattta

SEQ ID NO: 89 - DNA, 24 bp, Artificial Sequence
  Pyrosquencing Primer
accttccttataaccatcaattcc

SEQ ID NO: 90 - DNA, 28 bp, Artificial Sequence
  Pyrosquencing Primer
tgttttttagtaagtgagaagagatggt

SEQ ID NO: 91 - DNA, 25 bp, Artificial Sequence
  Pyrosquencing Primer
aaccaaatttaaacccaacctaaac

SEQ ID NO: 92 - DNA, 24 bp, Artificial Sequence
  Pyrosquencing Primer
gtggtttagaaagaagagttttgt

SEQ ID NO: 93 - DNA, 25 bp, Artificial Sequence
  Pyrosquencing Primer
tgtggtaaggtttattttttagagg

SEQ ID NO: 94 - DNA, 26 bp, Artificial Sequence
  Pyrosquencing Primer
aaacaaacttttatctccccttttac

SEQ ID NO: 95 - DNA, 30 bp, Artificial Sequence
  Pyrosquencing Primer
ataatagaggatagaaataaaagaatgaaa

SEQ ID NO: 96 - DNA, 30 bp, Artificial Sequence
  Pyrosquencing Primer
ccttcaaattatataaatttcaaataataa

SEQ ID NO: 97 - DNA, 24 bp, Artificial Sequence
  Pyrosquencing Primer
attttagaagttataatttaagta

SEQ ID NO: 98 - DNA, 25 bp, Artificial Sequence
  Pyrosquencing Primer
ttttggaattagtttaaaagggttg

SEQ ID NO: 99 - DNA, 25 bp, Artificial Sequence
  Pyrosquencing Primer
aacaaaaaacaaaacaaaaccaaat

SEQ ID NO: 100 - DNA, 27 bp, Artificial Sequence
  Pyrosquencing Primer
atttttaagtagggatttttttgtgag

SEQ ID NO: 101 - DNA, 27 bp, Artificial Sequence
  Pyrosquencing Primer
tcatcctaataccttcaaactactctc

SEQ ID NO: 102 - DNA, 25 bp, Artificial Sequence
  Pyrosquencing Primer
gataagattttaatgtatttaagtt

SEQ ID NO: 103 - DNA, 25 bp, Artificial Sequence
  Pyrosquencing Primer
tatggattttgggatttttgattat

SEQ ID NO: 104 - DNA, 25 bp, Artificial Sequence
  Pyrosquencing Primer
tacaacaacctaaccaacaaaaaaa

SEQ ID NO: 105 - DNA, 30 bp, Artificial Sequence
  Pyrosquencing Primer
ttttaattttgaatttagaggaaatttagt

SEQ ID NO: 106 - DNA, 28 bp, Artificial Sequence
  Pyrosquencing Primer
aaaatatttaataaatttctattccccc

SEQ ID NO: 107 - DNA, 23 bp, Artificial Sequence
  Pyrosquencing Primer
gttatttgtatttttgttaaatt

SEQ ID NO: 108 - DNA, 21 bp, Artificial Sequence
  Pyrosquencing Primer
ttgtaaagtgtttgttgataa

SEQ ID NO: 109 - DNA, 23 bp, Artificial Sequence
  Pyrosquencing Primer
attagatatttttgttataattt

SEQ ID NO: 110 - DNA, 30 bp, Artificial Sequence
  Pyrosquencing Primer
tatgtgtatttatatttgggatttttttag

SEQ ID NO: 111 - DNA, 27 bp, Artificial Sequence
  Pyrosquencing Primer
aataaaactcttctaacccctaaactc

SEQ ID NO: 112 - DNA, 25 bp, Artificial Sequence
  Pyrosquencing Primer
gaagtttgtgttgtttgtatttttg

SEQ ID NO: 113 - DNA, 26 bp, Artificial Sequence
  Pyrosquencing Primer
tctatatttacttaaaacataccctc

SEQ ID NO: 114 - DNA, 26 bp, Artificial Sequence
  Pyrosquencing Primer
ggttttggtgtttttggagtgggaga

SEQ ID NO: 115 - DNA, 24 bp, Artificial Sequence
  Pyrosquencing Primer
gttatgtagtaggtgaaatgagtt

SEQ ID NO: 116 - DNA, 30 bp, Artificial Sequence
  Pyrosquencing Primer
ttgttgtataggtttgtagtttaggagtaa

SEQ ID NO: 117 - DNA, 26 bp, Artificial Sequence
  Pyrosquencing Primer
tcaaaaacccaattaaatcaatattc

SEQ ID NO: 118 - DNA, 30 bp, Artificial Sequence
  Pyrosquencing Primer
aaaattgtttaagagtatattttttaaaaa

SEQ ID NO: 119 - DNA, 25 bp, Artificial Sequence
  Pyrosquencing Primer
aacatccaacattaatttccttacc

SEQ ID NO: 120 - DNA, 27 bp, Artificial Sequence
  Pyrosquencing Primer
atttttattgttaagttatatatgatt

SEQ ID NO: 121 - DNA, 22 bp, Artificial Sequence
  Pyrosquencing Primer
tagaggttttttaaagaatgtg

SEQ ID NO: 122 - DNA, 23 bp, Artificial Sequence
  Pyrosquencing Primer
attttgtaaattaaattagatgg

SEQ ID NO: 123 - DNA, 30 bp, Artificial Sequence
  Pyrosquencing Primer
tatagaatgatgtttataaagtgggatata

SEQ ID NO: 124 - DNA, 26 bp, Artificial Sequence
  Pyrosquencing Primer
aattaaaaaccccataacactcttct

SEQ ID NO: 125 - DNA, 30 bp, Artificial Sequence
  Pyrosquencing Primer
gggatataaatatatatatttatgtaattt

SEQ ID NO: 126 - DNA, 25 bp, Artificial Sequence
  Pyrosquencing Primer
aaccaatatttccttcaaaaaacaa

SEQ ID NO: 127 - DNA, 23 bp, Artificial Sequence
  Pyrosquencing Primer
agaagtgtgttgtttagagtggt

SEQ ID NO: 128 - DNA, 19 bp, Artificial Sequence
  Quantitative PCR Primer
cccaggaccctcactccat

SEQ ID NO: 129 - DNA, 21 bp, Artificial Sequence
  Quantitative PCR Primer
tcccaaactcccagtcttgaa

SEQ ID NO: 130 - DNA, 19 bp, Artificial Sequence
  Quantitative PCR Primer
gccaggttgcctttggagt

SEQ ID NO: 131 - DNA, 21 bp, Artificial Sequence
  Quantitative PCR Primer
tcccggatgttgacacaggta

SEQ ID NO: 132 - DNA, 22 bp, Artificial Sequence
  Quantitative PCR Primer
ccccgtctgaggtaatagttgt

SEQ ID NO: 133 - DNA, 23 bp, Artificial Sequence
  Quantitative PCR Primer
gagcagtctccaggagtatagtc

SEQ ID NO: 134 - DNA, 23 bp, Artificial Sequence
  Quantitative PCR Primer
gaagctaaagtaaaagcccacga

SEQ ID NO: 135 - DNA, 23 bp, Artificial Sequence
  Quantitative PCR Primer
acacatcagaggtgttgacaatg

SEQ ID NO: 136 - DNA, 22 bp, Artificial Sequence
  Quantitative PCR Primer
aacgaacacagcgaatgtttcc

SEQ ID NO: 137 - DNA, 21 bp, Artificial Sequence
  Quantitative PCR Primer
caccttgtatgtagcgaacgc

SEQ ID NO: 138 - DNA, 23 bp, Artificial Sequence
  Quantitative PCR Primer
ctgaagttccaaacagttcgagg

SEQ ID NO: 139 - DNA, 19 bp, Artificial Sequence
  Quantitative PCR Primer
cgcacgaaacactcggatg

SEQ ID NO: 140 - DNA, 21 bp, Artificial Sequence
  Quantitative PCR Primer
gcacaagtcgcagctacttct

SEQ ID NO: 141 - DNA, 21 bp, Artificial Sequence
  Quantitative PCR Primer
ggagtggagtcctttttctgg

SEQ ID NO: 142 - DNA, 19 bp, Artificial Sequence
  Quantitative PCR Primer
ggtggcacgggaatgagtc

SEQ ID NO: 143 - DNA, 22 bp, Artificial Sequence
  Quantitative PCR Primer
gatttgtactggcgtctccttt

SEQ ID NO: 144 - DNA, 21 bp, Artificial Sequence
  Quantitative PCR Primer
ctgcataacggtctggacttc

SEQ ID NO: 145 - DNA, 19 bp, Artificial Sequence
  Quantitative PCR Primer
cagcaactgcccgtactcc

SEQ ID NO: 146 - DNA, 21 bp, Artificial Sequence
  Quantitative PCR Primer
gcttcctgtaggacaagacct

SEQ ID NO: 147 - DNA, 19 bp, Artificial Sequence
  Quantitative PCR Primer
ggctttgagggggcttagg

SEQ ID NO: 148 - DNA, 19 bp, Artificial Sequence
  Quantitative PCR Primer
ggaccctcactctcggatg

SEQ ID NO: 149 - DNA, 20 bp, Artificial Sequence
  Quantitative PCR Primer
ttctggcaggggttcactct

SEQ ID NO: 150 - DNA, 23 bp, Artificial Sequence
  Quantitative PCR Primer
gggaatgtactgaagacatctgc

SEQ ID NO: 151 - DNA, 23 bp, Artificial Sequence
  Quantitative PCR Primer
ccacagccataatacctcgaact

SEQ ID NO: 152 - DNA, 22 bp, Artificial Sequence
  Quantitative PCR Primer
acgtggtgaatacatcaagacc

SEQ ID NO: 153 - DNA, 23 bp, Artificial Sequence
  Quantitative PCR Primer
gctacagagaaattgttcagggg

SEQ ID NO: 154 - DNA, 21 bp, Artificial Sequence
  Quantitative PCR Primer
tcagctcaaagagtctggcac

SEQ ID NO: 155 - DNA, 22 bp, Artificial Sequence
  Quantitative PCR Primer
ggtaaagctcgtctagcacctc